(19) United States
(12) Patent Application Publication
(10) Pub. No.: US 2024/0301074 A1
(43) Pub. Date: Sep. 12, 2024

(54) USE OF ANTI-IL-36R ANTIBODIES FOR THE TREATMENT OF HIDRADENTITIS SUPPURATIVA (HS)

(71) Applicant: Boehringer Ingelheim International GmbH, Ingelheim am Rhein (DE)

(72) Inventors: Ahmed Karim FARAG, Biberach an der Riss (DE); Nathalie IVANOFF, Mittelbiberach (DE); Sree KURUP, Long Island City, NY (US); Xiujiang LI, Ridgefield, CT (US); Sutirtha MUKHOPADHYAY, Ingelheim am Rhein (DE); Usha RANGANATHAN, Lawrence Township, NJ (US)

(21) Appl. No.: 18/599,267

(22) Filed: Mar. 8, 2024

Related U.S. Application Data
(60) Provisional application No. 63/621,291, filed on Jan. 16, 2024.
(60) Provisional application No. 63/489,246, filed on Mar. 9, 2023.

(51) Int. Cl. C07K 16/28; A61K 39/00; A61P 17/00

(52) U.S. Cl. CPC C07K 16/2866; A61P 17/00; A61K 2039/505; A61K 2039/54; A61K 2039/545

(57) ABSTRACT

The invention provides methods, uses and compositions for the treatment of hidradenitis suppurativa. The invention describes methods and uses for treating hidradenitis suppurativa with anti-interleukin 36R (anti-IL36R) antibodies. Also described are methods for determining the efficacy of anti-IL36R antibodies for treating hidradenitis suppurativa in a subject.

### SEQUENCE LISTING

The instant application contains a Sequence Listing which has been submitted in XML format via Patent Center and is hereby incorporated by reference in its entirety. Said XML copy, created on Mar. 27, 2024, is named 09-0743-US-3_Sequence_Listing-Rectification_SL.xml and is 137,371 bytes in size.

### TECHNICAL FIELD OF THE INVENTION

The present invention relates to the administration of an anti-interleukin-36 receptor (anti-IL-36R) antibody to a subject with a hidradenitis suppurativa (HS) and the treatment and/or prevention of the hidradenitis suppurativa (HS) in the subject. More specifically, the invention relates to the administration of spesolimab to a subject suffering from hidradenitis suppurativa (HS).

### BACKGROUND

Hidradenitis suppurativa (HS), also called acne inversa is a rare, chronic inflammatory skin disease characterized by recurrent, painful abscesses and fistulous tracts (also called draining tunnels). Patients with HS objectively have one of the lowest quality of life measures of any dermatologic disease. Lesions characteristically occur in the axillary, groin, infra mammary, or anogenital regions of the body. HS lesions may progress to form sinus tracts and expansive abscesses. Sequelae include significant pain, scarring, and psychological distress. The average age of onset is during the early 20s (Zouboulis C C, Marmol V del, Mrowietz U, et al. Hidradenitis suppurativa/acne inversa: criteria for diagnosis, severity assessment, classification and disease evaluation. Dermatology (Basel) 2015; 231(2):184-190).

The global prevalence of HS is reported to be 0.3% globally, ranging from 0.2% to 0.6%, with variability across geographic settings. (Phan K, Charlton O, Smith S D. Global prevalence of hidradenitis suppurativa and geographical variation-systematic review and metaanalysis. Biomedical Dermatology. 2020; 4(2):1-6). Underdiagnosis or improper diagnosis is common. Overall, HS prevalence varies significantly based on study methodology; however, the disease appears to be more common than was previously considered (Saunte D M, Boer J, Stratigos A, et al. Diagnostic delay in hidradenitis suppurativa is a global problem. Br J Dermatol 2015; 173(6):1546-1549).

Some of the most burdensome HS symptoms from patient perspective are pain, drainage and explosive openings, itch, skin tightness (scarring), odour, fatigue, and flu-like symptoms. Patients reported to be unsatisfied with the level of control offered by currently available treatment options; unmet needs from the patient perspective include the need for new medical treatments with favorable efficacy and tolerability profiles. In qualitative evidence, the most important treatment goals from the patient perspective were pain, drainage (including explosive openings), and fatigue.

Auto-inflammation is suggested to play a role in the pathogenesis. The exact cytokine profile of HS has yet to be determined, although abnormal levels of several inflammatory cytokines have been observed. Protein quantities of TNFα were also elevated in these patients.

A genetic basis for HS has been identified in some families with history of HS The earliest mutation identified in families with HS was in γ-secretase, an endoprotease complex that cleaves numerous Type-1 transmembrane proteins and is encoded by several genes, including NCSTN, PSENEN and PSEN1. Loss-of-function mutations within the NCSTN gene (which encodes one of the four subunits of γ-secretase68) support a key role for the γ-secretase complex in HS. Although robust correlations between genotype and phenotype are lacking, the observed clinical phenotype is severe and extensive in NCSTN mutation-positive patients with HS. In most patients, HS is triggered by environmental factors in genetically predisposed people (Scala E et al. Hidradenitis Suppurativa: Where We Are and Where We Are Going. Cells 2021; 10; van Straalen K R, et al. Contribution of Genetics to the Susceptibility to Hidradenitis Suppurativa in a Large, Cross-sectional Dutch Twin Cohort. JAMA Dermatol 2020; 156:1359-62; Pink A E, et al. Mutations in the γ-secretase genes NCSTN, PSENEN, and PSEN1 underlie rare forms of hidradenitis suppurativa (acne inversa). Journal of Investigative Dermatology 2012; 132:2459-61; Moltrasio C, et al. Hidradenitis Suppurativa: A Perspective on Genetic Factors Involved in the Disease. Biomedicines 2022; 10; Gao M, Wang P G, Cui Y, et al. Inversa acne (hidradenitis suppurativa): a case report and identification of the locus at chromosome 1p21.1-1925.3. J Invest Dermatol 2006; 126:1302-6; and Nomura Y, Nomura T, Suzuki S, et al. A novel NCSTN mutation alone may be insufficient for the development of familial hidradenitis suppurativa. J Dermatol Sci 2014; 74:180-2).

Treatment of HS often begins with topical or oral antibiotics, such as topical clindamycin and oral tetracycline, followed by the use of rifampicin if there is no improvement. When topical medications and oral antibiotics fail, or the disease has progressed, biologics are recommended (Zouboulis C C, Desai N, Emtestam L, et al. European S1 guideline for the treatment of hidradenitis suppurativa/acne inversa. J Eur Acad Dermatol Venereol 2015; 29:619-644). While the TNF-α antagonist, Adalimumab is the only approved biologic, the response rate is 42%-59% versus placebo response of 26%-28%, with a schedule of q1w s.c. dosing. Biologics are an emerging therapeutic modality used in the management of many inflammatory conditions including HS. Implementation of biologics is typically reserved for moderate-to-severe cases or in those cases that are refractory to treatment. Other biologics including, for example, Infliximab (anti-TNF-α, one open-label phase II study with 17 patients), Golimumab (anti-TNF-α, no clinical studies just case reports), Enteracept (soluble TNF receptor, double-blinded study), Ustekinumab (anti-IL-12/IL-23, one open-label phase II study with 17 patients), Guselkumab (anti-IL-23, Ph II), Risankizumab (anti-IL-23, PhII), Anakinra (anti-IL1α, one open-label phase II study with 17 patients), Bermekimab (anti-IL1α, one double-blind randomized clinical trial (RCT) of 20 patients, one open-label clinical trial with 6 patient), secukinumab (anti-IL-17, clinical trials ongoing), bimekizumab (anti-IL-17A/F, PhIII met the primary endpoint, demonstrating statistically significant and consistent clinically meaningful improvements), brodalumab (anti-IL-17R, Ph II ongoing) are being tested with limited results, although recent results with the drugs targeting the IL-17 pathway, bimekizumab and brodalumab, have shown real promise (Flood K S, Porter M L, Kimball A B. Biologic Treatment for Hidradenitis Suppurativa. Am J Clin Dermatol. 2019 October; 20(5):625-638; Glatt S, Jemec G B E, Forman S, et al. Efficacy and Safety of Bimekizumab in Moderate to Severe Hidradenitis Suppurativa: A Phase 2, Double-blind, Placebo-Controlled Randomized Clinical Trial. JAMA Dermatol. 2021; 157(11):1279-1288; Frew J W, Navrazhina K, Grand D, Sullivan-Whalen M, Gilleaudeau P, Garcet S, Ungar J, Krueger J G. The effect of subcutaneous brodalumab on clinical disease activity in hidradenitis suppurativa: An open-label cohort study. J Am Acad Dermatol. 2020 November; 83(5): 1341-1348). When the medical management is ineffective, surgery is the only option.

There are also studies into the relationship between IL-36 and the pathogenesis of HS. However, in a recent clinical trial of an anti-IL36 R antagonist (imsidolimab) for the treatment of moderate-to-severe hidradenitis suppurativa (HS), there was no demonstrable improvement over placebo in the primary endpoint and key secondary endpoints for HS (See A Study to Evaluate the Efficacy and Safety of Imsidolimab (ANB019) in the Treatment of Subjects With Hidradenitis Suppurativa ClinicalTrials.gov NCT04856930; Anaptysbio Reports HARP Phase 2 Top-line Data of Imsidolimab in Moderate to Severe Hidradenitis Suppurativa, Aug. 31, 2022).

Thus, a need exists in the art for novel targeted therapies for the treatment and/or prevention of hidradenitis suppurativa (HS).

### SUMMARY OF THE INVENTION

Given the limited success of treatments for hidradenitis suppurativa (HS), there remains a need for an effective treatment, especially in view of the debilitating nature of this disease. The present invention addresses the above need by providing bio-therapeutics, in particular antibodies, which bind to IL-36R as a first-second-, third- or subsequent-line therapy for treating hidradenitis suppurativa.

In a first aspect, the present invention relates to a method for treating, preventing or ameliorating a hidradenitis suppurativa (HS) in a subject, comprising administering of having administered to the subject a therapeutically effective amount of an anti-IL-36R antibody or an antigen-binding fragment thereof. In an embodiment relating to this aspect, the anti-IL-36R antibody is spesolimab.

In second aspect, the present invention relates to treating moderate to severe HS in a patient, comprising administering or having administered to the patient a therapeutically effective amount of an anti-IL-36R antibody. In an embodiment relating to this aspect, the anti-IL-36R antibody is spesolimab.

In third aspect, the present invention relates to a method of treating a skin disorder associated with hidradenitis suppurativa (HS) in a patient, said method(s) including administering or having administered to the patient a therapeutically effective amount of an anti-IL-36R antibody of the present invention or an antigen binding fragment thereof. In an embodiment relating to this aspect, the anti-IL-36R antibody is spesolimab.

In a fourth aspect, the present invention relates to a method of reducing or alleviating signs and symptoms of HS in a patient, said method comprising administering or having administered to the patient a therapeutically effective amount of an anti-IL-36R antibody. In an embodiment relating to this aspect, the invention is a method of reducing or alleviating the number, prevalence of, severity of, and/or reoccurrence of draining tunnels of an HS patient, said method comprising administering or having administered to the patient a therapeutically effective amount of an anti-IL-36R antibody. In an embodiment relating to this aspect, the anti-IL-36R antibody is spesolimab.

In fifth aspect, the present invention relates to a method of treating skin inflammation associated with hidradenitis suppurativa (HS) in a subject, said method including administering or having administered to the subject a therapeutically effective amount of an anti-IL-36R antibody of the present invention or an antigen binding fragment thereof. In an embodiment relating to this aspect, the anti-IL-36R antibody is spesolimab. In a related embodiment, the present invention relates to a method preventing or reducing neutrophilic infiltrates in affected skin tissues in a subject suffering from hidradenitis suppurativa (HS), said method including administering or having administered to the subject a therapeutically effective amount of an anti-IL-36R antibody of the present invention or an antigen binding fragment thereof. In an embodiment relating to this aspect, the anti-IL-36R antibody is spesolimab. In an embodiment related to the present invention relates to a method of reducing susceptibility to a skin infection in a subject with hidradenitis suppurativa (HS) comprising administering to the subject a therapeutically effective amount of an anti-IL-36-R antibody or an antigen-binding fragment thereof (as disclosed herein). In an embodiment relating to this aspect, the anti-IL-36R antibody is spesolimab.

In a sixth aspect, the present invention relates to a method of reducing the severity and duration an acute flare of hidradenitis suppurativa (HS), said method comprising including administering or having administered to the patient a therapeutically effective amount of an anti-IL-36R antibody of the present invention or an antigen binding fragment thereof. In an embodiment relating to this aspect, the anti-IL-36R antibody is spesolimab.

In an embodiment relating to any of the aspects first to sixth, a second therapeutic agent is administered to the subject before, after, or concurrent with the anti-IL-36R antibody or an antigen-binding fragment thereof. In a related embodiment, the second therapeutic agent is selected from the group consisting of an anti-bacterial agent, an anti-viral agent, an anti-fungal agent, another IL-36R antagonist, an anti PDE4, an IL-17 antagonist, an IL-12/IL-23 antagonist, an IL-23 antagonist, and IL-1 antagonist, an IgE inhibitor, a corticosteroid, NSAID, an IL-4R antagonist, a TNF-α inhibitor, and IFNγ.

In an embodiment relating to any of aspects first to sixth, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of aspects first to sixth, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of aspects first to sixth, the anti-IL-36R antibody includes:
i. a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
ii. a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iii. a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iv. a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
v. a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
vi. a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
vii. a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of aspects first to sixth, the anti-IL-36R antibody includes:
i. a light chain variable region comprising the amino acid sequence of SEQ ID NO: 77; and a heavy chain variable region comprising the amino acid sequence of SEQ ID NO: 87; or
ii. a light chain variable region comprising the amino acid sequence of SEQ ID NO: 77; and a heavy chain variable region comprising the amino acid sequence of SEQ ID NO: 88; or
iii. a light chain variable region comprising the amino acid sequence of SEQ ID NO: 77; and a heavy chain variable region comprising the amino acid sequence of SEQ ID NO: 89; or
iv. a light chain variable region comprising the amino acid sequence of SEQ ID NO: 80; and a heavy chain variable region comprising the amino acid sequence of SEQ ID NO: 87; or
v. a light chain variable region comprising the amino acid sequence of SEQ ID NO: 80; and a heavy chain variable region comprising the amino acid sequence of SEQ ID NO: 88; or
vi. a light chain variable region comprising the amino acid sequence of SEQ ID NO: 80; and a heavy chain variable region comprising the amino acid sequence of SEQ ID NO: 89; or
vii. a light chain variable region comprising the amino acid sequence of SEQ ID NO: 85; and a heavy chain variable region comprising the amino acid sequence of SEQ ID NO: 100; or
viii. a light chain variable region comprising the amino acid sequence of SEQ ID NO: 85; and a heavy chain variable region comprising the amino acid sequence of SEQ ID NO:101; or
ix. a light chain variable region comprising the amino acid sequence of SEQ ID NO: 86; and a heavy chain variable region comprising the amino acid sequence of SEQ ID NO: 100; or
x. a light chain variable region comprising the amino acid sequence of SEQ ID NO: 86; and a heavy chain variable region comprising the amino acid sequence of SEQ ID NO:101.

In an embodiment relating to any of aspects first to sixth, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In one embodiment, relating to any of aspects first to sixth, the invention provides a method for treating a subject having hidradenitis suppurativa (HS), the method comprising administering an isolated human anti IL-36R antibody, or an antigen binding portion thereof, to the subject according to a multiple variable dose regimen, such that HS is treated, wherein the multiple variable dose regimen comprises administering at least one or more parenteral (e.g. intravenous or subcutaneous) doses. In a related embodiment, each of the one or more parenteral dose(s) includes at least 450 mg, 600 mg, 900 mg, 1200 mg, or 1800 mg, 2000 mg, or 3000 mg of said anti-IL-36R antibody, wherein the total loading dose is at least 900 mg, 1200 mg, 1350 mg, 1600 mg, 1800 mg, 2000 mg, 2400 mg, 2700 mg, 3000 mg, 3600 mg, 4800 mg, 6000 mg, 7200 mg, or 8000 mg. In a related embodiment, the anti-IL-36R antibody is administered in a loading dose of at least 1800 mg comprising one or more parenteral doses of 450 mg, 600 mg, 900 mg, 1200 mg, or 1800 mg of said anti-IL-36R antibody. In a related embodiment, the anti-IL-36R antibody is administered in one, two, three, or four parenteral doses (qw, q2w, q4w) of 450 mg; one, two, three, or four parenteral doses of 600 mg (qw, q2w, q4w); one, two, three, or four parenteral doses (qw, q2w, q4w) of 900 mg; one, two, three, or four parenteral doses (qw, q2w, q4w) of 1200 mg; one, two, three, or four parenteral doses (qw, q2w, q4w) of 1800 mg, one, two, three, or four parenteral doses (qw, q2w, q4w) of 2000 mg; one or two parenteral doses (q2w, q4w) of 2400 mg; one or two parenteral doses (q2w, q4w) of 3000 mg, or any combination thereof to achieve an efficacious dose. In a related embodiment 1 2, 3, or 4, parenteral doses are administered at 1 (qw), 2 (q2w), or 4-week (q4w) intervals.

In one embodiment, relating to any of aspects first to sixth, the invention provides a method for treating a subject having hidradenitis suppurativa (HS), the method comprising administering an isolated human anti IL-36R antibody, or an antigen binding portion thereof, to the subject according to a multiple variable dose regimen, such that HS is treated, wherein the multiple variable dose regimen comprises administering at least one or more intravenous doses. In a related embodiment, each of the one or more intravenous dose(s) includes at least 450 mg, 600 mg, 900 mg, 1200 mg, or 1800 mg, 2000 mg, or 3000 mg of said anti-IL-36R antibody, wherein the total loading dose is at least 900 mg, 1200 mg, 1350 mg, 1600 mg, 1800 mg, 2000 mg, 2400 mg, 2700 mg, 3000 mg, 3600 mg, 4800 mg, 5400 mg, 6000 mg, 7200 mg, or 8000 mg. In a related embodiment, the anti-IL-36R antibody is administered in a loading dose of at least 1800 mg comprising one or more intravenous doses of 450 mg, 600 mg, 900 mg, 1200 mg, or 1800 mg of said anti-IL-36R antibody. In a related embodiment, the anti-IL-36R antibody is administered in one, two, three, or four intravenous doses (qw, q2w, q4w) of 450 mg; one, two, three, or four parenteral doses of 600 mg (qw, q2w, q4w); one, two, three, or four intravenous doses (qw, q2w, q4w) of 900 mg; one, two, three, or four Intravenous doses (qw, q2w, q4w) of 1200 mg; one, two, three, or four intravenous doses (qw, q2w, q4w) of 1800 mg, one, two, three, or four intravenous doses (qw, q2w, q4w) of 2000 mg; one or two intravenous doses (q2w, q4w) of 2400 mg; one or two intravenous doses (q2w, q4w) of 3000 mg; or any combination thereof to achieve an efficacious dose.

In one embodiment, relating to any of aspects first to sixth, the invention provides a method for treating a subject having hidradenitis suppurativa (HS), the method comprising administering an isolated human anti IL-36R antibody, or an antigen binding portion thereof, to the subject according to a multiple variable dose regimen, such that HS is treated, wherein the multiple variable dose regimen comprises administering at least one or more subcutaneous doses. In a related embodiment, each of the one or more subcutaneous dose(s) includes at least 450 mg, 600 mg, 900 mg, 1200 mg, or 1800 mg, 2000 mg, or 3000 mg of said anti-IL-36R antibody, wherein the total loading dose is at least 900 mg, 1200 mg, 1350 mg, 1600 mg, 1800 mg, 2000 mg, 2400 mg, 2700 mg, 3000 mg, 3600 mg, 4800 mg, 5400 mg, 6000 mg, 7200 mg, or 8000 mg. In a related embodiment, the anti-IL-36R antibody is administered in a loading dose of at least 1800 mg comprising one or more subcutaneous doses of 450 mg, 600 mg, 900 mg, 1200 mg, 1800 mg, 2400 mg, or 2000 mg of said anti-IL-36R antibody. In a related embodiment, the anti-IL-36R antibody is administered in one, two, three, or four subcutaneous doses (qw, q2w, q4w) of 450 mg; one, two, three, or four subcutaneous doses of 600 mg (qw, q2w, q4w); one, two, three, or four subcutaneous doses (qw, q2w, q4w) of 900 mg; one, two, three, or four subcutaneous doses (qw, q2w, q4w) of 1200 mg; one, two, three, or four subcutaneous doses (qw, q2w, q4w) of 1800 mg; one, two, three, or four subcutaneous doses (qw, q2w, q4w) of 2000 mg; one or two subcutaneous doses (qw, q2w, q4w) of 2400 mg; one or two subcutaneous doses (qw, q2w, q4w) of 3000 mg; or any combination thereof to achieve an efficacious dose. In a related embodiment 1,2, 3, or 4, subcutaneous doses are administered at 1 (qw), 2 (q2w), or 4-week (q4w) intervals.

In one embodiment related to any of the above aspects and embodiments, the anti-IL-36R antibody is administered in one or more intravenous dose(s) or subcutaneous dose(s), wherein the total loading dose is at least 1200 mg, 1800 mg, 3000 mg, 3600 mg, 6000 mg, 7200 mg, or 8000 mg. In non-limiting, exemplary embodiments four intravenous doses of 450 mg are delivered at weeks 0, 1, 2, and 3 for a total loading dose of 1800 mg; four intravenous doses of 900 mg are delivered at weeks 0, 1, and 2, 3 for a total loading dose of 3600 mg; or four intravenous doses of 1800 mg are delivered at weeks 0, 1, and 2, 3 for a total loading dose of 7200 mg.

TABLE 1
Exemplary Loading Dosing:

| | LOADING DOSE (0-4 WEEKS) | | | | | | | |
|---|---|---|---|---|---|---|---|---|
| TOTAL | 1800 | 2400 | 3600 | 4800 | 7200 | 8000 | 4800 | 6000 |
| LOADING DOSE | MG | MG | MG | MG | MG | MG | MG | MG |
| EACH DOSE | 450 | 600 | 900 | 1200 | 1800 | 2000 | 2400 | 3000 |
| | MG | MG | MG | MG | MG | MG | MG | MG |
| RANGE | 450- | 600- | 900- | 1200- | 1800- | 2000- | 2400- | 3000- |
| | 1800 | 2400 | 3600 | 4800 | 7200 | 8000 | 4800 | 6000 |
| FREQUENCY | QW, | QW, | QW, | QW, | QW, | QW, | Q2W/ | Q2W/ |
| | Q2W, Q4W | Q2W, Q4W | Q2W, Q4W | Q2W, Q4W | Q2W, Q4W | Q2W, Q4W | Q4W | Q4W |
| TIMING (D 0, | 0, 1, | 0, 1, | 0, 1, | 0, 1, | 0, 1, | 0, 1, | | |
| W 1, W 2, W 3) | 2, 3 | 2, 3 | 2, 3 | 2, 3 | 2, 3 | 2, 3 | | |
| (D 0, W 2) | 0, 3 | 0, 3 | 0, 3 | 0, 3 | 0, 3 | 0, 3 | 0, 3 | 0, 3 |
| (D 0) | 0 | 0 | 0 | 0 | 0 | 0 | 0 | 0 |
| ADMINISTRATION | IV/SC | IV/SC | IV/SC | IV/SC | IV/SC | IV/SC | IV/SC | IV/SC |

In another embodiment related to any of aspects first to sixth, 1, 2, 3, or 4 intravenous dose(s) is/are administered as a loading dose, followed by at least one, 2, 3, or, 4 subcutaneous dose(s) is/are administered as maintenance dose(s) to the subject at 4, 5, 6, 7-, 8-, 10-12-, or 16-weeks at 1, 2, or 4 week intervals (qw, q2w, q4w) after the last intravenous dose, for example after the completion of the loading dose.

In another embodiment related to any of aspects first to sixth, 1, 2, 3, or 4 subcutaneous dose(s) is/are administered as a loading dose, followed by at least one, 2, 3, or 4 subcutaneous dose(s) is/are administered to the subject as maintenance dose(s) at 4, 5, 6, 7-, 8-, 10- or 12, -16-weeks at 1, 2, or 4 week intervals (qw, q2w, q4w) intervals after the last subcutaneous dose, for example after the completion of the loading dose.

In another embodiment, related to any of aspects first to sixth, the invention comprises administering or having administered to the subject an isolated human anti IL-36R antibody, or an antigen binding portion thereof, according to a multiple variable dose regimen, wherein the multiple variable dose regimen comprises administering a loading dose comprising at least 1, 2 3, 4 intravenous dose(s) or subcutaneous dose(s) of 450 mg, 600 mg, 900 mg, 1200 mg, 1800 mg, 2000 mg, 2400 mg, 3000 mg at 1, 2, or 4 week intervals (qw, q2w, q4w) for the first 4 weeks, and administering a maintenance dose comprising at least 1, 2, 3, 4, or 5 subcutaneous dose(s), wherein the first subcutaneous dose of the maintenance dose is administered to the subject after the last intravenous dose of the loading dose at 1, 2, 3, 4, 5, 6, 7, 8, 10, 12, or 16-weeks at 1, 2, or 4 week intervals (qw, q2w, q4w) from week 4.

In one embodiment related to any of the above aspects and embodiments, the anti-IL-36R antibody is administered as a maintenance dose in one or more intravenous or subcutaneous dose(s) from week 4, wherein the total maintenance dose is at least 1200 mg, 1800 mg, 2400 mg, 3000 mg, 3600 mg, 4800 mg, or 6000 mg. In a preferred embodiment, one, two, three, or four subcutaneous doses of 300 mg, 450 mg, 600 mg, 900 mg, 1200 mg, 1800 mg, 2400 mg, 3000 mg, or any combination thereof are delivered at weeks 4, 5, 6, 7, 8, 9, 10, 11, 12, 13, 14, 15, or 16 every 1, 2, or 4 weeks (qw, q2w, q4w).

In one embodiment related to any of the above aspects and embodiments, the anti-IL-36R antibody is administered in one or more intravenous or subcutaneous dose(s), wherein the total loading dose is at least 1200 mg, 1800 mg, 2400 mg, 3000 mg, 3600 mg, 4800 mg, 6000 mg, 7200 mg, or 8000 mg for the first 4 weeks followed by one, two, three, four, five or six subcutaneous maintenance doses of 300 mg, 450 mg, 600 mg, 900 mg, 1200 mg, 1800 mg, 2400 mg, or 3000 mg or combinations thereof, delivered at weeks 4, 5, 6, 7, 8, 9, 10, 11, 12, 13, 14, 15, or 16 every 1, 2, or 4 weeks (qw, q2w, q4w). In a preferred embodiment, a maintenance dose is delivered as four subcutaneous doses of 300, 450, 600, 900 mg, 1200 mg, are delivered weekly (qw) between week 4 and week 8 for a total maintenance dose of 1200 mg, 1800 mg, 2400 mg, 3600 mg, or 4800 mg. In an alternative embodiment, a maintenance dose is delivered as two subcutaneous doses of 2400 mg or 3000 mg, delivered every two weeks between week 4 and week 8 for a total maintenance dose of 4800 mg or 6000 mg. In an alternative embodiment, a maintenance dose is delivered once for a total subcutaneous dose of 4800 mg or 6000 mg, delivered between week 4 and week 8 for a total maintenance dose of 4800 mg or 6000 mg.

In one embodiment related to any of the above aspects and embodiments, the anti-IL-36R antibody is administered in one or more intravenous dose(s) or subcutaneous dose(s), wherein the total loading dose is 1800 mg (e.g., 450 mg/qw), followed by at least one, two, three, four, five, or six subcutaneous doses of 300 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous dose(s), wherein the total loading dose is 3600 mg (e.g., 900 mg/qw), followed by at least one, two, three, four, five, or six subcutaneous doses of 600 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous dose(s), wherein the total loading dose is 3600 mg (e.g., 900 mg/qw), followed by at least one, two, three, four, five, or six subcutaneous doses of 1200 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous dose(s), wherein the total loading dose is 7200 mg (e.g., 1800 mg/qw), followed by at least one, two, three, four, five, or six subcutaneous doses of 1200 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous, wherein the total loading dose is 6000 mg (e.g., 3000 mg/q2w), followed by at least one, two, three, four, five, or six subcutaneous doses of 600 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous dose(s), wherein the total loading dose is 6000 mg, followed by one, two, three, four, five, or six subcutaneous doses of 1200 mg every two weeks, for example at weeks 4, 6, 8, 10, 12, or 14.

In one embodiment related to any of the above aspects and embodiments, the anti-IL-36R antibody is administered in one or more intravenous dose(s) or subcutaneous dose(s), wherein the total loading dose is at least 1200 mg, 1800 mg, 3000 mg, 3600 mg, 7200 mg, or 8000 mg, followed by at least one subcutaneous dose of 300 mg, 600 mg, or 1200 mg every week at weeks 4, 5, 6, and 7. In non-limiting, exemplary embodiments, four subcutaneous doses of 300 mg are delivered once weekly at weeks 4, 5, 6, and 7 for a total maintenance dose of 1200 mg; four subcutaneous doses of 600 mg are delivered once weekly at weeks 4, 5, 6, and 7 for a total maintenance dose of 2400 mg; or four subcutaneous doses of 1200 mg are delivered once weekly at weeks 4, 5, 6, and 7 for a total maintenance dose of 3600 mg. In a related embodiment, the at least one maintenance dose is administered after the final dose comprising the loading dose.

TABLE 2
Exemplary Maintenance Dosing Weeks 4-8:

| | Maintenance DOSE (4-8 WEEKS) | | | | | | | |
|---|---|---|---|---|---|---|---|---|
| TOTAL | 1200 | 1800 | 2400 | 3600 | 4800 | 3600 | 4800 | 6000 |
| LOADING DOSE | MG | MG | MG | MG | MG | MG | MG | MG |
| EACH DOSE | 300 | 450 | 600 | 900 | 1200 | 1800 | 2400 | 3000 |
| | MG | MG | MG | MG | MG | MG | MG | MG |
| RANGE | 300- | 450- | 600- | 900- | 1200- | 1800- | 2400- | 3000- |
| | 1200 | 1800 | 2400 | 3600 | 4800 | 3600 | 4800 | 6000 |
| FREQUENCY | QW, | QW, | QW, | QW, | QW, | Q2W/ | Q2W/ | Q2W/ |
| | Q2W, Q4W | Q2W, Q4W | Q2W, Q4W | Q2W, Q4W | Q2W, Q4W | Q4W | Q4W | Q4W |
| TIMING (W 4- | 4, 5, | 4, 5, | 4, 5, | 4, 5, | 4, 5, | | | |
| W 8/QW) | 6, 7 | 6, 7 | 6, 7 | 6, 7 | 6, 7 | | | |
| (W 4-W 8, Q2W) | 4, 6 | 4, 6 | 4, 6 | 4, 6 | 4, 6 | 4, 6 | 4, 6 | 4, 6 |
| (W 4, Q4W) | 4 | 4 | 4 | 4 | 4 | 4 | 4 | 4 |
| ADMINISTRATION | SC | SC | SC | SC | SC | SC | SC | SC |

In one embodiment related to any of the above aspects and embodiments, the anti-IL-36R antibody maintenance therapy can be continued after week 8 comprising at least one subcutaneous dose(s), wherein subcutaneous dosing remains the same, or can be titrated up or down, and wherein each dose is 600 mg, 900 mg, 1200 mg, 1800 mg, 2400 mg, or 3000 mg, administered every 2 (q2w) or 4 (q4w) weeks after week 8 following the last subcutaneous maintenance dose (e.g. at between 4-8 weeks). In non-limiting, exemplary embodiments, four subcutaneous doses of 300 mg are delivered once weekly at weeks 4, 5, 6, and 7 for a total maintenance dose of 1200 mg followed by at least one 600 mg, 900 mg, 1200 mg, 1800 mg, 2400 mg, or 3000 mg subcutaneous dose that is administered between weeks 8 and 52, preferably every two weeks (q2w). In an alternative embodiment four subcutaneous doses of 600 mg are delivered once weekly at weeks 4, 5, 6, and 7 for a total maintenance dose of 2400 mg; followed by at least one 600 mg, 900 mg, 1200 mg, 1800 mg, 2400 mg, or 3000 mg subcutaneous dose that is administered between weeks 8 and 52, preferably every two weeks (q2w). In yet another alternative embodiment, four subcutaneous doses of 1200 mg are delivered once weekly at weeks 4, 5, 6, and 7 for a total maintenance dose of 3600 mg, followed by at least one 600 mg, 900 mg, 1200 mg, 1800 mg, 2400 mg, or 3000 mg subcutaneous dose administered between weeks 8 and 52, preferably every two weeks (q2w).

TABLE 3
Exemplary Maintenance Therapy >8 Weeks-Week 52:

| | Maintenance DOSE (>8 WEEKS) | | | | | |
|---|---|---|---|---|---|---|
| | 600 | 900 | 1200 | 1800 | 2400 | 3000 |
| EACH DOSE | MG | MG | MG | MG | MG | MG |
| FREQUENCY | Q2W, | Q2W, | Q2W, | Q2W, | Q2W, | Q2W/ |
| | Q4W | Q4W | Q4W | Q4W | Q4W | Q4W |
| TIMING (>W 8- | 8, 10, | 8, 10, | 8, 10, | 8, 10, | 8, 10, | 8, 10, |
| W 52/Q2W) | 12 . . . 52 | 12 . . . 52 | 12 . . . 52 | 12 . . . 52 | 12 . . . 52 | 12 . . . 52 |
| (>W 8, Q4W) | 8, 12, | 8, 12, | 8, 12, | 8, 12, | 8, 12, | 8, 12, |
| | 16 . . . 52 | 16 . . . 52 | 16 . . . 52 | 16 . . . 52 | 16 . . . 52 | 16 . . . 52 |
| ADMINISTRATION | SC | SC | SC | SC | SC | SC |

In another embodiment related to any of the above embodiments or aspects first to sixth, the administration results in one or more of the following efficacy endpoints:
(a) Percent change from baseline in dT count at Week 12
(b) Achievement of Hidradenitis Suppurativa Clinical Response (HiSCR) at Week 12.
(c) Absolute change from baseline in International Hidradenitis Suppurativa Severity Score System (IHS4) value at Week 12
(d) Absolute change from baseline in Hidradenitis suppurativa area and severity index (HASI) score at Week 12
(e) Achievement of at least 30% reduction from baseline in Numerical rating scale (NRS30) in Patient's Global Assessment of HS Pain at Week 12
(f) Achievement of Physician global assessment (PGA) score of 0 or 1 at Week 12
(g) Occurrence of complete elimination of dT at Week 12
(h) Occurrence of at least one flare (defined as at least 25% increase in AN count with a minimum increase of 2 relative to baseline up to Week 12.
(i) Absolute change from baseline in Dermatology life quality index (DLQI) Score at Week 12.
(j) Absolute change from baseline in Hidradenitis suppurativa quality of life (HiS-QOL) Total Score at Week 12.

In another embodiment related to any of the above embodiments or aspects first to sixth, the administration results in one or more of the following efficacy endpoints:
(a) Percent change from baseline in dT count at Weeks 8 and/or 16;
(b) Absolute change from baseline in IHS4 value at Week 8 and/or 16;
(c) Absolute change from baseline in Hidradenitis Suppurativa Area Severity Index (HASI) score at Week 16;
(d) Achievement of Hidradenitis Suppurativa Clinical Response (HiSCR50) at Week 16;
(e) Percent change from baseline in abscess count at Week 16;
(f) Achievement of at least a 50% reduction in dT count at Week 16 relative to baseline;
(g) Achievement of at least a 50% reduction in abscess and dT (AdT) count at Week 16 relative to baseline;
(h) Achievement of at least a 50% reduction in ANdT count at Week 16 relative to baseline;
(i) Achievement of at least 30% reduction from baseline in Numerical rating Scale (NRS30) in Patient's Global Assessment of HS pain at Week 16;
(j) Percent change from baseline in dT count at each scheduled assessment;
(k) Achievement of at least 50% reduction from baseline in dT count at each scheduled assessment;
(l) Occurrence of complete elimination of dT at each scheduled assessment;
(m) Time to first occurrence of complete elimination of dT during the treatment period;
(n) Percent change from baseline in abscess count at each scheduled assessment;
(o) Achievement of at least 50% reduction from baseline in abscess count at each scheduled assessment;
(p) Occurrence of complete elimination of abscess at each scheduled assessment;
(q) Percent change from baseline in total AdT count at each scheduled assessment;
(r) Achievement of at least 50% reduction from baseline in AdT count at each scheduled assessment;
(s) Percent change from baseline in inflammatory nodule (N) count at each scheduled assessment;
(t) Achievement of at least 30% reduction from baseline in Numerical rating scale (NRS30) in Patient's Global Assessment of HS pain at each scheduled assessment;
(u) Absolute change from baseline in Hidradenitis Suppurativa Quality of Life (HiS-QoL) total score at each scheduled assessment;
(v) Absolute change from baseline in FACIT-Fatigue scale score at each scheduled assessment;
(w) Absolute change from baseline in Dermatology Life Quality Index (DLQI) score at each scheduled assessment;
(x) Absolute change from baseline in Patient Global Impression of Change (PGI-C) score over time.
(y) Absolute change from baseline in Patient Global Impression of Severity (PGI-S) score over time;
(z) Change from baseline in Hidradenitis Suppurativa odour and drainage scale (HODS) at each scheduled assessment;
(aa) Change from baseline in NRS Pruritus at each scheduled assessment;
(bb) Change from baseline in Hospital Anxiety and Depression Scale (HADS) at each scheduled assessment.
(cc) Percent change from baseline in total ANdT count at each scheduled assessment; or
(dd) Achievement of at least 50% reduction from baseline in ANdT count at each scheduled assessment.

In another embodiment related to above embodiments or aspects first to sixth, the proportion of individuals with a response to the administration is statistically significantly higher as compared to individuals on placebo for one or more of end points (a)-(j) and/or (a)-(dd).

In a seventh aspect, the present invention relates to a method of treating or preventing the recurrence of HS symptoms in a patient treated with one or more parenteral dose(s) of the anti-IL-36R antibody according to any of the preceding aspects and embodiments, said method comprising administering to the patient a therapeutically or prophylactically effective amount of the anti-IL-36R antibody in one or more intravenous or subcutaneous doses.

In an eighth aspect, the present invention relates to a method of achieving a Hidradenitis Suppurativa Clinical Response (HiSCR50) score in a patient treated with one or more parenteral dose(s) of the anti-IL-36R antibody according to any of the preceding aspects and embodiments, said method comprising administering to the patient an effective amount of the anti-IL-36R antibody in one or more intravenous or subcutaneous dose.

In a ninth aspect, the present invention relates a method of achieving a complete resolution of HS symptoms in a patient treated with one or more parenteral dose(s) of the anti-IL-36R antibody according to any of the preceding aspects and embodiments, said method comprising administering to the patient an effective amount of the anti-IL-36R antibody in one or more intravenous or subcutaneous doses wherein the HS symptoms comprise inflammatory lesions, abscesses, draining fistulae/sinus tracts or tunnels (dT), HS associated inflammation (erythema, induration, open ulcers), HS associated infection, and/or HS associated pain.

In a tenth embodiment, the invention provides methods of treatment of hidradenitis suppurativa, including methods of improving symptoms of HS disease, reduction of symptoms of HS disease, and improvements in quality of life for individuals with hidradenitis suppurativa, comprising administering a therapeutically or prophylactically effective amount of the anti-IL-36R antibody according to any of the preceding aspects and embodiments, said method comprising administering to the patient one or more intravenous or subcutaneous doses.

In an eleventh embodiment, the invention provides a method for treating certain subpopulations of individuals, including, for example, those who have failed prior therapy or have had as sub-therapeutic response, including, for example, a subject who has an inadequate response to or is intolerant to, or has a contraindication to, oral antibiotics. In certain embodiments, the invention is used to treat HS in a subject who was unresponsive or intolerant to oral antibiotics for treatment for their hidradenitis suppurativa, or a subject who has an inadequate response to or is intolerant to or has a contraindication to TNF-α inhibitors. In certain embodiments, the invention is used to treat HS in a subject who was unresponsive or intolerant to TNF-α for treatment for their hidradenitis suppurativa.

In a twelfth embodiment, the invention includes the treatment of an individual who has an AN count of greater than or equal to 1 at baseline, greater than or equal to 2 at baseline, greater than or equal to 3 at baseline, greater than or equal to 4 at baseline, or greater than or equal to 5 at baseline.

In another embodiment related to above embodiments or aspects ten to twelve, the invention provides a method for decreasing the number of inflammatory lesions (AN count) or achievement of Hidradenitis suppurativa Clinical Responsiveness (HiSCR) in an individual having HS, said method comprising systemically administering an isolated human anti-IL36R. antibody, or an antigen binding portion thereof, to the subject, such that the AN count is decreased. Clinical Responsiveness according to the HiSCR scoring system is defined as at least a 50% reduction from baseline in the total AN count, with no increase in the abscess or draining fistula count.

In another embodiment related to above embodiments or aspects nine to twelve, the subject has no increase in an abscess count and/or no increase in a draining fistula or tunnel (dT) count following administration with the anti-IL36R antibody, or an antigen binding portion thereof.

In another embodiment related to above embodiments or aspects ten to twelve, the invention provides a method for decreasing the severity of HS according to the IHS4 value, which is the absolute change from baseline according to the International Hidradenitis Suppurativa Severity Score System (IHS4) for the assessment of HS severity. Determining IHS4 requires counting nodules, abscesses, and draining fistulae/sinus tracts. The absolute change from baseline or “IHS4” score assesses the HS severity and the resulting IHS4 score is arrived at by the number of nodules (multiplied by 1) plus the number of abscesses (multiplied by 2) plus the number of draining tunnels (multiplied by 4). A total score of 3 or less signifies mild, 4-10 signifies moderate and 11 or higher signifies severe disease.

In another embodiment related to above embodiments or aspects ten to twelve, the invention provides a method for improving (or reducing) the symptoms of HS as measured by the severity of HS symptoms according to the Hidradenitis Suppurativa Area and Severity Index (HASI) score of a subject having hidradenitis suppurativa from a high score (e.g., an HASI score of 3 or more in one or more regions) to a no or small impact score (e.g., an HS-PGA score of 0-2), comprising administering an IL-36R inhibitor, e.g., human IL-36R. antibody, or antigen-binding portion thereof, to the subject, such that the HASI score improves from the high score to the no or Small impact score. The HASI score is modelled after the Psoriasis Activity and Severity Index (PASI). Four classic signs of HS-related inflammation (erythema, induration, open ulcer, and draining tunnels) are included. Each variable in HASI is scored on a Likert scale (0-3) for each predetermined body region. For BSA assessment, the number of palms (one palm indicated 1% of the patient's BSA) involved for each body region (head, right axilla, left axilla, anterior chest, back, anterior bathing trunk, posterior bathing trunk, other) is assessed. This is converted to a percentage of that region. An area score was assigned to each region using the PASI approach (0=none, 1=1-9%, 2=10-29%, 3=30-49%, 4=50-69%, 5=70-89%, 6=90-100%). To calculate the regional HASI score, the sum of the 4 clinical variable scores is multiplied by the area score of each involved region. This value is then multiplied by the proportion of the BSA of that region, to give a regional HASI score. Regional HASI scores are added together to give the cumulative total HASI score (range 0-72). “HASI” combines the assessment of the severity of lesions and the area affected into a single score in the range 0 (no disease) to 72 (severe disease).

In another embodiment related to above embodiments or aspects ten to twelve, the invention provides a method of improving (i.e., reducing) a Hidradenitis Suppurativa-Physicians Global Assessment (HS-PGA) score. The invention also provides a method of decreasing an HS PGA score of a subject having hidradenitis suppurativa by at least about 2 grades, comprising administering an IL36R inhibitor, e.g., human IL36R. antibody, or antigen-binding fragment thereof. The HS-PGA document is the physician's assessment of the patient's HS at a given time-point. It scores patient disease severity as either clear, minimal, mild, moderate to severe, or very severe, on the basis of abscesses, draining fistulae, inflammatory nodule and non-inflammatory nodule (Kimball A B, Kerdel F, Adams D, et al. Adalimumab for the treatment of moderate to severe hidradenitis suppurativa: a parallel randomized trial. Ann Intern Med 2012; 157:846-855). The “HS-PGA score” ranges from 0 to 5, where 0 (clear: no abscesses, draining tunnels, inflammatory nodules or noninflammatory nodules), 1 (minimal: no abscesses, draining tunnels or inflammatory nodules and the presence of noninflammatory nodules), 2 (mild: no abscesses or draining tunnels and 1-4 inflammatory nodules, or 1 abscess or draining tunnel and no inflammatory nodules), 3 (moderate: no abscesses or draining tunnels and ≥5 inflammatory nodules, or 1 abscess or draining tunnel and ≥1inflammatory nodule, or 2-5 abscesses or draining tunnels and <10 inflammatory nodules), 4 (severe: 2-5 abscesses or draining tunnels and ≥10 inflammatory nodules), 5 (very severe: >5 abscesses or draining tunnels).

In another embodiment related to above embodiments or aspects ten to twelve, the invention provides a method of decreasing an HS-PGA score of a subject having hidradenitis suppurativa from a high score (e.g., a score of 3 or more) to a no or small impact score (e.g., a score of 0-2), comprising administering a human IL-36R antibody to the subject, such that the HS-PGA score is decreased from the high score to the no or small impact score.

In another embodiment related to above embodiments or aspects ten to twelve, the invention provides a method of decreasing an HS-PGA score of a subject having hidradenitis suppurativa by at least about 2 grades, comprising administering a human IL-36R antibody to the subject, such that the HS-PGA score is decreased by at least about 2 grades.

In another embodiment related to above embodiments or aspects ten to twelve, the Numerical rating scale (NRS30) in Patient's Global Assessment of Hidradenitis suppurativa (HS) pain may also be used as an index for measuring efficacy of an anti-IL36R antibody, or an antigen-binding portion thereof in a patient or patient population having hidradenitis suppurativa, where mean statistically significant (as compared to placebo) improvement within a population of treated individuals in their NRS30 Pain score that is at least 30% indicates that the IL-36R antibody is effective for treating hidradenitis suppurativa. The HS Pain NRS is an endpoint for the assessment of HS-related pain severity for clinical trials with individuals with HS. Response was given by an 11-point scale ranging from 0 (no HS pain) to 10 (HS pain as bad as one can imagine). In related embodiment, the invention provides a method for determining whether a human IL-36R. antibody is effective for treating hidradenitis suppurativa based on improvement in NRS30 Pain score.

In another embodiment related to above embodiments or aspects ten to twelve, the invention provides a method of improving a Dermatology Life Quality Index (DLQI) score of a subject having hidradenitis suppurativa from a high score (i.e., the quality of life is impaired) to a low score (i.e., the quality of life not impacted or low impact) comprising administering a human IL-36R antibody to the subject, such that the DLQI score is decreased from the high score to the low score. The DLQI is a patient-administered, ten-question, quality of life questionnaire that covers six domains including symptoms and feelings, daily activities, leisure, work and school, personal relationships and treatment (Finlay A Y, Khan G K. Dermatology Life Quality Index (DLQI)—a simple practical measure for routine clinical use. Joint Ann Mtg of the British Association of Dermatologists and the Canadian Dermatology Association, Oxford, 6-10 Jul. 1993. Clin Exp Dermatol 1994; 19:210-216). The DLQI has a one week recall period. Response categories include “not relevant” (score of 0), “not at all” (score of 0), “a little” (score of 1), “a lot” (score of 2) and “very much” (score of 3). Question 7 is a “yes”/“no” question where “yes” is scored as 3. The DLQI total score is calculated by summing the scores of each question resulting in a range of 0 to 30. The higher the score, the more the quality of life is impaired.

In another embodiment related to above embodiments or aspects ten to twelve, the invention provides a method of improving a Hidradenitis Suppurativa Quality of Life (HiS-QoL) score of a subject having hidradenitis suppurativa from a high score (i.e., the quality of life is highly impacted) to a low score (low impact on the quality of life) comprising administering a human IL-36R antibody to the subject, such that the HiS-QoL score is decreased from the high score to the low score. The HiS-QoL survey is a patient-administered, 17-item instrument to measure HS-specific quality of life in clinical trials. The 17-item HiS-QoL included four symptom items, eight activity-adaptation items and five psychosocial items. The item scores are summed to create a total ranging from 0 to 68, with higher scores indicating more severe impact on quality of life.

In another embodiment related to above embodiments or aspects ten to twelve, the invention provides a method of improving symptoms of HS or reducing pruritus severity related to HS as scored according to the Pruritus Numerical Rating Scale (NRS Pruritus) comprising administering a human IL-36R antibody to the subject, such that the score is decreased from the high baseline score to a low score. The HS Pruritus NRS is an endpoint for the assessment of HS-related pruritus severity for clinical trials with individuals with HS. The Patient Global Assessment of HS Pruritus assess the worst HS pruritus. Ratings range from 0 (no itch) to 10 (HS worst imaginable itch). It is a unidimensional measure of pruritus intensity and can be administered daily with minimal trial participant burden. Recall period is 24 h and response is given by an 11-point scale ranging from 0 (“no itch”) to 10 (“worst imaginable itch”). Trial participants are asked to rate the intensity of their itch using this scale.

In another embodiment related to above embodiments or aspects ten to twelve, the invention provides a method of improving symptoms of HS or reducing the incidence of drainage and odour resulting from clinical symptoms of HS, according to the Hidradenitis Suppurativa Odour and Drainage scale (HODS), comprising administering a human IL-36R antibody to the subject, such that the score is decreased from the high baseline score to a low score. The HODS is an 8-item scale developed to assess the HS-related drainage and odour in individuals. It covers two domains: drainage (5 items) and odour (3 items). The response options range from 1-5. Higher scores depict worse outcome of the concept being assessed.

In another embodiment related to above embodiments or aspects ten to twelve, the invention provides a method of reducing and or ameliorating anxiety and/or depression of individuals with HS according to the Hospital Anxiety and Depression Scale (HADS), comprising administering a human IL-36R antibody to the subject, such that the score is decreased from the high baseline score to a low score. The HADS is an instrument for screening anxiety and depression in non-psychiatric populations. The HADS consists of 14 items, 7 each for anxiety and depression symptoms; possible scores range from 0 to 21 for each subscale.

In another embodiment related to above embodiments or aspects ten to twelve, the invention provides a method of improving the impact of HS on a patient's work productivity, and/or overall activity according to the Work Productivity and Activity Impairment Questionnaire for HS (WPAI-HS) comprising administering a human IL-36R antibody to the subject, such that the score is decreased from the high baseline score (high impact) to a low score (low impact). The WPAI-HS is 6-item instrument to assess the effect of hidradenitis suppurativa on ability to work and perform normal daily activities.

In another embodiment related to above embodiments or aspects ten to twelve, the invention provides a method of decreasing the impact of HS on a patient's quality of life and/or improving a patient's overall quality of life as evaluated by EuroQol 5 dimensions 5-level (EQ-5D-5L), comprising administering a human IL-36R antibody to the subject, wherein an improvement is a change from an high impact score to a low impact score. The descriptive system comprises five dimensions: mobility, self-care, usual activities, pain/discomfort, and anxiety/depression. Each dimension has 5 levels: no problems, slight problems, moderate problems, severe problems, and extreme problems. The EQ VAS records the patient's self-rated health on a vertical visual analogue scale, where the endpoints are labelled ‘The best health you can imagine’ and ‘The worst health you can imagine’.

In another embodiment related to above embodiments or aspects ten to twelve, proportion of individuals with a positive response to the administration of anti-IL36R is statistically and/or significantly higher as compared to individuals on placebo for one or more of end points. The endpoints may be a composite score of multiple scores using various evaluation tools.

In a thirteenth aspect, the present invention relates to a method of treating and/or preventing the recurrence of HS symptoms in a patient treated with one or more parenteral dose(s) of the anti-IL-36R antibody according to any of aspects or the above embodiments, said method including administering to the patient an effective amount of the anti-IL-36R antibody in one or more intravenous or subcutaneous doses.

In fourteenth aspect, the present invention relates to a method of achieving a 50% reduction from baseline in the total AN count, with no increase in the abscess or draining fistula count in a patient treated with one or more dose(s) of the anti-IL-36R antibody according to any of aspects of the above embodiments, said method including administering to the patient an effective amount of the anti-IL-36R antibody in one or more intravenous and/or subcutaneous doses.

In a fifteenth aspect, the present invention relates to a method of achieving improvement of one or more HS symptoms in a patient treated with one or more intravenous dose(s) or subcutaneous dose comprising a loading dose, followed by one or more subcutaneous dose(s) comprising a maintenance dose of the anti-IL-36R antibody according to any of aspects or the above embodiments; wherein the HS symptoms comprise inflammatory lesions, abscesses, draining fistulae/sinus tracts or tunnels (dT), HS associated inflammation (erythema, induration, open ulcers), HS associated infection, and/or HS associated pain.

In one embodiment related to any of aspects above or the related embodiment(s) thirteen to fifteen, at least 10%, 20%, 30%, 40%, 50%, 60%, 70% or 80% of the individuals show significant clinical improvement as measured by percent change from baseline in total AN count at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show significant clinical improvement in the occurrence of HS flare (the incidence of flare defined as at least 25% increase in AN count with a minimum increase of 2 occurrences) relative to baseline total AN count at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show significant clinical improvement as measured by a change in percent change from baseline in total dT count at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show complete elimination of dT at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals achieve clinical responsiveness as measured by Hidradenitis Suppurativa Clinical Response score HiSCR defined as at least a 50% reduction from baseline in the total AN count, with no increase in the abscess or draining fistula count at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show significant clinical improvement as measured by an absolute change from baseline in International Hidradenitis Suppurativa Severity Score system (IHS4) at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show significant clinical improvement as measured by an absolute change from baseline in Hidradenitis Suppurativa Area and Severity Index (HASI) at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show 30% reduction from baseline in Numerical rating scale (NRS30) in Patient's Global Assessment of HS Pain at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show a score of 0 or 1 measured by the Physician Global Assessment (PGA) at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show clinical improvement as measured by absolute change from baseline in the Dermatology life quality index (DLQI) Score at Week 8, 12, 16, 20, 24, 36, 48, 52, 60 or 72 of the treatment.

In one embodiment related to any of aspects above or the related embodiment(s) thirteen to fifteen, at least 10%, 20%, 30%, 40%, 50%, 60%, 70% or 80% of the individuals show clinical improvement as measured by absolute change from baseline in the Hidradenitis suppurative quality of life (HiS-QoL) Total Score at Week 8, 12, 16, 20, 24, 36, 48, 52, 60 or 72 of the treatment.

In one embodiment related to any of aspects above or the related embodiment(s) thirteen to fifteen, the proportion of individuals with a response to the administration is statistically significantly higher as compared to individuals on placebo for any of the end points recited.

In another aspect, the present invention relates to a method of treating HS in a patient, including (a) obtaining a biological sample from said patient, wherein the biological sample is obtained from source including lesional skin or whole blood;
(b) determining the gene express profile of one or more of genes;
(c) administering to the patient an effective amount of the anti-IL-36R antibody according to any embodiments relating to any of the aspects above.

In one embodiment relating to aspect tenth, the one or more of genes are IL12B, IL1B, IL6, CXCL1, IL23A, TNF, IL17C, IL24 or IL1B in lesional skin, and IL1B, S100A9, S100A12, S100A8, MMP25, MMP9 or CD177 in whole blood.

It will be understood that any of the herein disclosed methods, administration schemes and/or dosing regimens also equally apply to the use of any of the disclosed anti-IL-36R antibodies in such methods, administration schemes and/or dosing regimens: i.e., an anti-IL-36R antibody, as disclosed herein, for use in the treatment, prevention, reducing and/or amelioration of any of the disclosed diseases and/or conditions. In other words, the invention also provides for the use of an anti-IL-36R antibody, as disclosed herein, for the manufacture of a medicament for the treatment, prevention, reducing and/or amelioration of any of the disclosed diseases and/or conditions.

Additional features and advantages of the present invention will-become apparent from a review of the ensuing detailed description-set forth below, and in part will be apparent from the description, or may be learned by practice of the subject technology. It is to be understood that both the foregoing general description and the following detailed description are exemplary and explanatory and are intended to provide further explanation of the present invention as claimed.

### BRIEF DESCRIPTION OF THE FIGURES

The accompanying drawings, which are included to provide further understanding of the present invention and are incorporated in and constitute a part of this specification, illustrate aspects of the subject technology and together with the description serve to explain the principles of the present invention.

FIG. 1 shows the IL-36 antagonist ligands (IL-36RA/IL1F5, IL-38/ILF10) inhibiting the signaling cascade.

FIG. 2 shows IL-36 Ligand expression by in situ hybridization (ISH) techniques in human skin biopsies. It shows formalin fixed paraffin embedded (FFPE) skin biopsies from HS lesional and non-lesional HS samples were purchased from a vendor and using ISH probes, stained for IL-36 α,β, γ. Increased expression of as all three IL36 ligands, α, ß γ are seen in HS skin samples.

FIG. 3A-3B A. Phase IIa Clinical Study Design: proof-of-clinical-concept study aimed to explore the effect of spesolimab in individuals with moderate-to-severe HS. Individuals eligible for inclusion also had HS lesions in ≥2 distinct body areas; a total abscess and inflammatory nodule (AN) count≥5; a total draining tunnel count≤20; were biologic-naïve or had failed on previous TNFi treatment for HS and had an inadequate response to oral antibiotics for HS in the past year. †HS disease worsening was defined as a 150% increase in AN count from baseline; rescue monotherapy with either doxycycline 100 mg orally twice daily, or an alternative as per investigator discretion could be given for a maximum of 2 weeks, and not more than a total of 4 weeks over the course of the study. 3B Baseline Patient demographics: ‡HS severity was based on IHS4 criteria. AN, abscess and inflammatory nodule; HS, hidradenitis suppurativa; IHS4, International Hidradenitis Suppurativa Severity Score System; i.v., intravenous; OLE, open-label extension; qw, once weekly; q2w, once every 2 weeks; SC, subcutaneous; SD, standard deviation. TNFi, TNF inhibitor.

FIG. 4. Flowchart of Phase IIa Trial Activities.

FIG. 5A-5B. A. LS mean percentage change from baseline in total abscess and inflammatory nodule (AN) count: LS means, differences and confidence intervals were estimated by (REML)-based MMRM including the fixed, categorical effects of treatment at each visit, prior use of TNF inhibitor strata and the continuous effect of baseline at each visit as well as random effects of subject. Analyses use data up to the use of rescue therapy; data after the use of rescue therapy are censored. Results are presented descriptively; individuals with non-missing values are included in the summary. B. Mean absolute change from baseline in lesion counts at week 12: Counts for all types of HS inflammatory lesions tended to decrease after treatment with spesolimab. AN, abscess and inflammatory nodule; dT, draining tunnel; LS, least squares; SD, standard deviation; SE, standard error.

FIG. 6A-6B. A. Percentage change from baseline in dT count at Week 12. A greater proportion of individuals in the spesolimab vs. the placebo arm had a decrease from baseline in dT at Week 12. 66.7% of individuals in the spesolimab arm (16/24) vs 38.5% of individuals in the placebo arm (5/13) had a decrease from baseline in dT at Week 12. B. LS mean percentage change from baseline in dT count: LS means, differences and confidence intervals were estimated by (REML)-based MMRM including the fixed, categorical effects of treatment at each visit, prior use of TNF inhibitor strata and the continuous effect of baseline at each visit as well as random effects of subject. Analyses use data up to the use of rescue therapy; data after the use of rescue therapy are censored. dT, draining tunnel; LS, least squares; SE, standard error.

FIG. 7. The safety profile of spesolimab: The safety profile was similar to previous trials; no patient receiving spesolimab had a serious adverse event. At Week 2, two individuals received inverted treatment, therefore, 36 individuals were exposed to spesolimab. ^{†}Severe AEs were those with an RCTC Grade of 3 or 4. ^{‡}According to ICH E3 guidelines. AE, adverse event; AESI, adverse event of special interest; AN, abscess and inflammatory nodule; HS, hidradenitis suppurativa; N, nodule; RCTC, Rheumatology Common Toxicity Criteria.

FIG. 8A-8B. A. Phase IIa and OLE Study Design: ^{†}Week 24 (Week 12 of the OLE), individuals could have their dose of spesolimab increased to 1200 mg SC q2w if required (based on responder status at start of OLE and HS-PGA severity change from start of OLE); ^{‡}HS disease worsening was defined as a 150% increase in abscess and inflammatory nodule count from baseline; rescue monotherapy with systemic antibiotics or immunosuppressive biologics; ^{§} Baseline refers to last measurement before start of spesolimab, i.e. baseline of Phase IIa trial in prior spesolimab arm or baseline of OLE study in prior placebo arm (IV, intravenous; OLE, open-label extension; q2w, once every 2 weeks; R, randomized; SC, subcutaneous; SD, standard deviation) B. Patient characteristics at OLE baseline: Individuals eligible for inclusion also had HS lesions in ≥2 distinct body areas; a total abscess and inflammatory nodule (AN) count≥5; a total draining tunnel count≤20; were biologic-naïve or had failed on previous TNF-α inhibitor treatment for HS and had an inadequate response to oral antibiotics for HS in the past year. (DLQI, Dermatology Life Quality Index; HASI, Hidradenitis Suppurativa Area and Severity Index; HIS-QOL, Hidradenitis Suppurativa Quality of Life; HS, hidradenitis suppurativa; HS-PGA, Hidradenitis Suppurativa Physician Global Assessment; IHS4, International Hidradenitis Suppurativa Severity Score System)

FIG. 9A-9D. Absolute change from baseline to Week 50 for lesion types and IHS4 score: 95% CIs were calculated based on Student's t-distribution. Patient numbers at baseline were prior spesolimab n=30 and prior placebo n=15. For change from baseline, only individuals with ≥1 relevant lesion type were included. Patient numbers at Week 50 were prior spesolimab n=15 and prior placebo n=7. (All assessments after rescue medication are excluded. CI, confidence interval; IHS4, International Hidradenitis Suppurativa Severity Score System). A. Mean (95% CI) absolute change from baseline in draining tunnel count. Prior spesolimab group=−1.3 (−2.9, 0.3); Prior placebo group=−3.7 (0.3, −7.8) (Only individuals with ≥1 draining tunnel at baseline are included). B. Mean (95% CI) absolute change from baseline in inflammatory nodule count. Prior spesolimab group=−3.7 (−7.9, 0.5); Prior placebo group=−5.7 (−8.7, −2.8) (Only individuals with ≥1 inflammatory nodule at baseline are included). C. Mean (95% CI) absolute change from baseline in abscess count. Prior spesolimab group=−0.9 (−2.2, 0.5); Prior placebo group=−2.4 (−7.6, 2.8) (Only individuals with ≥1 abscess at baseline are included). D. Mean (95% CI) absolute change from baseline in IHS4 score. Prior spesolimab group=−12.8 (−20.7, −5.0); Prior placebo group=−23.4 (−50.8, 4.0).

FIG. 10A-10B. A. Part I (Phase IIb) Trial Design. multi-center, double-blind, placebo-controlled, randomised trial assessing the efficacy and safety of spesolimab versus placebo in individuals with moderate to severe HS. B. Part 2 (Phase III) Trial Design: starts after primary analysis of Part 1 (Phase IIb), assessment of further development benefit, interaction with HAs, and approval by IRB/EC. (IC=Informed Consent, V=Study Visit, W=Week (study weeks), FUP=Follow Up, EoT=End-of-Treatment, EoS=End-of-Study, REP=Residual Effect Period).

FIGS. 11A & 11B. Flowchart of Phase IIb Trial Activities. A. Treatment period up to week 32. B. Treatment period after week 32 up to week 48.

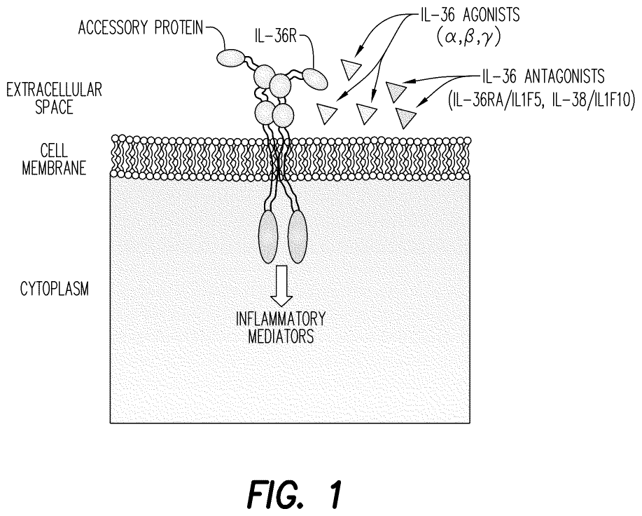

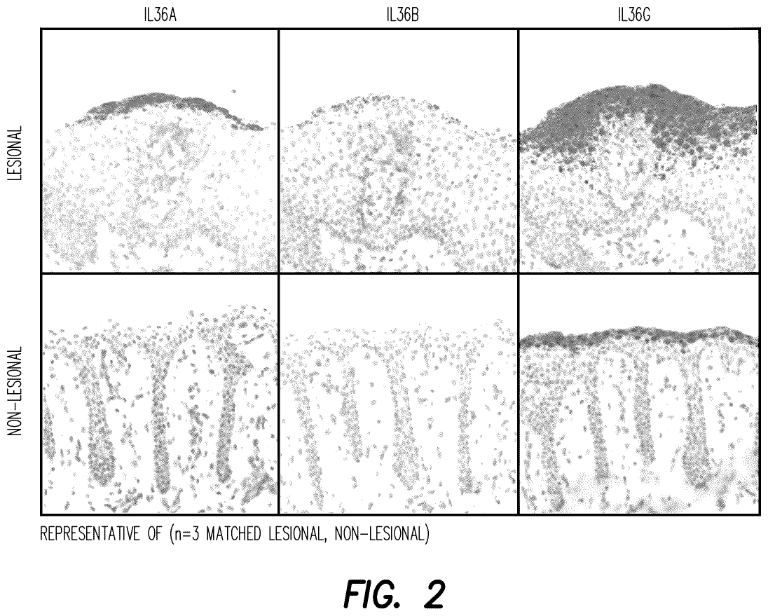

### DETAILED DESCRIPTION OF THE INVENTION

Before the present invention is described, it is to be understood that this invention is not limited to particular methods and experimental conditions described, as such methods and conditions may vary. It is also to be understood that the terminology used herein is for the purpose of describing particular embodiments only, and is not intended to be limiting, since the scope of the present invention will be limited only by the appended claims.

In the following detailed description, numerous specific details are set forth to provide a full understanding of the present invention. It will be apparent, however, to one of ordinarily skilled in the art that the subject technology may be practiced without some of these specific details. In other instances, well-known structures and techniques have not been shown in detail so as not to obscure the present invention.

Unless defined otherwise, all technical and scientific terms used herein have the same meaning as commonly understood by one of ordinary skill in the art to which this invention belongs.

The inventors have surprisingly discovered inter alia that the interleukin-36 pathway was inhibited in HS individuals treated with a high loading dose of an anti-interleukin-36R (anti-IL-36R) monoclonal antibody of the present invention. The resulting positive key indications of responsiveness included: a percent change from baseline in dT count, an absolute change from baseline in IHS4 (dT, abscess, and inflammatory weighed counts), and an absolute change from baseline in HASI (severity index of HS) as well as other clinical symptoms in individuals at 12 weeks after administration.

In one aspect, the present invention relates to a method for treating, preventing or ameliorating hidradenitis suppurativa (HS) in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab.

Without wishing to be bound by this theory it is believed that anti-IL-36R antibodies or antigen-binding fragments thereof bind to human IL-36R and thus interfere with the binding of IL-36 agonists, and in doing so block at least partially the signaling cascade from the IL-36R to inflammatory mediators involved in hidradenitis suppurativa (HS). IL-36R is also known as IL-1RL2 and IL-1Rrp2. It has been reported that agonistic IL-36 ligands (α, β, or γ) initiate the signaling cascade by engaging the IL-36 receptor which then forms a heterodimer with the IL-1 receptor accessory protein (IL-1RAcP).

IL36R is a cell surface receptor involved in inflammatory responses in skin and gut. It is a novel member of the IL1R family that forms a heterodimeric complex with the IL1R accessory protein. The heterodimeric IL36R system with stimulating (IL36a, IL36B, IL36Y) and inhibitory ligands (IL36Ra) shares a number of structural and functional similarities to other members of the IL1/IL1R family, such as IL1, IL18 and IL33 (R17-3602). All IL1 family members (IL1α, IL1β, IL18, IL36α, IL36β, IL36γ, and IL38) signal through a unique, cognate receptor protein which, upon ligand binding, recruits the common IL1RacP subunit and activates NFkB and MAP kinase pathways in receptor-positive cell types. In human skin tissues, IL36R is expressed in keratinocytes, dermal fibroblasts and infiltrating myeloid cells. IL36R activation in skin tissue drives the production of inflammatory mediators (e.g., CCL20, MIP-1β, TNF-α, IL12, IL17, IL23, TGF-β) and modulates the expression of tissue remodeling genes (e.g., MMPs, TGF-β).

The anti-IL36R antibodies of the present invention are disclosed herein an in, for example, in U.S. Pat. No. 9,023,995, the entire content of which is incorporated herein by reference.

Although any methods and materials similar or equivalent to those described herein can be used in the practice of the present invention, the preferred methods and materials are now described. All publications mentioned herein are incorporated herein by reference to describe in their entirety.

### Definitions

A phrase such as “an aspect” does not imply that such aspect is essential to the present invention or that such aspect applies to all configurations of the subject technology. A disclosure relating to an aspect may apply to all configurations, or one or more configurations. An aspect may provide one or more examples of the disclosure. A phrase such as “an aspect” may refer to one or more aspects and vice versa. A phrase such as “an embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples of the disclosure.

The term “about” shall generally mean an acceptable degree of error or variation for the quantity measured given the nature or precision of the measurements. Typical, exemplary degrees of error or variation are within 5% or within 3% or within 1% of a given value or range of values. For example, the expression of “about 100” includes 105 and 95 or 103 and 97 or 101 and 99, and all values in between (e.g., 95.1, 95.2, etc. for range of 95-105; or 97.1, 97.2, etc. for the range of 97-103; 99.1, 99.2, etc. for the range of 99-101). Numerical quantities given herein are approximates unless stated otherwise, meaning that the term “about” can be inferred when not expressly stated.

### Therapeutic Uses of the Antibodies

Increasingly, the role of neutrophils in skin inflammation is being understood. While the role of IL-36 pathway based on genetics and proven inhibition with spesolimab is clear, the involvement of the same pathway in other similar neutrophilic dermatoses (NDs), which are a heterogeneous set of conditions with common features and overlapping pathophysiology, is being appreciated. While the disease is associated primarily with cutaneous manifestations due to accumulation of neutrophils, the involvement of additional tissues is also seen. Our studies suggest that the IL-36 pathway plays an important role in driving disease manifestations in pustular neutrophilic dermatoses (NDs) such as hidradenitis suppurativa (HS). Accordingly, blocking the IL-36 pathway should be beneficial in skin inflammation with papules, nodules and plaques such as hidradenitis suppurativa (HS).

The present provides anti-IL-36R antibodies and a pharmaceutical composition comprising an anti-IL-36R antibody for use in reducing, diminishing or blocking the IL-36 pathway which is useful for the treatment or prevention of hidradenitis suppurativa (HS) in subjects, preferably humans.

In one embodiment, the invention provides a pharmaceutical composition formulated in a unit dose form wherein such single dose form comprises at least 300 mg, 450 mg, 600 mg, 900 mg, 1200 mg, 1800 mg, 2000 mg, 2400 or 3000 mg of said anti-IL-36R antibody. In a related embodiment, the unit dose form is used in the preparation of a medicament for treating a subject having hidradenitis suppurativa (HS). In a related embodiment, the unit dose form is a parenteral (e.g. intravenous or subcutaneous) dose form.

In a related embodiment, the invention provides a drug preparation (or “article of manufacture” described hereinafter), which comprises one, two, three or four said unit dose forms comprising 300 mg of said anti-IL-36R antibody; one, two, three or four said unit dose forms comprising 450 mg of said anti-IL-36R antibody; or one, two, three or four said unit dose forms comprising 600 mg of said anti-IL-36R antibody; or one, two, three or four said unit dose forms comprising 900 mg of said anti-IL-36R antibody; or one, two, three or four said unit dose forms comprising 1200 mg of said anti-IL-36R antibody; or one, two, three or four said unit dose forms comprising 1800 mg of said anti-IL-36R antibody; or one, two, three or four said unit dose forms comprising 2000 mg of said anti-IL-36R antibody; or one or two said unit dose forms comprising 2400 mg of said anti-IL-36R antibody; or one or two said unit dose forms comprising 3000 mg of said anti-IL-36R antibody; or any combination thereof to achieve an efficacious dose.

In a related embodiment, the one, two, three or four said unit dose forms (e.g., in the drug preparation) are administered at 1 (qw), 2 (q2w), or 4-week (q4w) intervals. Preferably, when the unit dose forms comprises 450 mg, 600 mg, 900 mg, 1200 mg, 1800 mg, or 2000 mg of said anti-IL-36R antibody, the one, two, three, or four unit dose forms are administered at 1 (qw), 2 (q2w), or 4-week (q4w) intervals; when the unit dose forms comprises 2400 mg or 3000 mg of said anti-IL-36R antibody, the one or two unit dose forms are administered at 2 (q2w) or 4-week (q4w) intervals; when the unit dose forms comprises 300 mg of said anti-IL-36R antibody, the one, two, three, or four unit dose forms are administered at 1 (qw) or 2-week (q2w) intervals as subcutaneous maintenance dose.

In one aspect, the present invention relates to a method for treating, preventing or ameliorating a hidradenitis suppurativa (HS) in a subject, comprising administering to the subject a therapeutically effective amount of an anti-IL-36R antibody or an antigen-binding fragment thereof (as disclosed herein). In an embodiment relating to this aspect, the anti-IL-36R antibody is spesolimab.

In one aspect, the present invention relates to use of an anti-IL-36R antibody or an antigen-binding fragment thereof (as disclosed herein) in the preparation of a medicament for treating, preventing or ameliorating hidradenitis suppurativa (HS). In an embodiment relating to this aspect, the anti-IL-36R antibody is spesolimab.

Preferably, said HS is moderate to severe HS. The “moderate to severe HS” can be determined based on International Hidradenitis Suppurativa Severity Score System (IHS4) criteria, for at least 1 year prior to the baseline visit, as determined by the investigator through participant interview and/or review of the medical history; if IHS4 scoring is not available, equivalent scoring based on scoring systems as HS-PGA or Hurley are acceptable based on documented investigator assessment. More preferably, the HS lesions exist in at least 2 distinct anatomic area (e.g., right/left axillary, inguinal, inframammary, perineal). More preferably, the HS is biologic naïve, previous treatment with a TNF inhibitor (TNFi) for the HS failed. More preferably, when the HS is biologic naïve, inadequate response to an adequate course of appropriate oral antibiotics for treatment of HS occurred in the last 1 year, as per investigator discretion.

In one aspect, the present invention relates to a method of treating a skin disorder associated with hidradenitis suppurativa (HS) in a patient, said method(s) including administering or having administered to the patient a therapeutically effective amount of an anti-IL-36R antibody of the present invention or an antigen binding fragment thereof (as disclosed herein). In an embodiment relating to this aspect, the anti-IL-36R antibody is spesolimab.

In one aspect, the present invention relates to a method of treating skin inflammation associated with hidradenitis suppurativa (HS) in a subject, said method including administering or having administered to the subject a therapeutically effective amount of an anti-IL-36R antibody of the present invention or an antigen binding fragment thereof (as disclosed herein). In an embodiment relating to this aspect, the anti-IL-36R antibody is spesolimab.

In one aspect, the present invention relates to a method preventing or reducing neutrophilic infiltrates in affected skin tissues in a subject suffering from a hidradenitis suppurativa (HS), said method including administering or having administered to the subject a therapeutically effective amount of an anti-IL-36R antibody of the present invention or an antigen binding fragment thereof. In an embodiment relating to this aspect, the anti-IL-36R antibody is spesolimab.

An infectious process related to HS has been suspected for a long time (Highet A S, Warren R E, Staughton R C, Roberts S O. Streptococcus milleri causing treatable infection in perineal hidradenitis suppurativa. Br J Dermatol. 1980; 103:375-82). For example, in one study anaerobic bacteria, such as Prevotella and Porphyromonas, coagulase-negative Staphylococcus, and Staphylococcus aureus were commonly identified microorganisms in HS lesions (Wark K J L, Cains G D. The Microbiome in Hidradenitis Suppurativa: A Review. Dermatol Ther (Heidelb). 2021 February; 11(1):39-52).

The microbiome of tunnels in hidradenitis suppurativa individuals has also been investigated, and overall, five microbiome types were identified: Porphyromonas spp. (type I), Corynebacterium spp., (type II), Staphylococcus spp. (type III), Prevotella spp. (type IV) and Acinetobacter spp (type V). Porphyromonas spp. (type I) and Prevotella spp. (IV) were the most frequent genera found the tunnels (Ring H C, et al. The Follicular Skin Microbiome in Individuals With Hidradenitis Suppurativa and Healthy Controls. JAMA Dermatol. 2017 Sep. 1; 153(9):897-905).

In one study, bacterial cultures detected anaerobes in 83% of HS lesions vs 53% of control samples, combined with milleri group streptococci and actinomycetes in 33% and 26% of cases, respectively. High-throughput sequencing identified 43 taxa associated with HS lesions. Two gram-negative anaerobic rod taxa, Prevotella and Porphyromonas, predominated, contrasting with a reduced abundance of aerobic commensals. The rare taxa of normal skinfold microbiota were associated with lesions independently of gender, duration and familial history of HS, body mass index, and location. Two main additional taxa, Fusobacterium and Parvimonas, correlated with the clinical severity of HS (Guet-Revillet H, Jais J P, Ungeheuer M N, Coignard-Biehler H, Duchatelet S, Delage M, Lam T, Hovnanian A, Lortholary O, Nassif X, Nassif A, Join-Lambert O. The Microbiological Landscape of Anaerobic Infections in Hidradenitis Suppurativa: A Prospective Metagenomic Study. Clin Infect Dis. 2017 Jul. 15; 65(2):282-291).

In Guet-Revillet, et al., 2014, researchers linked HS disease severity with two different microbiological profiles associated with the clinical staging of legions according to the Hurley scale (Guet-Revillet, H., Coignard-Biehler, H., Jais, J., Quesne, G., Frapy, E., Poirée, S., et al. (2014). Bacterial Pathogens Associated with Hidradenitis Suppurativa, France. Emerging Infectious Diseases, 20(12), 1990-1998). While there is no consensus for a grading system, the Hurley staging system is most commonly used (Hurley H J. Axillary hyperhidrosis, apocrine bromhidrosis, hidradenitis suppurativa, and familial benign pemphigus: surgical approach. In: Roenigk R K, Roenigk H H Jr, eds. Roenigk and Roenigk's Dermatologic Surgery: Principles and Practice. 2nd ed. New York, NY: Marcel Dekker; 1996:623-645). Very briefly, the Hurley system divides HS into three stages: stage 1: single or multiple abscesses without sinus tract formation or scarring, which is most common; stage 2: recurrent abscesses with one or more sinus tracts and scarring widely separated by normal skin; and stage 3: diffuse involvement with multiple sinus tracts and no intervening normal skin.

In Guet-Revillet, et al., 2014 researchers demonstrated that 2 specific microbiological profiles, neither corresponding to the normal skin microflora, nor to usual skin pathogens (S. aureus and S. pyogenes) are associated with HS lesions and disease severity. The first microbiological profile “Profile A” was characterized by pure or predominant culture of S. lugdunensis that was mostly associated with Hurley stage 1 lesions (lower severity score). The second microbiological profile “Profile B” was represented by a mixed flora composed of gram-negative and Gram-positive strict anaerobes, anaerobic actinomycetes, and streptococci of the milleri group. Various other bacteria, such as S. aureus, coagulase negative staphylococci, corynebacteria, Enterobacteriaceae, Propionibacterium spp., and Enterococcus spp., were inconstantly and in smaller amounts associated with the mixed anaerobic flora, especially when lesions were sampled by swabbing. This profile was mainly associated with open suppurating lesions observed in Hurley stages 2 and 3, but also with 24% of Hurley stage 1 lesions.

In one aspect, the present invention relates to a method of reducing microbial colonization of the skin in a subject with hidradenitis suppurativa (HS) comprising administering to the subject a therapeutically effective amount of an anti-IL-36R antibody or an antigen-binding fragment thereof, as disclosed herein. In an embodiment relating to this aspect, the colonization is of a microbe selected from the group consisting of Staphylococcus aureus, Streptococcus spp., Pseudomonas aeruginosa, Bacteroides spp., molluscum contagiosum virus, Herpes simplex virus, coxsackievirus, vaccinia virus, Candida albicans, Microsporum spp., Trichophyton spp., Penicillium spp., Cladosporium spp., Alternaria spp., and Aspergillus spp. In another embodiment relating to this aspect, the microbe is Staphylococcus aureus (S. aureus). In another embodiment relating to this aspect, the S. aureus colonization is reduced by at least 10% or by at least 20% from the baseline following the administration of the anti-IL-36R antibody or an antigen-binding fragment thereof, as disclosed herein. In an embodiment relating to this aspect, the anti-IL-36R antibody is spesolimab.

In one aspect, the present invention relates to a method of reducing susceptibility to a skin infection in a subject with hidradenitis suppurativa (HS) comprising administering to the subject a therapeutically effective amount of an anti-IL-36-R antibody or an antigen-binding fragment thereof (as disclosed herein). In an embodiment relating to this aspect, the skin infection is caused by a microbe selected from the group consisting of Staphylococcus aureus, Streptococcus spp., Pseudomonas aeruginosa, Bacteroides spp., Herpes simplex virus, molluscum contagiosum virus, coxsackievirus, vaccinia virus, Candida albicans, Microsporum spp., Trichophyton spp., Penicillium spp., Cladosporium spp., Alternaria spp., and Aspergillus spp. In another embodiment relating to this aspect, the microbe is Staphylococcus aureus (S. aureus). In an embodiment relating to this aspect, the anti-IL-36R antibody is spesolimab.

In an embodiment relating to any of the above aspects, a second therapeutic agent is administered to the subject before, after, or concurrent with the anti-IL-36R antibody or an antigen-binding fragment thereof. In a related embodiment, the second therapeutic agent is selected from the group consisting of an anti-bacterial agent, an anti-viral agent, an anti-fungal agent, another IL-36R antagonist, an IgE inhibitor, a corticosteroid, NSAID, an IL-4R antagonist, and IFNγ.

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
i. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
ii. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iii. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iv. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
v. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
vi. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
vii. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

In an embodiment relating to any of the above aspects, the anti-IL-36R antibody includes:
i. a light chain variable region comprising the amino acid sequence of SEQ ID NO: 77; and a heavy chain variable region comprising the amino acid sequence of SEQ ID NO: 87; or
ii. a light chain variable region comprising the amino acid sequence of SEQ ID NO: 77; and a heavy chain variable region comprising the amino acid sequence of SEQ ID NO: 88; or
iii. a light chain variable region comprising the amino acid sequence of SEQ ID NO: 77; and a heavy chain variable region comprising the amino acid sequence of SEQ ID NO: 89; or
iv. a light chain variable region comprising the amino acid sequence of SEQ ID NO: 80; and a heavy chain variable region comprising the amino acid sequence of SEQ ID NO: 87; or
v. a light chain variable region comprising the amino acid sequence of SEQ ID NO: 80; and a heavy chain variable region comprising the amino acid sequence of SEQ ID NO: 88; or
vi. a light chain variable region comprising the amino acid sequence of SEQ ID NO: 80; and a heavy chain variable region comprising the amino acid sequence of SEQ ID NO: 89; or
vii. a light chain variable region comprising the amino acid sequence of SEQ ID NO: 85; and a heavy chain variable region comprising the amino acid sequence of SEQ ID NO: 100; or
viii. a light chain variable region comprising the amino acid sequence of SEQ ID NO: 85; and a heavy chain variable region comprising the amino acid sequence of SEQ ID NO:101; or
ix. a light chain variable region comprising the amino acid sequence of SEQ ID NO: 86; and a heavy chain variable region comprising the amino acid sequence of SEQ ID NO: 100; or
x. a light chain variable region comprising the amino acid sequence of SEQ ID NO: 86; and a heavy chain variable region comprising the amino acid sequence of SEQ ID NO:101.

In an embodiment relating to any of the above aspects, the anti-IL-36R antibody includes:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In another embodiment relating to any of the above aspects or embodiments described herein, the anti-IL-36R antibody is spesolimab.

In another embodiment relating to any of the above aspects or embodiments described herein, a second therapeutic agent is administered to the subject before, after, or concurrent with the anti-IL-36R antibody.

In another embodiment relating to any of the above aspects or embodiments described herein, the second therapeutic agent is selected from the group consisting of an anti-bacterial agent, an anti-viral agent, an anti-fungal agent, an anti-IL-36R antibody, an IgE inhibitor, a corticosteroid, a non-steroid anti-inflammatory drug (NSAID), an IL-4R antagonist, and IFN-γ.

### Pharmaceutical Composition

An antibody of the invention can be incorporated into pharmaceutical compositions suitable for administration to a subject. The compounds of the invention may be administered alone or in combination with a pharmaceutically acceptable carrier, diluent, and/or excipients, in single or multiple doses. The pharmaceutical compositions for administration are designed to be appropriate for the selected mode of administration, and pharmaceutically acceptable diluents, carrier, and/or excipients such as dispersing agents, buffers, surfactants, preservatives, solubilizing agents, isotonicity agents, stabilizing agents and the like are used as appropriate. Said compositions are designed in accordance with conventional techniques as in e.g., Remington, The Science and Practice of Pharmacy, 19th Edition, Gennaro, Ed., Mack Publishing Co., Easton, P A 1995 which provides a compendium of formulation techniques as are generally known to practitioners.

A pharmaceutical composition comprising an anti-IL-36R monoclonal antibody of the present invention can be administered to a subject suffering from a neutrophilic dermatosis as described herein using standard administration techniques including oral, intravenous, intraperitoneal, subcutaneous, pulmonary, transdermal, intramuscular, intranasal, buccal, sublingual, or suppository administration.

The route of administration of an antibody of the present invention may be oral, parenteral, by inhalation, or topical. Preferably, the antibodies of the invention can be incorporated into a pharmaceutical composition suitable for parenteral administration. The term parenteral as used herein includes intravenous, intramuscular, subcutaneous, rectal, vaginal, or intraperitoneal administration. Peripheral systemic delivery by intravenous or intraperitoneal or subcutaneous injection or infusion is preferred. Suitable vehicles for such injections are known in the art.

The pharmaceutical composition typically must be sterile and stable under the conditions of manufacture arid storage in the container provided, including e.g., a sealed vial or syringe (e.g., to hold the unit dose form). Therefore, pharmaceutical compositions may be sterile filtered after making the formulation, or otherwise made microbiologically acceptable. A typical composition for intravenous infusion could have a volume as much as 250-1000 ml of fluid, such as sterile Ringer's solution, physiological saline, dextrose solution and Hank's solution and a therapeutically effective dose, (e.g., 1 to 100 mg/mL or more) of antibody concentration. Dose may vary depending on the type and severity of the disease. As is well known in the medical arts, dosages for any one subject depends upon many factors, including the patient's size, body surface area, age, the particular compound to be administered, sex, time and route of administration, general health, and other drugs being administered concurrently. A typical dose can be, for example, in the range of 0.001 to 1000 mg; however, doses below or above this exemplary range are envisioned, especially considering the aforementioned factors.

The term “dose,” as used herein, refers to an amount of an anti-IL-36R antibody, or an antigen-binding portion thereof which is administered to a subject.

The term “dosing,” as used herein, refers to the administration of an anti-IL-36R antibody, or an antigen-binding portion thereof to achieve a therapeutic objective (e.g., treatment of hidradenitis suppurativa).

A “dosing regimen” describes a treatment schedule for an anti-IL-36R antibody, or an antigen-binding portion thereof e.g., a treatment schedule over a prolonged period of time or throughout the course of treatment, e.g. administering a first dose of an anti-IL-36R antibody, or an antigen-binding portion thereof at week 0 followed by a second, third, and/or fourth dose of an anti-IL-36R antibody, or an antigen-binding portion thereof, on a weekly or biweekly dosing regimen.

The term “multiple-variable dose” includes different doses of an anti-IL-36R antibody, or an antigen-binding portion thereof which are administered to a subject for therapeutic treatment. “Multiple-variable dose regimen” or “multiple-variable dose therapy” describes a treatment schedule which is based on administering different amounts of an anti-IL-36R antibody, or an antigen-binding portion thereof at various time points throughout the course of treatment.

As used herein, the terms “intravenous dose” (iv), “subcutaneous dose” (sc) refer to the route of administration of the anti-IL-36R antibody. A “loading dose” can be an intravenous dose or subcutaneous dose administered at the beginning of the treatment regimen (also referred to as the “baseline dose”); it may also be referred to as an “initial dose” or “induction dose.” The “maintenance dose” is usually a subcutaneous dose which is administered after the loading dose and/or baseline dose, which may also be referred to as a “subsequent dose”. The loading and maintenance intravenous and subcutaneous doses may all contain the same amount of anti-IL-36R antibody, or an antigen binding fragment thereof, but generally may differ from one another in terms of the amount of the antibody administered or the frequency of administration. In an embodiment, the loading dose is equal or larger than the maintenance dose. A “loading dose” which may be interchangeably referred to as an “initial dose” or “induction dose” can be a single dose or, alternatively, a set of doses comprising a “total loading dose”. The maintenance dose which may also be referred to as a “subsequent dose” can be a single dose or, alternatively, a set of doses for administration.

In certain embodiments, the amount of the anti-IL-36R antibody contained in the induction/initial/loading and maintenance/subsequent doses varies from one another during the course of treatment. In certain embodiments, the one or more initial/induction doses each comprise a first amount of the antibody or antigen-binding fragment thereof and the one or more maintenance/subsequent doses each comprise a second amount of the antibody or antigen-binding fragment thereof. In some embodiments, the first total loading dose of antibody or fragment thereof is 1.5×, 2×, 2.5×, 3×, 3.5×, 4×, or 5× the second or subsequent amount of the antibody or antigen-binding fragment thereof. In certain embodiments, one or more (e.g., 1, 2, 3, 4, or 5 or more) initial doses are administered at the beginning of the treatment regimen as “induction doses” or “loading doses” followed by subsequent doses that are administered as “maintenance doses” or “treatment doses”. In one embodiment, a total loading or induction dose comprises 1800 mg, 2400 mg, 3600 mg, 4800 mg, 6000 mg, 7200 mg, or 8000 mg of the anti-IL-36R antibody administered as one or more intravenous or subcutaneous doses of 450 mg, 600 mg, 900 mg, 1200 mg, 1800 mg, 2000 mg, 2400 mg, or 3000 mg every 1, 2 or 4 weeks (qw, q2w, q4w) between DO and 4 weeks. In one exemplary embodiment, following the loading dose, a maintenance dose or subsequent dose of 1200 mg, 1800 mg, 2400 mg, 3600 mg, 4800 mg, or 6000 mg, is administered as at least one subcutaneous dose(s) of 300 mg, 450 mg, 600 mg, 900 mg, 1200 mg, 2400 mg, or 3000 mg every 1, 2, or 4 weeks (qw, q2w, q4w) between week 4 and week 8. In another embodiment, the subcutaneous dose or maintenance or subsequent dose is administered at least one week following the last loading or induction dose.

The term “treatment phase” or “maintenance phase,” as used herein, refers to a period of treatment comprising administration of an anti-IL-36R antibody, or an antigen-binding portion thereof to a subject in order to maintain a desired therapeutic effect, e.g., improved symptoms associated with hidradenitis suppurativa, and will generally be a period of time starting 4 weeks following the initial loading doses (administered between 0-4 weeks).

The term “maintenance dose” or “treatment dose” is the amount of an anti-IL-36R antibody, or an antigen-binding portion thereof taken by a subject to maintain or continue a desired therapeutic effect. A maintenance dose can be a single dose or, alternatively, a set of doses. A maintenance dose is administered during the treatment or maintenance phase of therapy. In one embodiment, the total maintenance dose is smaller than the induction dose, wherein each dose may be equal to each other when administered in succession. In one embodiment, the invention provides a total maintenance dose of at least 1200, 1800 mg, 2400 mg, 3600 mg, 4800 mg, or 6000 mg administered as one or more subcutaneous doses of 300 mg, 450 mg, 600 mg, 900 mg, 1200 mg, 1800 mg, 2400 mg, or 3000 mg of an anti-IL-36R antibody, or an antigen-binding portion thereof to a subject every 1, 2 or 4 weeks (qw, q2w, q4w). In one embodiment, the maintenance dose is administered every week or every other week beginning 1 or 2 weeks after the last loading dose. In one embodiment, a maintenance dose is administered about 4 weeks following the initial loading dose. The maintenance dose can be up-titrated or down-titrated to achieve the desired therapeutic goal. In preferred embodiments the maintenance dose is smaller than the loading dose and/or in less frequency.

The term “maintenance therapy” or “maintenance dosing regimen” refers to a treatment schedule for a subject or patient diagnosed with a disorder/disease, e.g., hidradenitis suppurativa, to enable them to maintain their health in a given state, e.g., reduced number of inflammatory lesions or achieving a clinical response. In one embodiment, a maintenance therapy of the invention is used for a subject or patient diagnosed with a disorder/disease, e.g., hidradenitis suppurativa to enable them to maintain their health in a state which is completely free of symptoms or a reduction in symptoms associated with the disease. In one embodiment, a maintenance therapy of the invention is used for a subject or patient diagnosed with a disorder/disease, e.g., hidradenitis suppurativa, to enable them to maintain their health in a state which is substantially free of symptoms associated with the disease. In one embodiment, a maintenance therapy of the invention is used for a subject or patient diagnosed with a disorder/disease, e.g., hidradenitis suppurativa, to enable them to maintain their health in a state where there is a significant reduction in symptoms associated with the disease and will generally be a period of time starting 8 weeks following the initial loading doses (administered at 0-4 weeks), and treatment doses (administered between 4-8 weeks). In one embodiment the anti-IL-36R antibody maintenance therapy can be continued after 8 weeks comprising at least one subcutaneous dose(s), wherein each dose is 600 mg, 900 mg, 1200 mg, 1800 mg, 2400 mg, or 3000 mg, administered every 2 (q2w) or 4 (q4w) weeks following the last subcutaneous maintenance dose (e.g. at between 4-8 weeks). In non-limiting, exemplary embodiments, a total maintenance dose of at least 1800 mg is followed by maintenance therapy comprising at least one 600 mg, 900 mg, 1200 mg, 1800 mg, 2400 mg, or 3000 mg subcutaneous dose administered between weeks 8 and 52, preferably every two weeks (q2w).

The terms “biweekly dosing regimen,” “biweekly dosing”, “q2w” and “biweekly administration,” as used herein, refer to the time course of administering an anti-IL-36R antibody, or an antigen-binding portion thereof to a subject to achieve a therapeutic objective, e.g., throughout the course of treatment. The biweekly dosing regimen is not intended to include a weekly dosing regimen. Preferably, the substance is administered every 9-19 days, or 10-18 days, more preferably, every 11-17 days, or 12-16 days, even more preferably, every 13-15 days, and most preferably, every 14 days. In one embodiment, the biweekly dosing regimen is initiated in a subject at week 8 of treatment. In another embodiment, a maintenance dose is administered on a biweekly dosing regimen. In one embodiment, both the loading and maintenance doses are administered according to a biweekly dosing regimen. In one embodiment, biweekly dosing includes a dosing regimen where doses of an anti-IL-36R antibody, or an antigen-binding portion thereof are administered to a subject every other week consecutively for a given time period, e.g., 4 weeks, 8 weeks, 16, weeks, 24 weeks, 26 weeks, 32 weeks, 36 weeks, 42 weeks, 48 weeks, 52 weeks, 56 weeks, etc.

The terms “qwk,” “qw,” or “ew,” as used interchangeably herein, refer to a weekly dosing regimen, where anti-IL-36R antibody, or an antigen-binding portion thereof is administered to a subject once a week (or every week) to achieve a therapeutic objective, e.g., treating HS. A “weekly dosing regimen” as used herein, refers to the time course of administering a substance (e.g., an anti-IL-36R antibody) to a subject to achieve a therapeutic objective, e.g., throughout the course of treatment. Weekly administration is more frequent than biweekly, e.g., every 6-8 days, every 5-8 days, or every 7 days.

The term “intravenous infusion” refers to introduction of an agent into the vein of an animal or human patient over a period of time greater than approximately 15 minutes, generally between approximately 30 to 90 minutes.

The term “intravenous bolus” or “intravenous push” refers to drug administration into a vein of an animal or human such that the body receives the drug in approximately 15 minutes or less, generally 5 minutes or less.

The method of claim 11, further comprising subcutaneously administering a maintenance of at least 1200 mg to the subject comprising at least one dose of 300 mg, 600 mg, 900 mg, or 1200 mg of said anti-IL-36R antibody.

The term “subcutaneous administration” refers to introduction of an agent under the skin of an animal or human patient, preferable within a pocket between the skin and underlying tissue, by relatively slow, sustained delivery from a drug receptacle. Pinching or drawing the skin up and away from underlying tissue may create the pocket.

The term “subcutaneous infusion” refers to introduction of a drug under the skin of an animal or human patient, preferably within a pocket between the skin and underlying tissue, by relatively slow, sustained delivery from a drug receptacle for a period of time including, but not limited to, 30 minutes or less, or 90 minutes or less. Optionally, the infusion may be made by subcutaneous implantation of a drug delivery pump implanted under the skin of the animal or human patient, wherein the pump delivers a predetermined amount of drug for a predetermined period of time, such as 30 minutes, 90 minutes, or a time period spanning the length of the treatment regimen.

The term “subcutaneous bolus” refers to drug administration beneath the skin of an animal or human patient, where bolus drug delivery is less than approximately 15 minutes; in another aspect, less than 5 minutes, and in still another aspect, less than 60 seconds. In yet even another aspect, administration is within a pocket between the skin and underlying tissue, where the pocket may be created by pinching or drawing the skin up and away from underlying tissue.

Dosage regimens described herein may be adjusted (e.g., in individual patients) to provide the optimum desired response, e.g., maintaining remission of hidradenitis suppurativa, in consideration of the teachings herein.

It is to be noted that dosage values can vary with the type and severity of hidradenitis suppurativa. It is to be further understood that for any particular subject, specific dosage regimens may be adjusted over time according to the teachings of the specification and the individual need and the professional judgment of the person administering or supervising the administration of the compositions, and that dosage amounts and ranges set forth herein are exemplary only and are not intended to limit the scope or practice of the claimed invention.

A “pharmaceutical formulation” or “formulation” refers to the process but also the product of a process in which an active drug or agent is combined with chemical substances to produce a final medicinal or drug product, the final formulation therefore refers to medicinal products such as liquids, powders or compositions. Therefore, in one embodiment, a pharmaceutical formulation is a pharmaceutical composition.

A “pharmaceutical composition” refers in this context to a liquid or powder preparation which is in such form as to permit the biological activity of the active ingredient(s) to be unequivocally effective, and which contains no additional components which are significantly toxic to the subjects to which the composition would be administered. Such compositions are sterile. A “powder” refers to a freeze-dried or lyophilized or a spray-dried pharmaceutical composition for parenteral use. The powder is reconstituted or dissolved typically in water. Lyophilization is a low temperature dehydration process which involves freezing the product, lowering pressure, then removing the ice by sublimation. Freeze drying results in a high-quality product because of the low temperature used in processing. For a well-developed lyophilized formulation, the shape and appearance of the product is maintained over time and the quality of the rehydrated product is excellent. Spray drying is another method of producing a dry powder from a liquid or slurry by rapidly drying with a hot gas and with the goal of achieving a consistent particle size distribution.

As used herein “buffer” refers to a buffered solution that resists changes in pH by the action of its acid-base conjugate components. The “pH” herein refers to the acidity or basicity of the composition at room temperature. Standard methods to measure the pH of a composition are known to the skilled in the art. Typically, measuring pH consists of calibrating the instrument, placing the electrodes in a well-mixed sample, and then reading the pH directly from the pH meter. The exemplary buffers of the present invention include acetate, citrate, histidine, succinate, phosphate, and Tris.

As used herein, the term “tonicifying agent” or “tonicity agent” or “tonicifer” refers to substances providing an osmotic pressure equivalent to that of serum in the body including salts (e.g., sodium chloride, potassium chloride, magnesium chloride) or sugars (e.g., sucrose, trehalose, sorbitol, magnesium sulfate (MgSO₄), glycerol, mannitol or dextrose). In addition, sugars present in the solution act as a cryoprotectant for the protein which allows the drug substance to be frozen without damage. This permits shipment in the frozen form and long-term storage of the drug substance prior to the filling of drug product. The exemplary tonicifying agents of the present invention include sodium chloride, potassium chloride, magnesium chloride (salts) and/or sucrose, trehalose, sorbitol, magnesium sulfate (MgSO₄), glycerol, mannitol or dextrose (sugars).

As used herein, the term “stabilizer” or “stabilizing agent” refers to substances contributing to the stability of the active ingredient in a pharmaceutical formulation. The exemplary stabilizing agents of the present invention include arginine, histidine, glycine, cysteine, proline, methionine, lysine, or pharmaceutically acceptable salts thereof.

As used herein, the term “surfactant” refers to substances which tend to reduce the surface tension of a liquid in which they are dissolved. The exemplary surfactants of the present invention include poloxamer 188, polysorbate 20, polysorbate 40, polysorbate 60 or polysorbate 80.

In an embodiment relating to any of the above aspects, the anti-IL-36R antibody or an antigen binding fragment thereof (disclosed herein) is present in a stable pharmaceutical formulation (as described in co-pending U.S. application Ser. No. 16/809,606, filed Mar. 5, 2020, the entire content of which is hereby incorporated herein by reference in its entirety) for administration to a mammal or patient according to any one of the aspects of the present invention.

In one embodiment, the method of treatment according to any of the aspects described herein, includes administering to the mammal or patient a therapeutic amount of a stable pharmaceutical formulation comprising from about 20 mg/mL to about 150 mg/ml of an anti-IL-36R antibody (disclosed herein), about 20 mM to about 80 mM of a pharmaceutically acceptable buffer (e.g., acetate buffer), about 100 mM to about 250 mM of a pharmaceutically acceptable tonicifying agent (e.g., sucrose), about 0 mM to about 80 mM of a pharmaceutically acceptable stabilizing agent (e.g., arginine) or a pharmaceutically acceptable salt thereof, about 0 to about 150 mM of a pharmaceutically acceptable salt (e.g., sodium chloride), and a pharmaceutically acceptable surfactant (e.g., polysorbate 20) in an amount about 0 g/L to about 1.5 g/L, wherein the hidradenitis suppurativa (HS) in the patient is treated, prevented or ameliorated, or the skin disorder associated with HS in the patient is treated, or skin inflammation associated with HS in the patient is reduced or alleviated, or neutrophilic infiltrates in affected skin tissues in a subject suffering from HS in the patient is prevented and/or reduced, or the microbial colonization of the skin in a subject with HS is reduced, or susceptibility to a skin infection in subject suffering from HS is reduced. in the patient is achieved, or the complete resolution of HS symptoms in the patient is achieved, In a related embodiment, the stable pharmaceutical formulation is an aqueous pharmaceutical formulation. In a related embodiment, the pH of the aqueous pharmaceutical formulation is about 5 to about 7. In a related embodiment, the pharmaceutical formulation is for an intravenous administration to the mammal or patient. In a related embodiment, the pharmaceutical formulation is for a subcutaneous administration to the mammal or patient. In a related embodiment, the pharmaceutical formulation for an intravenous administration comprises an anti-IL-36R antibody in an amount of about 60 mg/mL, wherein one vial contains 450 mg. In a related embodiment, the pharmaceutical formulation for a subcutaneous administration comprises an anti-IL-36R antibody in an amount of about 150 mg/mL, wherein one pre-filled syringe contains 300 mg of antibody for subcutaneous injection.

### Treatment of Hidradenitis Suppurativa

The term “subject” for purposes of treatment refers to any animal classified as a mammal, including humans, domesticated and farm animals, and zoo, sports, or pet animals, such as dogs, horses, cats, cows, and the like. Preferably, the mammal is human.

Hidradenitis suppurativa (HS) is an inflammatory disease characterized by recurrent, painful abscesses and fistulous tracts. More specifically, HS is a skin disorder of the apocrine glands (sweat glands found on certain parts of the body) and hair follicles in which swollen, painful, inflamed lesions or lumps develop in the groin and sometimes under the arms and under the breasts. Hidradenitis suppurativa occurs when apocrine gland outlets become blocked by perspiration or are unable to drain normally because of incomplete gland development. Secretions trapped in the glands force perspiration and bacteria into surrounding tissue, causing subcutaneous induration, inflammation, and infection. Hidradenitis suppurativa is confined to areas of the body that contain apocrine glands. These areas are the axillae, areola of the nipple, groin, perineum, circumanal, and periumbilical regions.

Individuals with HS objectively have one of the lowest quality of life measures of any dermatologic disease. Lesions characteristically occur in the axillary, groin, infra-mammary, and/or anogenital regions of the body. HS lesions may progress to form sinus tracts and expansive abscesses. Sequelae include significant pain, scarring, and psychological distress. It has been shown that IL36 ligands are upregulated in the lesional skin of HS individuals and in circulation (Di Caprio et al., 2017; Hessam et al., 2018; Thomi et al., 2017).

While there is no animal model data for HS, expression and function of this pathway in various disease relevant cell types link it to skin inflammation. IL36R is expressed in epithelial cells (e.g., keratinocytes, intestinal epithelial cells), dermal fibroblasts, and immune cells (myeloid cells, B cells and T cells). Mice deficient of IL36R receptor had significantly reduced skin inflammation and keratinocyte proliferation compared to wild-type controls.

IL36R is a novel member of the IL1R family that forms a heterodimeric complex with the IL1R accessory protein (IL1RAcp) and IL1Rrp2 associated with epithelial mediated inflammation and barrier dysfunction. The heterodimeric IL36R system with stimulating (IL36α, IL36β, IL36Y) and inhibitory ligands (IL36Ra and IL38) shares a number of structural and functional similarities to other members of the IL1/ILR family, such as IL1, IL18 and IL33. All IL1 family members (IL1α, IL1β, IL18, IL36α, IL36β, IL36γ, and IL38) signal through a unique, cognate receptor protein which, upon ligand binding, recruits the common IL1RAP subunit and activates NFγB and MAP kinase pathways in receptor-positive cell types (Dinarello, 2011; Towne et al., 2004; Towne et al., 2011

Therefore, the present invention includes methods for treating a subject in need thereof, said method comprising administering a therapeutically effective amount of an anti-IL-36R antibody or an antigen binding fragment thereof to the subject. As used herein, the expression “a subject in need thereof” means a human or a non-human animal that exhibits one or more symptoms of a hidradenitis suppurative (HS), e.g., neutrophilic infiltration in affected skin tissues, pustules, etc., and/or who has been diagnosed with a HS condition.

In an embodiment, a subject suffering from a hidradenitis suppurativa may also have a skin infection selected from the group consisting of impetigo, cellulitis, infected dermatitis, eczema herpeticum, folliculitis, infected blister, mycosis, tinea versicolor, Staphylococcus aureus infection, and Streptococcus infection. A microbe that cause the infection includes, but is not limited to Staphylococcus aureus, Streptococcus spp., Pseudomonas aeruginosa, Bacteroides spp., Herpes simplex virus, coxsackievirus, molluscum contagiosum virus, vaccinia virus, Candida albicans, Microsporum spp., Trichophyton spp., Penicillium spp., Cladosporium spp., Alternaria spp., and Aspergillus spp.

The present invention provides methods to reduce microbial colonization of the skin in a subject with a hidradenitis suppurativa comprising administering a therapeutically effective amount of an anti-IL-36R antibody or an antigen binding fragment thereof to the subject. In certain embodiments, the invention provides for methods to reduce colonization of S. aureus on the skin of individuals with a neutrophilic dermatosis. In some embodiments, the microbial colonization is reduced by at least 10%, at least 15%, at least 20%, at least 25%, at least 30%, at least 35%, at least 40%, at least 45%, at least 50%, at least 55%, at least 60%, at least 65%, at least 70%, or at least 75% as compared to the baseline, upon administration of the anti-IL-36R antibody.

Microbial colonization may be measured with tests and procedures known in the art, e.g., by PCR, microbial culture, microscopy and staining or immunofluorescence. In certain embodiments, microbial colonization may be measured by the presence of microbial protein biomarkers known in the art, e.g., microbial toxin such as staph toxic shock syndrome toxin-1. Methods for detecting and/or quantifying such biomarkers are known in the art.

As used herein, the terms “treat”, “treating”, or the like, mean to alleviate symptoms, eliminate the causation of symptoms either on a temporary or permanent basis, or to prevent or slow the appearance of symptoms of the named disorder or condition. These terms are meant to include therapeutic as well as prophylactic, or suppressive measures for a disease or disorder leading to any clinically desirable or beneficial effect, including but not limited to alleviation or relief of one or more symptoms, regression, slowing or cessation of progression of the disease or disorder. Thus, for example, the term treatment includes the administration of an agent prior to or following the onset of a symptom of a disease or disorder thereby preventing or removing one or more signs of the disease or disorder. As another example, the term includes the administration of an agent after clinical manifestation of the disease to combat the symptoms of the disease. Further, administration of an agent after onset and after clinical symptoms have developed where administration affects clinical parameters of the disease or disorder, such as the degree of tissue injury, whether or not the treatment leads to amelioration of the disease, comprises “treatment” or “therapy” as used herein. Moreover, as long as the compositions of the invention either alone or in combination with another therapeutic agent alleviate or ameliorate at least one symptom of a disorder being treated as compared to that symptom in the absence of use of the humanized anti-IL-36R antibody composition, the result should be considered an effective treatment of the underlying disorder regardless of whether all the symptoms of the disorder are alleviated or not.

The term “therapeutically effective amount” is used to refer to an amount of an active agent that relieves or ameliorates one or more of the symptoms of the disorder being treated. In another aspect, the therapeutically effective amount refers to a target serum concentration that has been shown to be effective in, for example, slowing disease progression. Efficacy can be measured by methodology understood by those of skill in the art, for example by measures and methodology as described below where a proportion of individuals with a response to the administration of anti-IL36R is statistically significantly higher as compared to individuals on placebo for one or more of end points.

The term “prophylactically effective amount” is used to refer to an amount effective, at dosages and for periods of time necessary, to achieve the desired prophylactic result. Typically, a prophylactic dose is used in subjects prior to the onset of a HS outbreak and/or prior to the onset of symptoms of HS such as to prevent or inhibit the occurrence of an HS flare. In an embodiment, a subcutaneous dose as contemplated herein is a prophylactic dose that is used in a patient with acute HS, after the intravenous dose, to prevent a possible recurrence of the HS in the patient.

### Therapeutic Endpoints:

Embodiments of the invention provides a means for treating individuals suffering from moderate to severe chronic hidradenitis suppurativa.

In other embodiments, the invention provides improved methods of treatment, including methods of improving disease reduction in individuals having hidradenitis suppurativa and improvements in quality of life for the hidradenitis suppurativa individuals.

In other embodiments, the invention provides a method for treating certain subpopulations of individuals, including, for example, those who have failed prior therapy or have had as sub-therapeutic response, including, for example, a subject who has an inadequate response to or is intolerant to, or has a contraindication to, oral antibiotics. In certain embodiments, the invention is used to treat HS in a subject who was unresponsive or intolerant to oral antibiotics for treatment for their hidradenitis suppurativa, or a subject who has an inadequate response to or is intolerant to or has a contraindication to TNF-α inhibitors. In certain embodiments, the invention is used to treat HS in a subject who was unresponsive or intolerant to TNF-α for treatment for their hidradenitis suppurativa.

The methods and uses described herein provide a means of determining the efficacy of an anti-IL-36R antibody, or an antigen-binding portion thereof for treating hidradenitis suppurativa, and the use of such an anti-IL-36R antibody for treating hidradenitis suppurativa. Treatment of HS may be determined using any of the measures described herein, or any measure known to those in the art, for example:
(a) Percent change from baseline in dT count at Week 12
(b) Achievement of Hidradenitis Suppurativa Clinical Response (HiSCR) at Week 12.
(c) Absolute change from baseline in International Hidradenitis Suppurativa Severity Score System (IHS4) value at Week 12
(d) Absolute change from baseline in Hidradenitis suppurativa area and severity index (HASI) score at Week 12
(e) Achievement of at least 30% reduction from baseline in Numerical rating scale (NRS30) in Patient's Global Assessment of HS Pain at Week 12
(f) Achievement of Physician global assessment (PGA) score of 0 or 1 at Week 12
(g) Occurrence of complete elimination of dT at Week 12
(h) Occurrence of at least one flare (defined as at least 25% increase in AN count with a minimum increase of 2 relative to baseline up to Week 12.
(i) Absolute change from baseline in Dermatology life quality index (DLQI) Score at Week 12.
(j) Absolute change from baseline in Hidradenitis suppurativa quality of life (HiS-QOL) Total Score at Week 12.

In another embodiment related to any of the above embodiments or aspects first to seventh, the administration results in one or more of the following efficacy endpoints:
(a) Percent change from baseline in dT count at Weeks 8 and/or 16;
(b) Absolute change from baseline in IHS4 value at Week 8 and/or 16;
(c) Absolute change from baseline in Hidradenitis Suppurativa Area Severity Index (HASI) score at Week 16;
(d) Achievement of Hidradenitis Suppurativa Clinical Response (HiSCR50) at Week 16;
(e) Percent change from baseline in abscess count at Week 16;
(f) Achievement of at least a 50% reduction in dT count at Week 16 relative to baseline;\
(g) Achievement of at least a 50% reduction in abscess and dT (AdT) count at Week 16 relative to baseline;
(h) Achievement of at least a 50% reduction in ANdT count at Week 16 relative to baseline;
(i) Achievement of at least 30% reduction from baseline in Numerical rating Scale (NRS30) in Patient's Global Assessment of HS pain at Week 16;
(j) Percent change from baseline in dT count at each scheduled assessment;
(k) Achievement of at least 50% reduction from baseline in dT count at each scheduled assessment;
(l) Occurrence of complete elimination of dT at each scheduled assessment;
(m) Time to first occurrence of complete elimination of dT during the treatment period;
(n) Percent change from baseline in abscess count at each scheduled assessment;
(o) Achievement of at least 50% reduction from baseline in abscess count at each scheduled assessment;
(p) Occurrence of complete elimination of abscess at each scheduled assessment;
(q) Percent change from baseline in total AdT count at each scheduled assessment;
(r) Achievement of at least 50% reduction from baseline in AdT count at each scheduled assessment;
(s) Percent change from baseline in inflammatory nodule (N) count at each scheduled assessment;
(t) Achievement of at least 30% reduction from baseline in Numerical rating scale (NRS30) in Patient's Global Assessment of HS pain at each scheduled assessment;
(u) Absolute change from baseline in Hidradenitis Suppurativa Quality of Life (HiS-QoL) total score at each scheduled assessment;
(v) Absolute change from baseline in FACIT-Fatigue scale score at each scheduled assessment;
(w) Absolute change from baseline in Dermatology Life Quality Index (DLQI) score at each scheduled assessment;
(x) Absolute change from baseline in Patient Global Impression of Change (PGI-C) score over time.
(y) Absolute change from baseline in Patient Global Impression of Severity (PGI-S) score over time;
(z) Change from baseline in Hidradenitis Suppurativa odour and drainage scale (HODS) at each scheduled assessment;
(aa) Change from baseline in NRS Pruritus at each scheduled assessment;
(bb) Change from baseline in Hospital Anxiety and Depression Scale (HADS) at each scheduled assessment.
(cc) Percent change from baseline in total ANdT count at each scheduled assessment; or
(dd) Achievement of at least 50% reduction from baseline in ANdT count at each scheduled assessment.

In another embodiment related to above embodiments or aspects above, the proportion of individuals with a response to the administration is statistically significantly higher as compared to individuals on placebo for one or more of end points (a)-(j) and/or (a)-(dd).

In another embodiment related to above embodiments, the invention provides a method for decreasing the number of inflammatory lesions (AN count) or achievement of Hidradenitis suppurativa Clinical Responsiveness (HiSCR) in a subject having HS, said method comprising systemically administering an isolated human anti-IL36R. antibody, or an antigen binding portion thereof, to the subject, such that the AN count is decreased. Clinical Responsiveness according to the HiSCR scoring system is defined as at least a 50% reduction from baseline in the total AN count, with no increase in the abscess or draining fistula count.

In another embodiment related to above embodiments, the subject has no increase in an abscess count and/or no increase in a draining fistula or tunnel (dT) count following administration with the anti-IL36R antibody, or an antigen binding portion thereof.

In another embodiment related to above embodiments, the invention provides a method for decreasing the severity of HS according to the IHS4 value, which is the absolute change from baseline according to the International Hidradenitis Suppurativa Severity Score System (IHS4) for the assessment of HS severity. Determining IHS4 requires counting nodules, abscesses, and draining fistulae/sinus tracts. The absolute change from baseline or “IHS4” score assesses the HS severity and the resulting IHS4 score is arrived at by the number of nodules (multiplied by 1) plus the number of abscesses (multiplied by 2) plus the number of draining tunnels (multiplied by 4). A total score of 3 or less signifies mild, 4-10 signifies moderate and 11 or higher signifies severe disease.

In another embodiment related to above embodiments, the invention provides a method for improving (or reducing) the symptoms of HS as measured by the severity of HS symptoms according to the Hidradenitis Suppurativa Area and Severity Index (HASI) score of a subject having hidradenitis suppurativa from a high score (e.g., an HASI score of 3 or more in one or more regions) to a no or small impact score (e.g., an HS-PGA score of 0-2), comprising administering an IL-36R inhibitor, e.g., human IL-36R. antibody, or antigen-binding portion thereof, to the subject, such that the HASI score improves from the high score to the no or Small impact score. The HASI score is modelled after the Psoriasis Activity and Severity Index (PASI). Four classic signs of HS-related inflammation (erythema, induration, open ulcer, and draining tunnels) are included. Each variable in HASI is scored on a Likert scale (0-3) for each predetermined body region. For BSA assessment, the number of palms (one palm indicated 1% of the patient's BSA) involved for each body region (head, right axilla, left axilla, anterior chest, back, anterior bathing trunk, posterior bathing trunk, other) is assessed. This is converted to a percentage of that region. An area score was assigned to each region using the PASI approach (0=none, 1=1-9%, 2=10-29%, 3=30-49%, 4=50-69%, 5=70-89%, 6=90-100%). To calculate the regional HASI score, the sum of the 4 clinical variable scores is multiplied by the area score of each involved region. This value is then multiplied by the proportion of the BSA of that region, to give a regional HASI score. Regional HASI scores are added together to give the cumulative total HASI score (range 0-72). “HASI” combines the assessment of the severity of lesions and the area affected into a single score in the range 0 (no disease) to 72 (severe disease).

In another embodiment related to above embodiments, the invention provides a method of improving (i.e., reducing) a Hidradenitis Suppurativa-Physicians Global Assessment (HS-PGA) score. the invention also provides a method of decreasing an HS PGA score of a subject having hidradenitis suppurativa by at least about 2 grades, comprising administering an IL36R inhibitor, e.g., human IL36R. antibody, or antigen-binding fragment thereof. The HS-PGA document is the physician's assessment of the patient's HS at a given time-point. It scores patient disease severity as either clear, minimal, mild, moderate to severe, or very severe, on the basis of abscesses, draining fistulae, inflammatory nodule and non-inflammatory nodule (Kimball A B, Kerdel F, Adams D, et al. Adalimumab for the treatment of moderate to severe hidradenitis suppurativa: a parallel randomized trial. Ann Intern Med 2012; 157:846-855). The “HS-PGA score” ranges from 0 to 5, where 0 (clear: no abscesses, draining tunnels, inflammatory nodules or noninflammatory nodules), 1 (minimal: no abscesses, draining tunnels or inflammatory nodules and the presence of noninflammatory nodules), 2 (mild: no abscesses or draining tunnels and 1-4 inflammatory nodules, or 1 abscess or draining tunnel and no inflammatory nodules), 3 (moderate: no abscesses or draining tunnels and ≥5 inflammatory nodules, or 1 abscess or draining tunnel and ≥1inflammatory nodule, or 2-5 abscesses or draining tunnels and <10 inflammatory nodules), 4 (severe: 2-5 abscesses or draining tunnels and ≥10 inflammatory nodules), 5 (very severe: >5 abscesses or draining tunnels).

In another embodiment related to above embodiments, the invention provides a method of decreasing an HS-PGA score of a subject having hidradenitis suppurativa from a high score (e.g., a score of 3 or more) to a no or small impact score (e.g., a score of 0-2), comprising administering a human IL-36R antibody to the subject, such that the HS-PGA score is decreased from the high score to the no or small impact score.

In another embodiment related to above embodiments, the invention provides a method of decreasing an HS-PGA score of a subject having hidradenitis suppurativa by at least about 2 grades, comprising administering a human IL-36R antibody to the subject, such that the HS-PGA score is decreased by at least about 2 grades.

In another embodiment related to above embodiments, the Numerical rating scale (NRS30) in Patient's Global Assessment of Hidradenitis suppurativa (HS) pain may also be used as an index for measuring efficacy of an anti-IL36R antibody, or an antigen-binding portion thereof in a patient or patient population having hidradenitis suppurativa, where mean statistically significant (as compared to placebo) improvement within a population of treated individuals in their NRS30 Pain score that is at least 30% indicates that the IL-36R antibody is effective for treating hidradenitis suppurativa. The HS Pain NRS is an endpoint for the assessment of HS-related pain severity for clinical trials with individuals with HS. Response was given by an 11-point scale ranging from 0 (no HS pain) to 10 (HS pain as bad as one can imagine). In related embodiment, the invention provides a method for determining whether a human IL-36R. antibody is effective for treating hidradenitis suppurativa based on improvement in NRS30 Pain score.

In another embodiment related to above embodiments, the invention provides a method of improving a Dermatology Life Quality Index (DLQI) score of a subject having hidradenitis suppurativa from a high score (i.e., the quality of life is impaired) to a low score (i.e., the quality of life not impacted or low impact) comprising administering a human IL-36R antibody to the subject, such that the DLQI score is decreased from the high score to the low score. The DLQI is a patient-administered, ten-question, quality of life questionnaire that covers six domains including symptoms and feelings, daily activities, leisure, work and school, personal relationships and treatment (Finlay A Y, Khan G K. Dermatology Life Quality Index (DLQI)—a simple practical measure for routine clinical use. Joint Ann Mtg of the British Association of Dermatologists and the Canadian Dermatology Association, Oxford, 6-10 Jul. 1993. Clin Exp Dermatol 1994; 19:210-216). The DLQI has a one week recall period. Response categories include “not relevant” (score of 0), “not at all” (score of 0), “a little” (score of 1), “a lot” (score of 2) and “very much” (score of 3). Question 7 is a “yes”/“no” question where “yes” is scored as 3. The DLQI total score is calculated by summing the scores of each question resulting in a range of 0 to 30. The higher the score, the more the quality of life is impaired.

In another embodiment related to above embodiments, the invention provides a method of improving a Hidradenitis Suppurativa Quality of Life (HiS-QOL) score of a subject having hidradenitis suppurativa from a high score (i.e., the quality of life is highly impacted) to a low score (low impact on the quality of life) comprising administering a human IL-36R antibody to the subject, such that the HiS-Qol score is decreased from the high score to the low score. The HiS-Qol survey is a patient-administered, 17-item instrument to measure HS-specific quality of life in clinical trials. The 17-item HiS-Qol included four symptom items, eight activity-adaptation items and five psychosocial items. The item scores are summed to create a total ranging from 0 to 68, with higher scores indicating more severe impact on quality of life.

In another embodiment related to above embodiments, the invention provides a method of improving symptoms of HS or reducing pruritus severity related to HS as scored according to the Pruritus Numerical Rating Scale (NRS Pruritus) comprising administering a human IL-36R antibody to the subject, such that the score is decreased from the high baseline score to a low score. The HS Pruritus NRS is an endpoint for the assessment of HS-related pruritus severity for clinical trials with individuals with HS. The Patient Global Assessment of HS Pruritus assess the worst HS pruritus. Ratings range from 0 (no itch) to 10 (HS worst imaginable itch). It is a unidimensional measure of pruritus intensity and can be administered daily with minimal trial participant burden. Recall period is 24 h and response is given by an 11-point scale ranging from 0 (“no itch”) to 10 (“worst imaginable itch”). Trial participants are asked to rate the intensity of their itch using this scale.

In another embodiment related to above embodiments, the invention provides a method of improving symptoms of HS or reducing the incidence of drainage and odour resulting from clinical symptoms of HS, according to the Hidradenitis Suppurativa Odour and Drainage scale (HODS), comprising administering a human IL-36R antibody to the subject, such that the score is decreased from the high baseline score to a low score. The HODS is an 8-item scale developed to assess the HS-related drainage and odour in individuals. It covers two domains: drainage (5 items) and odour (3 items). The response options range from 1-5. Higher scores depict worse outcome of the concept being assessed.

In another embodiment related to above embodiments, the invention provides a method of reducing and or ameliorating anxiety and/or depression of individuals with HS according to the Hospital Anxiety and Depression Scale (HADS), comprising administering a human IL-36R antibody to the subject, such that the score is decreased from the high baseline score to a low score. The HADS is an instrument for screening anxiety and depression in non-psychiatric populations. The HADS consists of 14 items, 7 each for anxiety and depression symptoms; possible scores range from 0 to 21 for each subscale.

In another embodiment related to above, the invention provides a method of improving the impact of HS on a patient's work productivity, and/or overall activity according to the Work Productivity and Activity Impairment Questionnaire for HS (WPAI-HS) comprising administering a human IL-36R antibody to the subject, such that the score is decreased from the high baseline score (high impact) to a low score (low impact). The WPAI-HS is 6-item instrument to assess the effect of hidradenitis suppurativa on ability to work and perform normal daily activities.

In another embodiment related to above embodiments, the invention provides a method of decreasing the impact of HS on a patient's quality of life and/or improving a patient's overall quality of life as evaluated by EuroQol 5 dimensions 5-level (EQ-5D-5L), comprising administering a human IL-36R antibody to the subject, wherein an improvement is a change from an high impact score to a low impact score. The descriptive system comprises five dimensions: mobility, self-care, usual activities, pain/discomfort, and anxiety/depression. Each dimension has 5 levels: no problems, slight problems, moderate problems, severe problems, and extreme problems. The EQ VAS records the patient's self-rated health on a vertical visual analogue scale, where the endpoints are labelled ‘The best health you can imagine’ and ‘The worst health you can imagine’.

In another embodiment related to above embodiments, proportion of individuals with a response to the administration of anti-IL36R is statistically significantly higher as compared to individuals on placebo for one or more of end points.

In one aspect, the present invention relates to a method of treating and/or preventing the recurrence of HS symptoms in a patient treated with one or more intravenous dose(s) of the anti-IL-36R antibody according to any of aspects or the above embodiments, said method including administering to the patient a therapeutically effective amount of the anti-IL-36R antibody in one or more subcutaneous doses.

In one aspect, the present invention relates to a method of achieving a 50% reduction from baseline in the total AN count, with no increase in the abscess or draining fistula count in a patient treated with one or more dose(s) of the anti-IL-36R antibody according to any of aspects of the above embodiments, said method including administering to the patient an effective amount of the anti-IL-36R antibody in one or more intravenous and/or subcutaneous doses.

In one aspect, the present invention relates to a method of achieving improvement of one or more HS symptoms in a patient treated with one or more intravenous dose(s) followed by one or more subcutaneous dose(s) of the anti-IL-36R antibody according to any of aspects or the above embodiments; wherein the HS symptoms comprise inflammatory lesions, abscesses, draining fistulae/sinus tracts or tunnels (dT), HS associated inflammation (erythema, induration, open ulcers), HS associated infection, and/or HS associated pain.

In one embodiment related to any of aspects above or the related embodiment(s), at least 10%, 20%, 30%, 40%, 50%, 60%, 70% or 80% of the individuals show significant clinical improvement as measured by percent change from baseline in total AN count at Week 8, 12, 16, 20, 24, 36, 48, 52, 60 or 72 of the treatment.

In one embodiment related to any of aspects above or the related embodiment(s), at least 10%, 20%, 30%, 40%, 50%, 60%, 70% or 80% of the individuals show significant clinical improvement in the occurrence of HS flare (the incidence of flare defined as at least 25% increase in AN count with a minimum increase of 2 relative to baseline total AN count at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show significant clinical improvement as measured by a change in percent change from baseline in total dT count at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show complete elimination of dT at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals achieve clinical responsiveness as measured by Hidradenitis Suppurativa Clinical Response score HiSCR defined as at least a 50% reduction from baseline in the total AN count, with no increase in the abscess or draining fistula count at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show significant clinical improvement as measured by an absolute change from baseline in International Hidradenitis Suppurativa Severity Score system (IHS4) at Week 8, 12, 16, 20, 24, 36, 48, 52, 60 or 72 of the treatment.

In one embodiment related to any of aspects above or the related embodiment(s), at least 10%, 20%, 30%, 40%, 50%, 60%, 70% or 80% of the individuals show significant clinical improvement as measured by an absolute change from baseline in Hidradenitis Suppurativa Area and Severity Index (HASI) at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show 30% reduction from baseline in Numerical rating scale (NRS30) in Patient's Global Assessment of HS Pain at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show a score of 0 or 1 measured by the Physician Global Assessment (PGA) at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show clinical improvement as measured by absolute change from baseline in the Dermatology life quality index (DLQI) Score at Week 8, 12, 16, 20, 24, 36, 48, 52, 60 or 72 of the treatment.

In one embodiment related to any of aspects above or the related embodiment(s) at least 10%, 20%, 30%, 40%, 50%, 60%, 70% or 80% of the individuals show clinical improvement as measured by absolute change from baseline in the Hidradenitis suppurative quality of life (HiS-QoL) Total Score at Week 8, 12, 16, 20, 24, 36, 48, 52, 60 or 72 of the treatment.

In a related embodiment, proportion of individuals with a response to the administration is statistically significantly higher as compared to individuals on placebo for any of the end points recited.

### Pharmaceutical Doses and Administration

Anti-IL-36R antibodies of the present invention are typically administered to a patient as a pharmaceutical composition in which the antagonist is admixed with a pharmaceutically acceptable carrier or excipient, see, e. g., Remington's Pharmaceutical Sciences and US. Pharmacopeia: National Formulary, Mack Publishing Company, Easton, Pa. (1984). The pharmaceutical composition may be formulated in any manner suitable for the intended route of administration. Examples of pharmaceutical formulations include lyophilized powders, slurries, aqueous solutions, suspensions and sustained release formulations (see, e.g., Hardman et al. (2001), Goodman and Gilman's The Pharmacological Basis of Therapeutics, McGraw-Hill, New York, N. Y.; Gennaro (2000) Remington: The Science and Practice of Pharmacy, Lippincott, Williams, and Wilkins, New York, N. Y.; Avis et al. (eds.) (1993) Pharmaceutical Dosage Forms: Parenteral Medications, Marcel Dekker, NY; Lieberman et al. (eds.) (1990) Pharmaceutical Dosage Forms: Tablets, Marcel Dekker, NY; Lieberman et al. (eds.) (1990) Pharmaceutical Dosage Forms: Disperse Systems, Marcel Dekker, NY; Weiner and Kotkoskie (2000) Excipient Toxicity and Safety, Marcel Dekker, Inc., New York, N. Y.). Suitable routes of administration include intravenous injection (including intraarterial injection) and subcutaneous injection.

In one embodiment, the invention provides a method for treating a subject having hidradenitis suppurativa (HS), the method comprising administering an isolated human anti IL-36R antibody, or an antigen binding portion thereof, to the subject according to a multiple variable dose regimen, such that HS is treated, wherein the multiple variable dose regimen comprises administering at least one or more intravenous doses. In a related embodiment, each of the one or more intravenous dose(s) includes at least 450, 600, 900, 1200, or 1800 mg of said anti-IL-36R antibody, wherein the total loading dose of said anti-IL-36R is at least 1200 mg, 1350 mg, 1600 mg, 1800 mg, 2400 mg, 2700 mg, 3600 mg, 4800 mg, 5400 mg, or 7200 mg.

In another embodiment, the anti-IL-36R antibody is administered in one intravenous dose. In another embodiment related to any of the above aspects, the anti-IL-36R antibody is administered in at least one intravenous dose of 450 mg. In another embodiment related to any of the above aspects, the anti-IL-36R antibody is administered in one intravenous dose of 600 mg. In another embodiment related to any of the above aspects, the anti-IL-36R antibody is administered in one intravenous dose of 900 mg. In another embodiment related to any of the above aspects, the anti-IL-36R antibody is administered in one intravenous dose of 1200 mg. In another embodiment related to any of the above aspects, the anti-IL-36R antibody is administered in one intravenous dose of 1800 mg.

In another embodiment 1, 2, 3, or 4 intravenous dose(s) is/are administered. In another embodiment related to any of the above aspects, 2, 3, or 4 intravenous doses are administered at 1, 2, 4, 6-, 8-, 10- or 12-weeks intervals.

In another embodiment, 1, 2, 3, or 4 intravenous dose(s) is/are administered, and at least 1, 2, 3, 4, or 5 subcutaneous dose(s) is/are administered to the subject at 1, 2, 4, 6, 8, 10 or 12, 16-week intervals after the last intravenous dose.

In one embodiment, the invention provides a method for treating a subject having hidradenitis suppurativa (HS), the method comprising administering an isolated human anti IL-36R antibody, or an antigen binding portion thereof, to the subject according to a multiple variable dose regimen, such that HS is treated, wherein the multiple variable dose regimen comprises administering at least one or more intravenous doses. In a related embodiment, each of the one or more intravenous dose(s) includes at least of 450 mg, 900 mg, 1200 mg, or 1800 mg of said anti-IL-36R antibody, wherein the total loading dose is at least 1200 mg, 1350 mg, 1600 mg, 1800 mg, 2400 mg, 2700 mg, 3600 mg, 4800 mg, 5400 mg, or 7200 mg. In a related embodiment, the anti-IL-36R antibody is administered in a loading dose of at least 1800 mg comprising one or more intravenous doses of 450 mg, 900 mg, 1200 mg, or 1800 mg of said anti-IL-36R antibody. In a related embodiment, the anti-IL-36R antibody is administered in four intravenous doses of 450 mg administered; two, three, or four intravenous doses of 900 mg; two, three, or four intravenous doses of 1200 mg; or two, three, or four intravenous doses of 1800 mg. In a related embodiment 2, 3, or 4 intravenous doses are administered at 1, 2, 3, to 4-week intervals.

In one embodiment related to any of the above aspects and embodiments, the anti-IL-36R antibody is administered in one or more intravenous dose(s), wherein the total loading dose is at least 1200 mg, 2400 mg, or 3600 mg. In a preferred embodiment, three intravenous doses of 1200 mg are delivered at weeks 0, 1, and 2, for a total loading dose of 3600 mg.

In one embodiment related to any of the above aspects and embodiments, the anti-IL-36R antibody is administered in one or more intravenous dose(s), wherein the total loading dose is 3600 mg, followed by five subcutaneous doses of 1200 mg every two weeks at weeks 4, 6, 8, 10, and 12.

In one embodiment related to any of the above aspects and embodiments, the anti-IL-36R antibody is administered in one or more intravenous dose(s), wherein the total loading dose is at least 1800 mg, 3600 mg, or 7200 mg. In non-limiting, exemplary embodiments, four intravenous doses of 450 mg are delivered at weeks 0, 1, and 2, 3 for a total loading dose of 1800 mg; four intravenous doses of 900 mg are delivered at weeks 0, 1, and 2, 3 for a total loading dose of 3600 mg; or four intravenous doses of 1800 mg are delivered at weeks 0, 1, and 2, 3 for a total loading dose of 7200 mg.

In one embodiment related to any of the above aspects and embodiments, the anti-IL-36R antibody is administered in one or more intravenous dose(s), wherein the total loading dose is at least 1800 mg, 3600 mg, or 7200 mg, followed by at least one subcutaneous dose of 300 mg, 600 mg, or 1200 mg every week at weeks 4, 5, 6, and 7. In non-limiting, exemplary embodiments, four subcutaneous doses of 300 mg are delivered once weekly at weeks 4, 5, 6, and 7 for a total maintenance dose of 1200 mg; four subcutaneous doses of 600 mg are delivered once weekly at weeks 4, 5, 6, and 7 for a total maintenance dose of 2400 mg; or four subcutaneous doses of 1200 mg are delivered once weekly at weeks 4, 5, 6, and 7 for a total maintenance dose of 3600 mg. In a related embodiment, the at least one subcutaneous dose is administered after the final intravenous dose.

In one embodiment related to maintenance dose above, in a non-limiting exemplary regimen, the anti-IL-36R antibody is administered subcutaneously at a dose of 600 mg, or 1200 mg biweekly following the last subcutaneous dose comprising the maintenance dose.

Representative examples of doses and dose regimens according to the present invention are disclosed in Table 12 and Tables 22 and 23.

### Antibodies of the Present Invention

The anti-IL36R antibodies of the present invention are disclosed in U.S. Pat. No. 9,023,995 or WO2013/074569, the entire content of each of which is incorporated herein by reference.

The term “antibody,” as used herein, includes immunoglobulin molecules comprising four polypeptide chains, two heavy (H) chains and two light (L) chains inter-connected by disulfide bonds, as well as multimers thereof (e.g., IgM). In a typical antibody, each heavy chain comprises a heavy chain variable region (abbreviated herein as HCVR or V_{H}) and a heavy chain constant region. The heavy chain constant region comprises three domains, C_{H}1, C_{H}2 and C_{H}3. Each light chain comprises a light chain variable region (abbreviated herein as LCVR or V_{L}) and a light chain constant region. The light chain constant region comprises one domain (CL1). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of the anti-IL-36R antibody (or antigen-binding portion thereof) may be identical to the human germline sequences or may be naturally or artificially modified. An amino acid consensus sequence may be defined based on a side-by-side analysis of two or more CDRs.

The term “antibody,” as used herein, also includes antigen-binding fragments of full antibody molecules. The terms “antigen-binding portion” of an antibody, “antigen-binding fragment” of an antibody, and the like, as used herein, include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)2 fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the V_{H} and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

The antibodies used in the methods of the present invention may be human antibodies. The term “human antibody,” as used herein, is intended to include antibodies having variable and constant regions derived from human germline immunoglobulin sequences. The human antibodies of the invention may nonetheless include amino acid residues not encoded by human germline immunoglobulin sequences (e.g., mutations introduced by random or site-specific mutagenesis in vitro or by somatic mutation in vivo), for example in the CDRs and in particular CDR3. However, the term “human antibody,” as used herein, is not intended to include antibodies in which CDR sequences derived from the germline of another mammalian species, such as a mouse, have been grafted onto human framework sequences.

The antibodies used in the methods of the present invention may be recombinant human antibodies. The term “recombinant human antibody,” as used herein, is intended to include all human antibodies that are prepared, expressed, created or isolated by recombinant means, such as antibodies expressed using a recombinant expression vector transfected into a host cell (described further below), antibodies isolated from a recombinant, combinatorial human antibody library (described further below), antibodies isolated from an animal (e.g., a mouse) that is transgenic for human immunoglobulin genes (see e.g., Taylor et al. (1992) Nucl. Acids Res. 20:6287-6295) or antibodies prepared, expressed, created or isolated by any other means that involves splicing of human immunoglobulin gene sequences to other DNA sequences. Such recombinant human antibodies have variable and constant regions derived from human germline immunoglobulin sequences. In certain embodiments, however, such recombinant human antibodies are subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

According to certain embodiments, the antibodies used in the methods of the present invention specifically bind IL-36R. The term “specifically binds,” or the like, means that an antibody or antigen-binding fragment thereof forms a complex with an antigen that is relatively stable under physiologic conditions. Methods for determining whether an antibody specifically binds to an antigen are well known in the art and include, for example, equilibrium dialysis, surface plasmon resonance, and the like. For example, an antibody that “specifically binds” IL-36R, as used in the context of the present invention, includes antibodies that bind IL-36R or portion thereof with a K_{D} of less than about 1000 nM, less than about 500 nM, less than about 300 nM, less than about 200 nM, less than about 100 nM, less than about 90 nM, less than about 80 nM, less than about 70 nM, less than about 60 nM, less than about 50 nM, less than about 40 nM, less than about 30 nM, less than about 20 nM, less than about 10 nM, less than about 5 nM, less than about 4 nM, less than about 3 nM, less than about 2 nM, less than about 1 nM or less than about 0.5 nM, as measured in a surface plasmon resonance assay. An isolated antibody that specifically binds human IL-36R may, however, have cross-reactivity to other antigens, such as IL-36R molecules from other (non-human) species.

In certain exemplary embodiments related to any aspects of the present invention, the anti-IL-36R antibody or antigen-binding fragment thereof that can be used in the context of the methods of the present invention includes: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 or 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
i. a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
ii. a) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iii. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
iv. a) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
v. a) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3) or
vi. a) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3); or
vii. a) a light chain variable region comprising the amino acid sequence of SEQ ID NO: 26 (L-CDR1); the amino acid sequence of SEQ ID NO: 104 (L-CDR2); the amino acid sequence of SEQ ID NO: 44 (L-CDR3); and b) a heavy chain variable region comprising the amino acid sequence of SEQ ID NO: 141 (H-CDR1); the amino acid sequence of SEQ ID NO: 62, 108, 109, 110, 111 or 142 (H-CDR2); the amino acid sequence of SEQ ID NO: 72 (H-CDR3).

According to certain embodiments, the anti-IL-36R antibody or antigen-binding fragment thereof comprises:
i. a light chain variable region comprising the amino acid sequence of SEQ ID NO: 77; and a heavy chain variable region comprising the amino acid sequence of SEQ ID NO: 87; or
ii. a light chain variable region comprising the amino acid sequence of SEQ ID NO: 77; and a heavy chain variable region comprising the amino acid sequence of SEQ ID NO: 88; or
iii. a light chain variable region comprising the amino acid sequence of SEQ ID NO: 77; and a heavy chain variable region comprising the amino acid sequence of SEQ ID NO: 89; or
iv. a light chain variable region comprising the amino acid sequence of SEQ ID NO: 80; and a heavy chain variable region comprising the amino acid sequence of SEQ ID NO: 87; or
v. a light chain variable region comprising the amino acid sequence of SEQ ID NO: 80; and a heavy chain variable region comprising the amino acid sequence of SEQ ID NO: 88; or
vi. a light chain variable region comprising the amino acid sequence of SEQ ID NO: 80; and a heavy chain variable region comprising the amino acid sequence of SEQ ID NO: 89; or
vii. a light chain variable region comprising the amino acid sequence of SEQ ID NO: 85; and a heavy chain variable region comprising the amino acid sequence of SEQ ID NO: 100; or
viii. a light chain variable region comprising the amino acid sequence of SEQ ID NO: 85; and a heavy chain variable region comprising the amino acid sequence of SEQ ID NO:101; or
ix. a light chain variable region comprising the amino acid sequence of SEQ ID NO: 86; and a heavy chain variable region comprising the amino acid sequence of SEQ ID NO: 100; or
x. a light chain variable region comprising the amino acid sequence of SEQ ID NO: 86; and a heavy chain variable region comprising the amino acid sequence of SEQ ID NO:101.

According to certain embodiments, the anti-IL-36R antibody or antigen-binding fragment thereof comprises:
i. a light chain comprising the amino acid sequence of SEQ ID NO: 115; and a heavy chain comprising the amino acid sequence of SEQ ID NO: 125; or
ii. a light chain comprising the amino acid sequence of SEQ ID NO: 115; and a heavy chain comprising the amino acid sequence of SEQ ID NO: 126; or
iii. a light chain comprising the amino acid sequence of SEQ ID NO: 115; and a heavy chain comprising the amino acid sequence of SEQ ID NO: 127; or
iv. a light chain comprising the amino acid sequence of SEQ ID NO: 118; and a heavy chain comprising the amino acid sequence of SEQ ID NO: 125; or
v. a light chain comprising the amino acid sequence of SEQ ID NO: 118; and a heavy chain comprising the amino acid sequence of SEQ ID NO: 126; or
vi. a light chain comprising the amino acid sequence of SEQ ID NO: 118; and a heavy chain comprising the amino acid sequence of SEQ ID NO: 127; or
vii. a light chain comprising the amino acid sequence of SEQ ID NO: 123; and a heavy chain comprising the amino acid sequence of SEQ ID NO: 138; or
viii. a light chain comprising the amino acid sequence of SEQ ID NO: 123; and a heavy chain comprising the amino acid sequence of SEQ ID NO: 139; or
ix. a light chain comprising the amino acid sequence of SEQ ID NO: 124; and a heavy chain comprising the amino acid sequence of SEQ ID NO: 138.

In one aspect, described and disclosed herein are anti-IL-36R antibodies, in particular humanized anti-IL-36R antibodies, and compositions and articles of manufacture comprising one or more anti-IL-36R antibody, in particular one or more humanized anti-IL-36R antibody of the present invention. Also described are binding agents that include an antigen-binding fragment of an anti-IL-36 antibody, in particular a humanized anti-IL-36R antibody.

In one aspect, described and disclosed herein is an anti-IL-36R antibody that is spesolimab.

### Mode of Action

An anti-IL-36R antibody of the present invention is a humanized antagonistic monoclonal IgG1 antibody that blocks human IL36R signaling. Binding of an anti-IL-36R antibody of the present invention to IL36R is anticipated to prevent the subsequent activation of IL36R by cognate ligands (IL36 α, β and γ) and downstream activation of pro-inflammatory and pro-fibrotic pathways with the aim to reduce epithelial cell/fibroblast/immune cell-mediated inflammation and interrupt the inflammatory response that drives pathogenic cytokine production.

IL-36R is also known as IL-1RL2 and IL-1Rrp2. It has been reported that agonistic IL-36 ligands (α, β, or γ) initiate the signaling cascade by engaging the IL-36 receptor which then forms a heterodimer with the IL-1 receptor accessory protein (IL-1RAcP). IL-36 antagonist ligands (IL-36RA/IL1F5, IL-38/ILF10) inhibit the signaling cascade.

### Articles of Manufacture

In another aspect, an article of manufacture containing materials useful for the treatment of the disorders described above is included. The article of manufacture comprises a container and a label. Suitable containers include, for example, bottles, vials, syringes, and test tubes (e.g., to hold the unit dose forms). The containers may be formed from a variety of materials such as glass or plastic. The container holds a composition that is effective for treating the condition and may have a sterile access port. For example, the container may be an intravenous solution bag or a vial having a stopper pierceable by a hypodermic injection needle. The active agent in the composition is the humanized anti-IL-36R antibody. The label on or associated with the container indicates that the composition is used for treating the condition of choice. The article of manufacture may further comprise a second container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use.

The term “package insert” is used to refer to instructions customarily included in commercial packages of therapeutic products, which contain information about the indications, usage, administration, contraindications and/or warnings concerning the use of such therapeutic products.

The invention is further described in the following examples, which are not intended to limit the scope of the invention.

### EXAMPLES

The following examples are intended to further illustrate certain preferred embodiments of the disclosure and are not limiting in nature. Those skilled in the art will recognize or be able to ascertain numerous equivalents to the specific substances and procedures described herein.

### Example 1: Phase IIa Clinical Trial: Treating Individuals with Hidradenitis Suppurativa

### Objectives:

Currently, Adalimumab (TNFi) is the only approved biologic for HS, with the response rate of 42% to 59% versus placebo response of 26% to 28%, with a schedule of weekly s.c. dosing. Even available treatment options for controlling of HS, complete resolution of symptoms, and prevention of reoccurrence of HS have their limitations, with regard to evidence of efficacy and safety). In addition, compared to other inflammatory diseases, such as psoriasis, long-term safety concerns are pertinent in the treatment of HS, where the dosing regimen of biologics is typically more intensive. Considering the above, a more efficacious and safer molecule with better dosing regimens is a substantial need in individuals with HS.

IL-36 alpha, beta, and gamma mRNA expression are upregulated in lesional skin in HS individuals with a decrease in IL-36RN expression (Hessam S, Sand M, Gambichler T, et al. Interleukin-36 in hidradenitis suppurativa: evidence for a distinctive proinflammatory role and a key factor in the development of an inflammatory loop. Br J Dermatol 2018; 178:761-767). Internal in-situ hybridization staining of HS lesions also confirms the increased expression of ligands. IL-36 is thought to be a central upstream mediator of an inflammatory loop which further activates the keratinocytes, amplifying the secretion of chemokines that lead to infiltration of immune cells to the skin. Infiltrated dendritic cells (DCs) and monocytes can be activated by IL-36 (internal data and published) to potentiate the antigen presenting cell function of DCs and also lead to secretion of chemokines and cytokines that can recruit more immune cells including neutrophils. IL-36 is a potent activator of neutrophil infiltration and in the context of HS, neutrophils are shown to undergo netosis leading to secretion of AMPs and type I Interferons (Byrd A S, Carmona-Rivera C, O'Neil L J, et al. Neutrophil extracellular traps, B cells, and type I interferons contribute to immune dysregulation in hidradenitis suppurativa. Sci Transl Med 2019; 11). Based on the role of IL-36 driving TH17 and TH1 responses and from animal models of atopic dermatitis (AtD), the hypothesis was that IL-36 might be a key driver of TH17 responses in skin of HS patients. Given that, spesolimab might be beneficial in treatment of HS.

In this example, an anti-IL36R antibody of the present invention is used to treat individuals with hidradenitis suppurativa. The purpose of this study is to find out whether a medicine called spesolimab helps people with moderate to severe hidradenitis suppurativa.

The primary objective of this trial was to estimate the effect of spesolimab compared to placebo for the mean percent change from baseline in total abscess and inflammatory nodule (AN) count at Week 12 in individuals with moderate to severe HS. Secondary objectives were the evaluation of efficacy of spesolimab on secondary endpoints versus placebo. The comparisons of interest for each efficacy objective were those of all treated individuals but excluding the effects of rescue therapy. Further objectives of this trial were the evaluation of further measures of efficacy, the PK, anti-drug antibody (ADA) of spesolimab and exploration of specific biomarkers in individuals with HS.

HS is characterized by recurrent, painful abscesses and fistulous tracts. Individuals with HS objectively have one of the lowest quality of life measures of any dermatologic disease. Lesions characteristically occur in the axillary, groin, infra-mammary, or anogenital regions of the body. HS lesions may progress to form sinus tracts and expansive abscesses. Sequelae include significant pain, scarring, and psychological distress. The average age of onset is during the early 20s (Zouboulis C C, Marmol V del, Mrowietz U, et al. Hidradenitis suppurativa/acne inversa: criteria for diagnosis, severity assessment, classification and disease evaluation. Dermatology (Basel) 2015; 231(2):184-190). The global prevalence of HS is reported between 0.0003% and 4.1%. Underdiagnosis or improper diagnosis is common. Overall, HS prevalence varies significantly based on study methodology; however, the disease appears to be more common than was previously considered (Saunte D M, Boer J, Stratigos A, et al. Diagnostic delay in hidradenitis suppurativa is a global problem. Br J Dermatol 2015; 173(6):1546-1549).

Some of the most burdensome HS symptoms from patient perspective are pain, drainage and explosive openings, itch, skin tightness (scarring), odour, fatigue, and flu-like symptoms. Individuals reported to be unsatisfied with the level of control offered by currently available treatment options; unmet needs from the patient perspective include the need for new medical treatments with favorable efficacy and tolerability profiles. In qualitative evidence, the most important treatment goals from the patient perspective were pain, drainage (including explosive openings), and fatigue.

In the Phase IIa trial 1368.52 Participants are put into 2 groups; the placebo group, or those taking spesolimab. Participants get spesolimab or placebo as an infusion into a vein every week for the first 3 weeks. Afterwards they get spesolimab or placebo as injections under the skin every 2 weeks.

The primary objective of the Phase IIa trial was to estimate the effect of spesolimab compared to placebo for the mean percent change from baseline in total abscess and inflammatory nodule (AN) count at Week 12 in individuals with moderate to severe HS. Secondary objectives were the evaluation of efficacy of spesolimab on secondary endpoints versus placebo. The comparisons of interest for each efficacy objective were those of all treated individuals but excluding the effects of rescue therapy.

Further objectives of this trial were the evaluation of further measures of efficacy, the PK, anti-drug antibody (ADA) of spesolimab and exploration of specific biomarkers in individuals with HS.

### Overall Design:

A parallel group, randomized, double-blind, and placebo-controlled trial was considered the most appropriate to demonstrate PoC. The placebo control group was required to compare both efficacy and safety of spesolimab in individuals with moderate-to-severe HS who were biologic naïve or have failed previous TNFi treatments. And a 2:1 randomization schema could help reducing the number of individuals assigned to the placebo arm. Additionally, if eligible, individuals were to be invited to roll over to an OLE trial where individuals were to take active drug.

The treatment duration of 12 weeks with spesolimab was selected to evaluate sustained efficacy over flares in individuals with HS.

The loading dose of three i.v. administrations followed by the maintenance s.c. biweekly dosing was selected to optimize the exposure of spesolimab to evaluate its efficacy in HS. The intravenous loading dose was selected to maximize the treatment response, the duration of response and to reach steady state earlier. A loading dose was also considered to allow for an earlier onset of response. This was followed by maintenance dose given subcutaneously so as to sustain the response.

The Phase IIa trial 1368.52 was an international, multi-center, double-blind, placebo-controlled trial assessing the efficacy and safety of spesolimab in individuals with moderate to severe HS. A total of 52 individuals (spesolimab: 36 individuals; placebo: 16 individuals). After screening, individuals, who met all eligibility criteria, were randomized in a 2:1 ratio to either active group or placebo group. The randomization was stratified for TNFi naive population versus TNFi-failure population. Approximately 33 individuals from TNFi naive population and 12 individuals from TNFi-failure population were planned to be randomized, respectively. Primary failure of TNF treatment was defined as lack of efficacy after at least 3 months of treatment with an agent blocking TNF-alpha, and secondary failure was defined as loss of clinical efficacy in a patient after initial response to an agent blocking TNF-alpha.

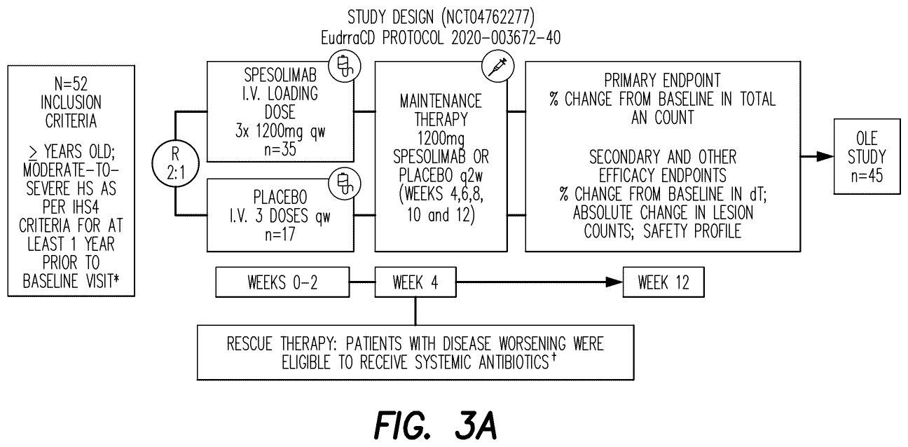

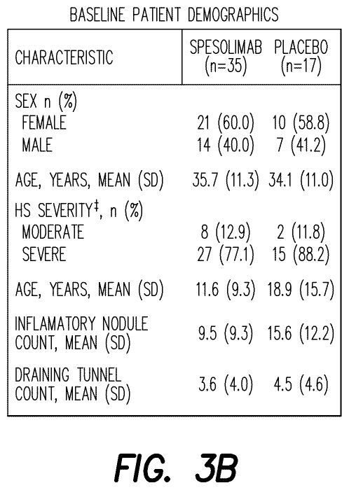

Once randomized, individuals started a treatment period of 12 weeks (see FIG. 3.A) with a loading dose of spesolimab 3600 mg i.v. (1200 mg i.v. q1w) or matching placebo, followed by maintenance s.c. dosing of 1200 mg spesolimab q2w or matching placebo.

For the ongoing open-label, long term extension trial (1368.67), an interim analysis was conducted in individuals with HS who had completed treatment in trial 1368.52. Individuals from the placebo arm of the 1368.52 trial are given an initial 1200 mg i.v. loading dose of spesolimab plus s.c. placebo, followed by 600 mg spesolimab s.c. q2w. Individuals from the active arm of the 1368.52 trial are given an initial i.v. placebo plus 600 mg spesolimab s.c., followed by 600 mg spesolimab s.c. q2w.

### Inclusion Criteria:

Individuals were to be enrolled (screened) into the trial at Visit 1, and treatment was to be initiated at Visit 2, if they met the following criteria:
Male or female adult individuals, 18 years of age or older
Signed and dated written informed consent in accordance with International Council on Harmonization (ICH) Good Clinical Practice (GCP) and local legislation prior to the start of any screening procedures.
Moderate to severe Hidradenitis suppurativa (HS), based on International Hidradenitis Suppurativa Severity Score System (IHS4) criteria, for at least 1 year prior to the baseline visit, as determined by the investigator through participant interview and/or review of the medical history. (If IHS4 scoring is not available, equivalent scoring based on scoring systems as HS-PGA or Hurley are acceptable based on documented investigator assessment)
HS lesions in at least 2 distinct anatomic area (right/left axillary, inguinal, inframammary, perineal)
Biologic naive or TNF inhibitor (TNFi)-failure for HS
Inadequate response to an adequate course of appropriate oral antibiotics for treatment of HS in the last 1 year, as per investigator discretion. This is not applicable for TNFi-failure individuals.
Total abscess and inflammatory nodule (AN) count of greater than or equal to 5.
Total draining fistula count of less than or equal to 20. Further inclusion criteria apply.

### Exclusion Criteria:

Individuals were not to be screened or treated if any of the following criteria applied:
Presence of active skin lesions other than HS that interfere with the assessment of HS.
Use of restricted medications as below:
Topical corticosteroids over HS lesions within 1 week of Visit 2
Systemic antibiotics within 4 weeks of visit 2
Systemic non-biologic immunomodulatory and/or immunosuppressive agents use for HS within 4 weeks (or 5 half-lives, whichever is longer) of visit 2.
Biologic agents use within 12 weeks or 5 half-lives, whichever is longer, prior to visit 2.
Opioid analgesics within 2 weeks of visit 2
Live virus vaccine within 6 weeks of visit 2
Prior exposure to any immunosuppressive biologic other than TNFi for HS
Prior exposure to Interleukin 36 Receptor (IL-36R) inhibitors including spesolimab.
Treatment with any investigational device or investigational drug of chemical or biologic nature within a minimum of 30 days or 5 half-lives of the drug, whichever is longer, prior to visit 2.
Women who are pregnant, nursing, or who plan to become pregnant while in the trial. Women who stop nursing before the study drug administration do not need to be excluded from participating.
History of allergy/hypersensitivity to the systemically administered trial medication agent or its excipients.
Patient with a transplanted organ (with exception of a corneal transplant>12 weeks prior to screening) or who have ever received stem cell therapy (e.g., Remestemcel-L) Further exclusion criteria apply.

### Relevant Trial Activities:

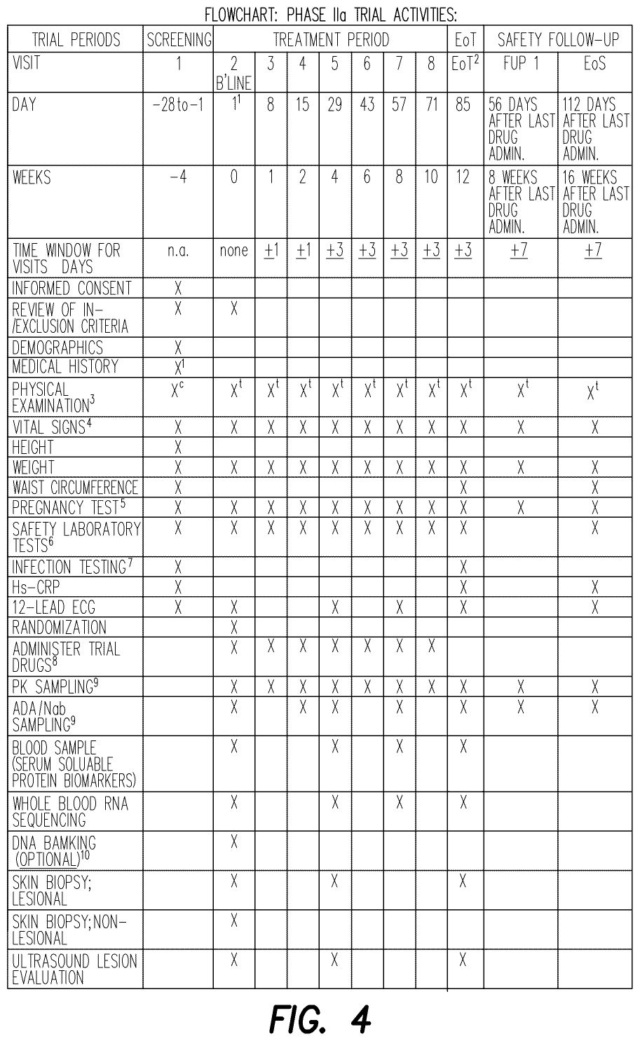

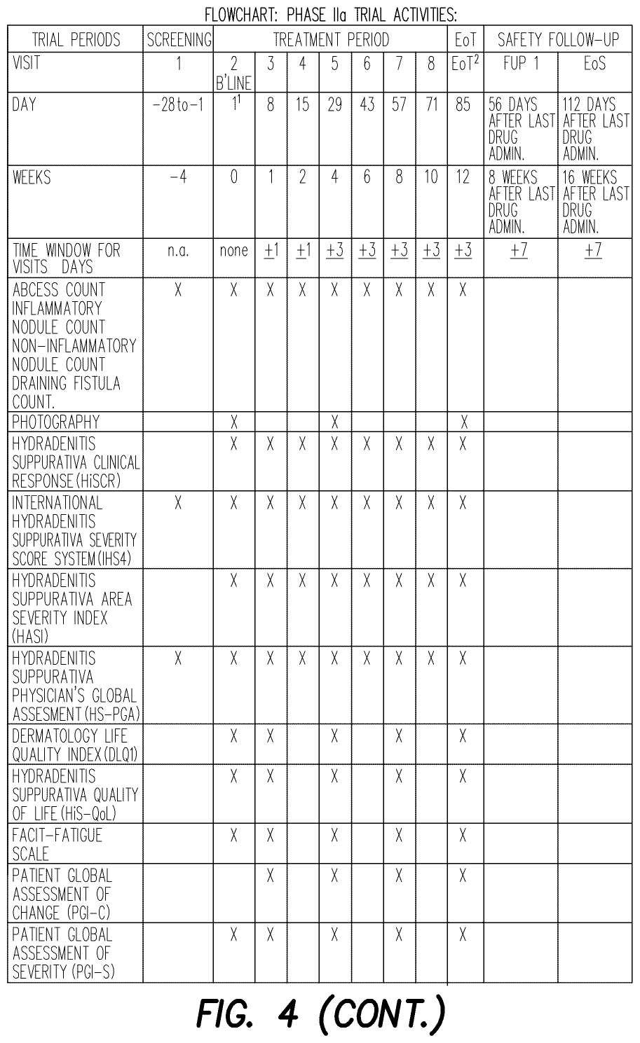

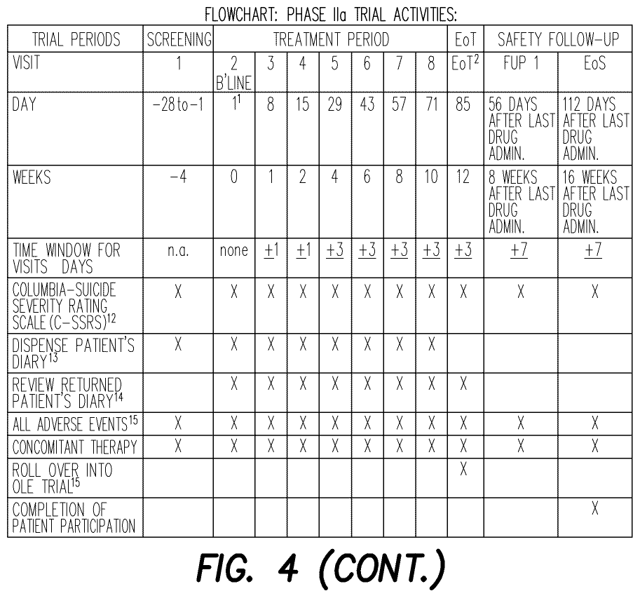

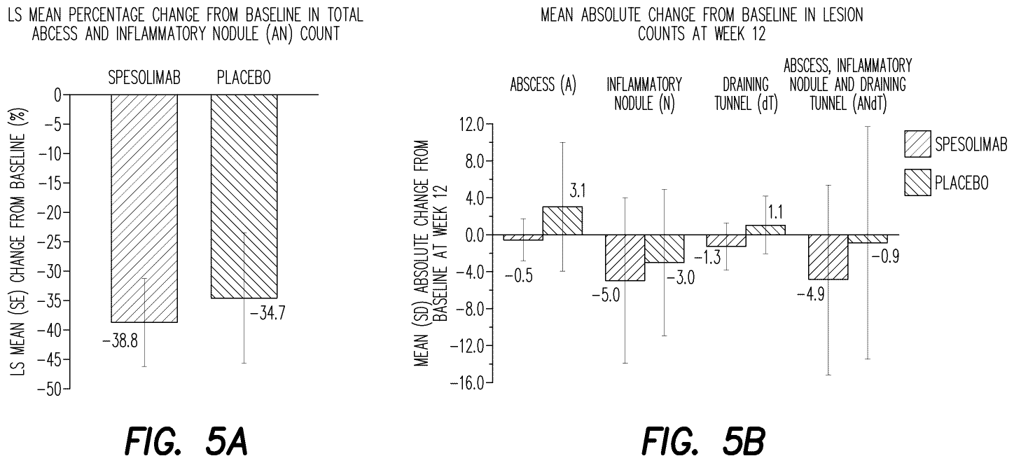

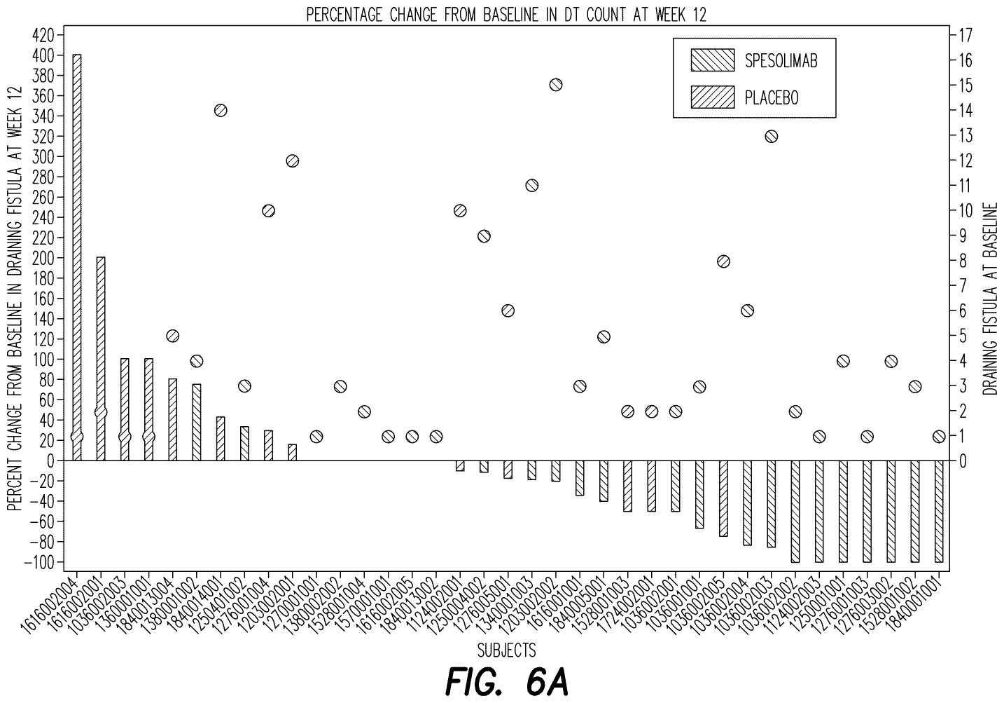

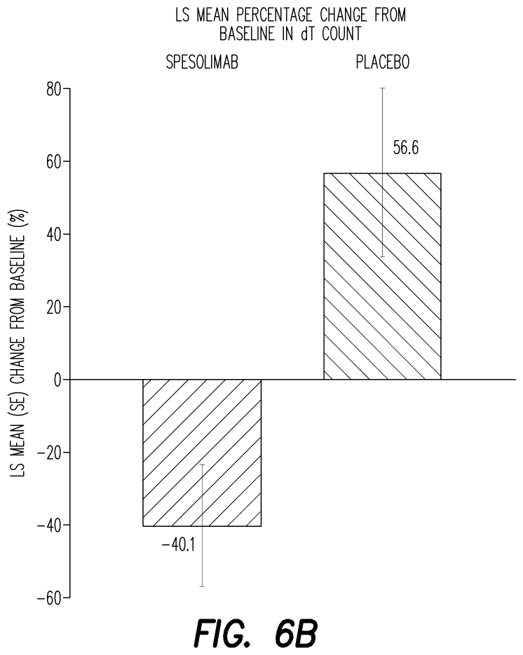

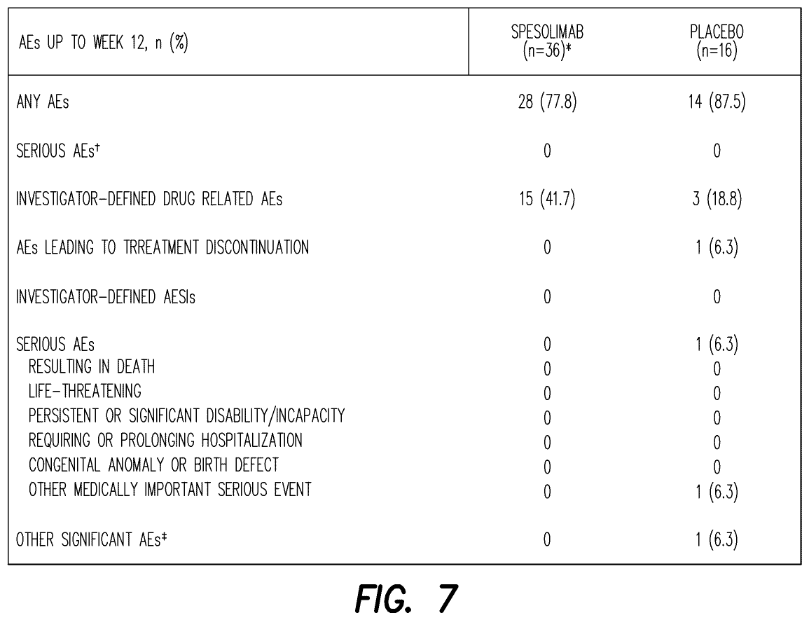

An overview of all relevant trial activities are shown in the Flowchart (FIG. 4). “Study Week” in the Flowchart represents the end of each week (e.g., end of Week 1=D8, end of Week 2=D15, end of Week 3=D22, end of Week 4=D29, etc.). Relevant activities are further described below as indicated as footnotes in the flowchart.

Day of Randomization/Day of first intake of randomized medication. If needed, some Visit 2 assessments may have been conducted 2 days before or after the actual visit date after consultation with the sponsor (FN1). Individuals who discontinued trial treatment prematurely should have undergone the EoT visit as soon as possible. All individuals not entering the OLE trial were expected to complete the EoT visit, a FUP1 visit 8 weeks after their last trial drug intake and a final EoS visit 16 weeks after their last trial drug intake. For individuals entering the OLE trial at Week 12, the EoT visit was to be their EoS visit in trial 1368-0052 (FN2 and see also FN 8 and FN16).

X^{c} are timepoints a complete physical examination is indicated. X^{t} indicates when a targeted physical examination is indicated (FN3). Measurements of vital signs preceded blood sampling to avoid the impact of blood sampling on the vital measurements at all dosing visits. Additional assessments of vital signs should have been performed at 10 minutes post-dose for s.c administrations and 5 minutes and 1 hour post dose for i.v. administrations (FN4).

For women of childbearing potential only a serum pregnancy test was performed at screening. Urine pregnancy tests were to be performed at all other visits indicated in the Flowchart (FIG. 4) (FN5).

At visits with study drug administration, the blood sampling for the safety laboratory should have been done prior to the study drug administration. It was preferred, but individuals did not have to be fasted for the blood sampling for the safety laboratory. If needed, safety lab samples for Visit 2 were allowed to be drawn within 48 hours before the actual visit date (FN 6).

Infection Testing was performed at screening and at EoT visit (FN 7).

Individuals who terminated study drug early should have done the EoT visit as soon as possible. These individuals were to do FUP1 and EoS visits, 8 and 16 weeks after last study drug administration, respectively (FN 8).

At study visits with study drug administration, pre-dose PK/ADA/NAb samples were to be obtained approximately within 1 hour prior to start of i.v. infusion or s.c. injection (FN9). Deoxyribonucleic Acid (DNA) banking sample was optional. This sampling was only possible if the patient agreed by signing a separate informed consent (FN10). At selected sites, where ultrasound was available, it was required to do ultrasound lesion evaluation and to guide biopsies. At sites without ultrasound capabilities, biopsies were to be done without ultrasound guidance (FN 11).

At Screening, the C-SSRS baseline/screening scale was to be completed. At all subsequent visits the “Since last visit” scale was to be completed. (FN 12).

At Visits 4, 5, 6, 7, and 8, two-weekly diaries and a buffer of one additional week to capture surplus time window of visit were to be dispensed to individuals. Patient Diary was to be used to capture daily NRS score, analgesic use, and other interventions to manage pain. (FN 13). Patient's Diaries returned by individuals had to be reviewed while the patient was in the consultation, so that any information was allowed to be clarified in an interview with the patient, if needed (FN 14).

Local tolerability at the administration site of spesolimab was assessed by the investigator during the study drug administration visit and at the time of AE assessment by questioning retrospectively since the last visit. Any observed local tolerability reaction, e.g., “swelling”, “induration”, “heat”, “redness” should have been reported as adverse event (FN 15).

All individuals completing Week 12 of the study may have been offered to enter the OLE study 1368-0067. These individuals were not to be requested to complete follow-up period and were to have their End of study visit at EoT visit, being coincident with the first visit in 1368-0067 (FN 16).

### Demographics and Relevant Baseline Efficacy Parameters:

In both treatment groups, more female (overall: 59.6%) than male (overall: 40.4%) individuals were randomized, and most of individuals were White (overall: 67.3%), followed by Asian (overall: 11.5%) and Not Stated (overall: 11.5%). The mean (SD) age was 35.2 (11.1) years and comparable in both treatment groups. The mean (SD) body weight was 93.87 (23.18) kg; minimum 53.5 kg, maximum 151.0 kg. The mean BMI (SD) were 32.28 (7.51) kg/m2 and over 30 kg/m2 in both treatment groups, which was characteristic and as expected in this patient population with HS. Overall, 73.1% of individuals (n=38) were TNFi-naïve population, and 26.9% of individuals (n=14) were TNFi-failure population.

All individuals met the target inclusion criteria of moderate to severe HS. At baseline, overall, 80.8% of individuals (n=42) had severe HS, and 19.2% of individuals (n=10) had moderate HS, as per IHS4. A marked imbalance in the mean (SD) number of inflammatory nodules was observed across treatment groups at baseline: spesolimab 9.5 (9.3), placebo 15.6 (12.2), while the mean number of abscesses and the mean number of dT were comparable across treatment groups. Almost half of individuals (n=24, 46.2%) had the total number of AN count.

TABLE 4
Demographic Data:

| | | | | | | Overall | |
|---|---|---|---|---|---|---|---|
| | | Placebo | | Spesolimab | | Total | |
| Number of patients | | 17 | (100.0) | 35 | (100.0) | 52 | (100.0) |
| (N, %) | | | | | | | |
| Sex (N, %) | Male | 7 | (41.2) | 14 | (40.0) | 21 | (40.4) |
| | Female | 10 | (58.8) | 21 | (60.0) | 31 | (59.6) |
| Race (N, %) | Asian | 2 | (11.8) | 4 | (11.4) | 6 | (11.5) |
| | Black or African | 2 | (11.8) | 2 | (5.7) | 4 | (7.7) |
| | American | | | | | | |
| | Native Hawaiian or | 0 | | 1 | (2.9) | 1 | (1.9) |
| | other | | | | | | |
| | Pacific Islander | | | | | | |
| | White | 12 | (70.6) | 23 | (65.7) | 35 | (67.3) |
| | Not stated | 1 | (5.9) | 5 | (14.3) | 6 | (11.5) |
| Ethnicity (N, %) | Hispanic or Latino | 1 | (5.9) | 1 | (2.9) | 2 | (3.8) |
| | Not Hispanic or | 15 | (88.2) | 29 | (82.9) | 44 | (84.6) |
| | Latino | | | | | | |
| | Not stated | 1 | (5.9) | 5 | (14.3) | 6 | (11.5) |
| Age [years] | Mean (SD) | 34.1 | (11.0) | 35.7 | (11.3) | 35.2 | (11.1) |
| | Median (min, max) | 35.0, | (20, 60) | 33.0 | (20, 61) | 33.5 | (20, 61) |
| Weight [kg] | Mean (SD) | 86.89 | (16.69) | 97.27 | (25.27) | 93.87 | (23.18) |
| | Median (min, max) | 88.50 | (61.0, 111.8) | 93.40 | (53.5, 151.0) | 92.50 | (53.5, 151.0) |
| Weight categories | ≤90 kg | 9 | (52.9) | 14 | (40.0) | 23 | (44.2) |
| (N, %) | >90 kg | 8 | (47.1) | 21 | (60.0) | 29 | (55.8) |
| Body mass index | Mean (SD) | 30.37 | (5.63) | 33.21 | (8.18) | 32.28 | (7.51) |
| [kg/m²] | Median (min, max) | 29.47 | (23.4, 39.1) | 32.85 | (22.0, 54.1) | 31.75 | (22.0, 54.1) |
| BMI categories (N, %) | <30 kg/m² | 9 | (52.9) | 15 | (42.9) | 24 | (46.2) |
| | ≥30 kg/m² | 8 | (47.1) | 20 | (57.1) | 28 | (53.8) |
| Stratification Factor | TNFi-failure | 4 | (23.5) | 10 | (28.6) | 14 | (26.9) |
| (N, %) | populations | | | | | | |
| | TNFi-naïve | 13 | (76.5) | 25 | (71.4) | 38 | (73.1) |
| | population | | | | | | |
| Smoking status (N, %) | Current | 9 | (52.9) | 14 | (40.0) | 23 | (44.2) |
| | Former | 1 | (5.9) | 6 | (17.1) | 7 | (13.5) |
| | Never | 7 | (41.2) | 15 | (42.9) | 22 | (42.3) |
| Alcohol status (N, %) | Current | 10 | (58.8) | 19 | (54.3) | 29 | (55.8) |
| | Former | 0 | | 5 | (14.3) | 5 | (9.6) |
| | Never | 7 | (41.2) | 11 | (31.4) | 18 | (34.6) |
| Nicotine status (N, %) | Current | 10 | (58.8) | 16 | (45.7) | 26 | (50.0) |
| | Former | 2 | (11.8) | 6 | (17.1) | 8 | (15.4) |
| | Never | 5 | (29.4) | 13 | (37.1) | 18 | (34.6) |
| Cannabis status (N, %) | Current | 1 | (5.9) | 2 | (5.7) | 3 | (5.8) |
| | Former | 1 | (5.9) | 5 | (14.3) | 6 | (11.5) |
| | Never | 15 | (88.2) | 28 | (80.0) | 43 | (82.7) |
| Vaping status (N, %) | Current | 0 | | 5 | (14.3) | 5 | (9.6) |
| | Former | 1 | (5.9) | 0 | | 1 | (1.9) |
| | Never | 16 | (94.1) | 30 | (85.7) | 46 | (88.5) |

If patients' information were not allowed to collect per local law, then it was categorized as Not stated.

### Primary Outcome Measures:

The primary efficacy endpoint in this trial was the percent change from baseline in total abscess and inflammatory nodule (AN) count at Week 12.

### Secondary Outcome Measures:

a. Percent change from baseline in draining fistula count at week 12 [Time Frame: baseline, week 12]
b. Achievement of Hidradenitis Suppurativa Clinical Response (HiSCR) at week 12 [Time Frame: week 12]. The HiSCR is one of the most well-known and widely used outcome assessment in clinical trials. HiSCR is defined as at least a 50% reduction from baseline in the total AN count, with no increase in the abscess or draining fistula count (Kimball A B, Sobell J M, Zouboulis C C, et al. HiSCR (Hidradenitis Suppurativa Clinical Response): a novel clinical endpoint to evaluate therapeutic outcomes in patients with hidradenitis suppurativa from the placebo-controlled portion of a phase 2 adalimumab study. J Eur Acad Dermatol Venereol 2016; 30:989-994). HiSCR was to be evaluated at the time-points mentioned in Table 6 (Also see Trial Activity Flowchart, FIG. 4. Achievement of clinical response or “HiSCR” is defined as at least a 50% reduction in the total AN count with no increase in abscess count and no increase in draining fistula count relative to baseline.
c. Absolute change from baseline in International Hidradenitis Suppurativa Severity Score System (IHS4) value at week 12 [Time Frame: baseline, week 12]. IHS4 is a valid, clinical scoring system for dynamic assessment of HS severity. Determining IHS4 requires counting nodules, abscesses, and draining fistulae/sinus tracts making it straightforward to apply in clinical trials (Zouboulis C C, Tzellos T, Kyrgidis A, et al, European Hidradenitis Suppurativa Foundation Investigator Group. Development and validation of the International Hidradenitis Suppurativa Severity Score System (IHS4), a novel dynamic scoring system to assess HS severity. Br J Dermatol 2017; 177:1401-1409). IHS4 was to be evaluated at the time-points mentioned in Table 6 (See also Trial Activity Flowchart, FIG. 4). The absolute change from baseline or “IHS4” score assesses the HS severity and the resulting IHS4 score is arrived at by the number of nodules (multiplied by 1) plus the number of abscesses (multiplied by 2) plus the number of draining tunnels (multiplied by 4). A total score of 3 or less signifies mild, 4-10 signifies moderate and 11 or higher signifies severe disease.
d. Absolute change from baseline in Hidradenitis Suppurativa Area and Severity Index (HASI) score at week12 [Time Frame: baseline, week 12]. HASI is modelled after the Psoriasis Activity and Severity Index (PASI). Four classic signs of HS-related inflammation (erythema, induration, open ulcer, and draining tunnels) are included. Each variable in HASI is scored on a Likert scale (0-3) for each predetermined body region. For BSA assessment, the number of palms (one palm indicated 1% of the patient's BSA) involved for each body region (head, right axilla, left axilla, anterior chest, back, anterior bathing trunk, posterior bathing trunk, other) is assessed. This is converted to a percentage of that region. An area score was assigned to each region using the PASI approach (0=none, 1=1-9%, 2=10-29%, 3=30-49%, 4=50-69%, 5=70-89%, 6=90-100%). To calculate the regional HASI score, the sum of the 4 clinical variable scores is multiplied by the area score of each involved region. This value is then multiplied by the proportion of the BSA of that region, to give a regional HASI score. Regional HASI scores are added together to give the cumulative total HASI score (range 0-72). HASI was to be evaluated at the time-points mentioned in Table 6 (See also Trial Activity Flowchart, FIG. 4). “HASI” combines the assessment of the severity of lesions and the area affected into a single score in the range 0 (no disease) to 72 (severe disease).
e. Achievement of Hidradenitis Suppurativa Physician Global Assessment (HS-PGA) score of 0 or 1 at week12 [Time Frame: week 1]. The HS-PGA document is the physician's assessment of the patient's HS at a given time-point. It scores patient disease severity as either clear, minimal, mild, moderate to severe, or very severe, on the basis of abscesses, draining fistulae, inflammatory nodule and non-inflammatory nodule (Kimball A B, Kerdel F, Adams D, et al. Adalimumab for the treatment of moderate to severe hidradenitis suppurativa: a parallel randomized trial. Ann Intern Med 2012; 157:846-855). HS-PGA was to be evaluated at the time-points mentioned in Table 6 (See also, Trial Activity Flowchart, FIG. 4). The “HS-PGA score” ranges from 0 to 5, where 0 (clear: no abscesses, draining tunnels, inflammatory nodules or noninflammatory nodules), 1 (minimal: no abscesses, draining tunnels or inflammatory nodules and the presence of noninflammatory nodules), 2 (mild: no abscesses or draining tunnels and 1-4 inflammatory nodules, or 1 abscess or draining tunnel and no inflammatory nodules), 3 (moderate: no abscesses or draining tunnels and ≥5 inflammatory nodules, or 1 abscess or draining tunnel and ≥1inflammatory nodule, or 2-5 abscesses or draining tunnels and <10 inflammatory nodules), 4 (severe: 2-5 abscesses or draining tunnels and ≥10 inflammatory nodules), 5 (very severe: >5 abscesses or draining tunnels).
f. Achievement of at least 30% reduction from baseline in Numerical rating scale (NRS30) in Patient's Global Assessment of Hidradenitis suppurativa (HS) Pain at week 12 [Time Frame: baseline, week 12]. The HS Pain NRS is an endpoint for the assessment of HS-related pain severity for clinical trials with individuals with HS. As it is a unidimensional measure of pain intensity and can be administered daily with minimal patient burden. Recall period was 24 h, and response was given by an 11-point scale ranging from 0 (no HS pain) to 10 (HS pain as bad as one can imagine). The Pain NRS is completed on a daily diary by subjects from Screening through Week 12.
g. Occurrence of complete elimination of draining fistulas at Week 12 [Time Frame: week 12]. Occurrence of at least one flare (defined as at least 25% increase in AN count with a minimum increase of 2 relative to baseline) up to Week 12 [Time Frame: up to week 12]
h. Absolute change from baseline in Dermatology Life Quality Index (DLQI) score at week 12 [Time Frame: baseline, week 12]. The DLQI is a patient-administered, ten-question, quality of life questionnaire that covers six domains including symptoms and feelings, daily activities, leisure, work and school, personal relationships and treatment (Table 5). The DLQI has a one week recall period. Response categories include “not relevant” (score of 0), “not at all” (score of 0), “a little” (score of 1), “a lot” (score of 2) and “very much” (score of 3). Question 7 is a “yes”/“no” question where “yes” is scored as 3. The DLQI total score was calculated by summing the scores of each question resulting in a range of 0 to 30 where 0-1=no effect on patient's life, 2-5=small effect, 6-10=moderate effect, 11-20=very large effect, and 21-30=extremely large effect on patient's life. The higher the score, the more the quality of life is impaired. If one question was left unanswered, this was to be scored 0, and the total score was to be summed and expressed as usual out of a maximum of 30; for analysis by sub-score only, the corresponding sub-score was to be considered to be missing. If 2 or more questions were left unanswered (missing), DLQI total score was to be treated as missing. A 4-point change from baseline is considered a clinically important difference.

TABLE 5
The DLQI was to be analyzed under six headings as follows:

| Domain | Question Number | Score |
|---|---|---|
| Symptoms and feelings | Question 1 and 2 | Maximum 6 |
| Daily activities | Questions 3 and 4 | Maximum 6 |
| Leisure | Questions 5 and 6 | Maximum 6 |
| Work and School | Question 7 | Maximum 3 |
| Personal Relationships | Questions 8 and 9 | Maximum 6 |
| Treatment | Question 10 | Maximum 3 |

Finlay A Y, Khan G K. Dermatology Life Quality Index (DLQI)—a simple practical measure for routine clinical use. Joint Ann Mtg of the British Association of Dermatologists and the Canadian Dermatology Association, Oxford, 6-10 Jul. 1993. Clin Exp Dermatol 1994; 19:210-216.
i. Absolute change from baseline in Hidradenitis Suppurativa Quality of Life (HIS-QoL) Total score at week12 [Time Frame: baseline, week 12]. “HiS-QoL” is a patient-administered, 17-item instrument to measure HS-specific quality of life in clinical trials. The 17-item HiS-Qol included four symptom items, eight activity-adaptation items and five psychosocial items. The item scores are summed to create a total ranging from 0 to 68, with higher scores indicating more severe impact on quality of life.
j-Functional Assessment of Chronic Illness Therapy (FACIT)-Fatigue The FACIT-Fatigue is a 13-item questionnaire (Webster K, Cella D, Yost K. The Functional Assessment of Chronic Illness Therapy (FACIT) Measurement System: properties, applications, and interpretation. Health Qual Life Outcomes 2003; 1:79; Yellen S B, Cella D F, Webster K, et al. Measuring fatigue and other anemia-related symptoms with the Functional Assessment of Cancer Therapy (FACT) measurement system. J Pain Symptom Manage 1997; 13(2):63-74; Cella D, Yount S, Sorensen M, et al. Validation of the Functional Assessment of Chronic Illness Therapy Fatigue Scale relative to other instrumentation in individuals with rheumatoid arthritis. J Rheumatol 2005; 32(5):811-819) that assesses self-reported fatigue and its impact upon daily activities and function. Answers are based on a 5-point Likert scale. Responses of “not at all”, “a little”, “somewhat”, “quite a bit”, and “very much” are available for each question, and correspond to scores of 0, 1, 2, 3, and 4, respectively (total score range: 0-52). A minimal clinically important difference (MCID) of 3-4 points in change score has been reported (Cella D, et al. 2005). The recall period for items is 7 days.
k. Patient Global Impression of Change (PGI-C) The PGI-C is a 1-item tool assessing the change of HS start of taking the study medication by 5-point Likert-type scale. The tool was required for anchoring of other instruments and endpoints.
l. Patient Global Impression of Severity (PGI-S) The PGI-S is a 1-item tool assessing the severity of HS over the last week by 4-point Likert-type Scale. The tool was required for anchoring of other instruments and endpoints.
m. The occurrence of Treatment Emergent Adverse Events (TEAEs) [Time Frame: up to week 16]. The intensity of the adverse event was to be assessed by the investigator and graded according to RCTC Version 2.0 developed by the Outcome Measures in Rheumatology (OMERACT) organization (Woodworth T, Furst D E, Alten R, et al. Standardizing assessment and reporting of adverse effects in rheumatology clinical trials II: the Rheumatology Common T Toxicity Criteria v.2.0. J Rheumatol 2007; 34(6):1401-1414) see the ISF for intensity/severity classification. Intensity options were: grade 1 (mild: asymptomatic or transient, short duration of <1 week); grade 2 (moderate: symptomatic, duration of 1-2 weeks); grade 3 (severe: prolonged symptoms, reversible, major functional impairment), and grade 4 (life-threatening: at risk of death, substantial disability, especially if permanent).

TABLE 6
Overview of Efficacy endpoints:

| | | | Time to 1st |
|---|---|---|---|
| Efficacy endpoints | Week 12 | By visit | achievement |
| Total AN count | | | |
| Percent change from baseline | Primary | Further | |
| Occurrence of at least 50, 75, 90, or | | | |
| 100 percentage change from | | | |
| baseline | | | |
| Draining fistula count | | | |
| Percent change from baseline¹ | Secondary | | |
| Occurrence of complete elimination¹ | Secondary | Further | |
| Total ANdT count | | | |
| Percent change from baseline | | Further | |
| HiSCR | Secondary | Further | Further |
| IHS4 | | | |
| IHS4 value² | Secondary | Further | |
| IHS4 category | | Further | |
| HASI | | | |
| HASI score² | Secondary | Further | |
| PGA | | | |
| PGA score 0 or 1 | Secondary | Further | Further |
| a ≥2-point reduction³ | | Further | |
| Rescue therapy | Further | Further: | Further: Time to 1st |
| | | | use |
| NRS30 | Secondary | Further | |
| Flare | Secondary | | |
| Patient-reported outcomes | | | |
| DLQI score² | Secondary | Further | |
| DLQI total score 0 or 1 | | Further | |
| DLQI improvement⁴ | | Further | |
| HiS-QoL Total Score² | Secondary | Further | |
| FACIT-Fatigue scale score² | | Further | |
| PGI-C score | | Further | |
| PGI-S score | | Further | |

¹Only for patients with baseline draining fistula count of ≥1
²Change from baseline
³Only for patients with baseline PGA score of ≥2
⁴DLQI improvement was defined as a ≥4-point reduction from baseline, only for patients with a DLQI ≥4 at baseline

### Adverse Events

An AE was defined as any untoward medical occurrence, including an exacerbation of a pre-existing condition, in a patient in a clinical investigation who received a pharmaceutical product. The event did not necessarily have to have a causal relationship with this treatment.

An AE could therefore be any unfavorable and unintended sign (including an abnormal laboratory finding), symptom, or disease temporally associated with the use of a medicinal product, whether or not considered related to the medicinal product.

The following should also have been recorded as an AE in the CRF and BI SAE form (if applicable): worsening of the underlying disease or of other pre-existing conditions; changes in vital signs, ECG, physical examination and laboratory test results, if they were judged clinically relevant by the investigator.

If such abnormalities already existed prior to trial inclusion, they were to be considered as baseline conditions and should have been collected in the electronic CRF (eCRF) only.

### Serious Adverse Events

A SAE was defined as any adverse event which resulted in death, was immediately life-threatening, resulted in persistent or significant disability/incapacity, required or prolonged patient hospitalization, was a congenital anomaly/birth defect, or was to be deemed serious for any other reason.

In accordance with EMA's initiative on Important Medical Events, BI had developed a list of further AEs, which by their nature, could always be considered “serious” even though they may not have met the criteria of an SAE as defined above. AEs considered “Always Serious” included cancers of new histology and exacerbations of existing cancer, which had to be classified as a serious event regardless of the duration between discontinuation of the drug and had to be reported.

### Causal Relationship of Adverse Event

Adverse events were judged in regard to their relationship to the investigational product. The medical judgement used to determine the relationship had to consider all relevant factors, including consistency with known pharmacology of the drug or with known AEs of the drug class, temporal relationship, de-challenge or re-challenge, confounding factors such as concomitant medication, concomitant diseases, and relevant history, lack of alternative explanation, indication of dose response. The investigator was asked to record a ‘yes’ if there was, in his/her judgement, a reasonable causal relationship between the investigational product administered and the adverse event or a ‘no’ if there was, in his/her judgement, no reasonable causal relationship between the investigational product administered and the adverse event.

### Adverse Events of Special Interest (AESI)

AESIs pre-specified in the CTP related to any specific AEs that had been identified at the project level as being of particular interest for prospective safety monitoring and safety assessment within this trial, e.g., the potential for AEs based on knowledge from other compounds in the same class. CTP-specified AESIs could be classified as serious or non-serious; all were to be reported on the SAE form in an expedited manner (within the same timeframe that applied to SAEs) to the sponsor's Pharmacovigilance Department.

The following AESIs were pre-specified:
Severe infections (according to RCTC grading): These included pneumocystis jirovecii, BK virus disease including PVAN, CMV, posttransplant lymphoproliferative disorder (EBV), progressive multifocal leucoencephalopathy, bartonellosis (disseminated only), blastomycosis, toxoplasmosis, coccidioidomycosis, histoplasmosis, aspergillosis (invasive only), candidiasis (invasive or pharyngeal), cryptococcosis, other invasive fungi (mucormycosis [zygomycosis, rhizopus, mucor, lichtheimia], scedosporium/pseudallescheria boydii, fusarium), legionellosis, Listeria monocytogenes (invasive only), tuberculosis, nocardiosis, non-tuberculous mycobacterium, salmonellosis (invasive only), HBV reactivation, herpes simplex (invasive only), herpes zoster, strongyloides (hyperinfection syndrome and disseminated forms only), paracoccidioides, Penicillium marneffei, sporothrix schenckii, cryptosporidium species (chronic only), microsporidiosis, leishmaniasis (visceral only), Trypanosoma cruzi infection (Chagas' disease)) (disseminated only), campylobacteriosis (invasive only), shigellosis (invasive only), vibriosis (invasive due to vibrio vulnificus), HCV progression (Winthrop K L, Novosad S A, Baddley J W, et al. Opportunistic infections and biologic therapies in immune-mediated inflammatory diseases: consensus recommendations for infection reporting during clinical trials and postmarketing surveillance. Ann Rheum Dis 2015; 74:2107-2116).
Hepatic injury: A hepatic injury was defined by the following alterations of hepatic laboratory parameters: • An elevation of Aspartate Aminotransferase (AST) and/or Alanine Aminotransferase (ALT)≥3 fold ULN, combined with an elevation of total bilirubin≥2 fold ULN measured in the same blood draw sample, or in samples drawn within 30 days of each other OR·ALT and/or AST elevations≥10 fold ULN. These lab findings constituted a hepatic injury alert, and any patients showing these lab abnormalities needed to be followed up according to the “Drug-Induced Liver Injury (DILI) checklist” provided in the ISF. In case of clinical symptoms of hepatic injury (icterus, unexplained encephalopathy, unexplained coagulopathy, right upper quadrant abdominal pain, etc.) without lab results (ALT, AST, total bilirubin) available, the investigator should have made sure that these parameters were analyzed, if necessary in an unscheduled blood test. User-defined AE concepts (UDAEC):

TABLE 7
Definition of UDAEC

| UDAEC | Category |
|---|---|
| Hypersensitivity | |
| Hypersensitivity ALL | All of the categories below |
| Anaphylactic reaction, narrow | Narrow SMQ “Anaphylactic reaction” |
| Angioedema, narrow | Narrow SMQ “Angioedema” |
| Hypersensitivity, narrow | Narrow SMQ “Hypersensitivity” |
| Infections (serious/severe, | |
| opportunistic) | |
| Infections ALL | All of the categories below |
| Opportunistic infections | Narrow SMQ “Opportunistic infections” |
| Tuberculosis infections | Sub-BleMQ “Tuberculosis related terms,” |
| | narrow BleMQ “Infections,” narrow sub-search |
| | 8.2 “Tuberculosis related terms” |
| Severe Infections | SOC “Infections and infestations” with |
| | AETOXGR ≥ 3 OR AESEV = “Severe.” |
| | Not Derived for studies where both severity |
| | variables are collected. |
| | SOC “Infections and infestations” - AEs |
| | of at least severe RCTC grade (≥3) or |
| | any severe events |
| Serious Infections | SOC “Infections and infestations” with |
| | [AECOND:AESER = 1] |
| Malignancies | |
| Malignant tumours | Narrow sub-SMQ “Haematological malignant |
| | Tumours,” Narrow sub-SMQ “Non- |
| | Haematological Malignant tumours” |
| Malignancies excluding | Sub-SMQ “Malignant tumours” excluding |
| non-melanoma skin | NMSC as defined above |
| Cancer (NMSC | |
| Torsades de Pointes | |
| Torsades de Pointes | Broad sub-SMQ “Torsade de pointes/QT |
| | prolongation |
| Suicidal ideation and | |
| behavior (SIB) | |
| Sub-SMQ “Suicide/self-injury” | Sub-SMQ “Suicide/self-injury” |
| DRESS | |
| Broad Drug reaction with | Broad SMQ “Drug reaction with |
| eosinophilia and | eosinophilia and |
| systemic symptoms syndrome | systemic symptoms syndrome” |
| (DRESS) | |
| Narrow DRESS | Narrow SMQ “Drug reaction with |
| | eosinophilia and |
| | systemic symptoms syndrome” |

### Treatments Administered:

TABLE 8
Test product spesolimab solution for infusion

| | |
|---|---|
| Substance: | Spesolimab |
| Pharmaceutical | Solution for infusion |
| formulation: | |
| Unit Strengths: | Spesolimab 450 mg/vial (concentration: |
| | 60 mg/mL, fill volume: 7.5 mL) |
| Route of Administration: | intravenous infusion |
| Posology: | Active arm: 1200 mg weekly at Weeks 0, 1, |
| | and 2 for a total loading dose of 3600 mg |

TABLE 9
Test product matching placebo solution for infusion

| | |
|---|---|
| Substance: | Placebo matching to spesolimab |
| Pharmaceutical formulation: | Solution for infusion |
| Unit Strengths: | Vials with fill volume: 7.5 mL |
| Route of Administration: | intravenous infusion |
| Posology: | Placebo arm: Placebo weekly at Weeks 0, |
| | 1, and 2 matching a total loading dose |
| | of 3600 mg spesolimab |

TABLE 10
Test product spesolimab solution for injection

| | |
|---|---|
| Substance: | Spesolimab |
| Pharmaceutical | Solution for injection |
| formulation: | |
| Unit Strengths: | Spesolimab 300 mg/pre-filled syringe |
| | (concentration: 150 mg/mL, fill volume: 2 mL) |
| Route of Administration: | Subcutaneous injection |
| Posology: | Active arm: 1200 mg every 2 weeks at |
| | Weeks 4, 6, 8, and 10 |

TABLE 11
Test product matching placebo solution for injection

| | |
|---|---|
| Substance: | Placebo matching to spesolimab |
| Pharmaceutical formulation: | Solution for injection |
| Unit Strengths: | Vials with a fill volume of 2 mL |
| Route of Administration: | Subcutaneous injection |
| Posology: | Placebo arm: Placebo every two weeks at |
| | Weeks 4, 6, 8, and 10 |

Individuals in the active arm receive a loading dose of 3600 mg spesolimab in 3 weekly intravenous administrations of 1200 mg spesolimab each (i.e., three infusions in total for the loading dose were to be administered at Weeks 0, 1, and 2). Individuals in the placebo arm receive matching placebo intravenous administrations at the same planned time-points. From Week 4 to Week 10 (both included) individuals receive s.c. injections of 1200 mg spesolimab or matching placebo every two weeks (i.e., four injections for a maximum of 4 visits in total [1 injection per visit] were to be administered).

This dose/regimen was selected to optimize the exposure of spesolimab to evaluate its efficacy in individuals with HS. The i.v. loading dose was selected to maximize the treatment response, the duration of response, and to reach a steady state sooner. A loading dose was able to also allow for an earlier onset of response prior to 12 weeks. The i.v. loading dose was followed by a s.c. maintenance dose given every 2 weeks. This dose was the maximum s.c. dose that could be conveniently given based on the injection volume and number of injections

TABLE 12
Doses and Dose Regimens

| | Frequency of | Frequency of | | Frequency of | Frequency of |
|---|---|---|---|---|---|
| Intravenous | intravenous | intravenous | Subcutaneous | subcutaneous | subcutaneous |
| dose (mg) | doses | administration | dose (mg) | doses | administration |
| 1200 | 3 | At 0, 1, 2 | 1200 | 4 | At 4, 6, 8, 10 |
| | | week intervals | | | or 12 weeks |
| | | | | | intervals - |

### Results:

### Primary Endpoint Analysis:

The key efficacy result was a marked positive signal for the reduction of abscess count and of draining fistula count at Week 12, in line with the hypothesized mechanism of action of spesolimab in HS. For the percent change from baseline in total abscess and inflammatory nodule (AN) count at Week 12, the difference in LS Mean (95% Cl) observed between spesolimab and placebo was −4.1 (−31.7, 23.4) based on the pre-specified MMRM analysis. Due to the small sample size, unbalanced baseline AN count between spesolimab and placebo, and extreme outliers in baseline AN count, an additional MMRM analysis with a categorical effect of baseline AN count was conducted after unblinding. The corresponding LS mean (95% CI) between spesolimab and placebo was −15.6 (−43.8, 12.6). In the percent change from baseline in draining fistula count at Week 12, the difference in LS Mean (95% CI) was −96.6 (−154.5, −38.8). See Table 13.

TABLE 13
MMRM estimates at Week 12 for percent change
from baseline (%) in total abscess and inflammatory
nodule (AN) count - SAF (EC-H MMRM)

| | % Change from Baseline | | | Comparison to Placebo | | |
|---|---|---|---|---|---|---|
| MMRM | N | LS Mean | SE | LS Mean | SE | 95% CI |
| Placebo | 14 | −34.7 | 11.1 | | | |
| Spesolimab | 30 | −38.8 | 7.5 | −4.1 | 13.7 | −31.7, 23.4 |

LS means, differences and confidence intervals were estimated by (REML) - based MMRM including the fixed, categorical effects of treatment at each visit, prior use of TNF inhibitor and the continuous effect of baseline at each visit as well as random effects of subject.
Primary estimand EC-H: data up to use of rescue therapy are included for analysis, and data post the use are censored. Unstructured covariance matrix was used.

Because of the small sample size, imbalanced baseline AN count between spesolimab and placebo, and extreme outliers in baseline AN count, an additional MMRM analysis with a categorical effect of baseline AN count was conducted after unblinding. The corresponding LS mean (95% CI) between spesolimab and placebo was-15.6 (−43.8, 12.6)

### Secondary Efficacy Endpoints:

The continuous secondary endpoints at Week 12 were analyzed using a MMRM approach based upon the estimand concept and model specification as described for the primary endpoint. The binary secondary endpoints were evaluated descriptively by treatment arm via frequency tables (with 95% confidence interval) based on the safety analysis set (SAF). If needed, a logistic regression model on binary endpoint at Week 12 was to be applied as well. The model was to include treatment and stratification factor (TNFi-naive population versus TNFi-failure population) as two categorical variables. As in the case of the primary estimand, any binary data collected after use of any rescue therapy were to be censored and then imputed using the non-response imputation (NRI) method.

The continuous further endpoints were evaluated in the same manner as described for primary endpoint. The binary further endpoints were evaluated in the same manner as described for the secondary binary endpoints. For time to event endpoints, such as for time to first achieving HiSCR, Kaplan Meier-estimates of the survival/failure probabilities, as well as the median time-to-event, were provided. 95% confidence intervals were provided. Questionnaires were descriptively summarized by visit.

### Secondary Endpoint: An Achievement of HiSCR at Week 12

Hidradenitis Suppurativa Clinical Response (HiSCR) is defined as at least a 50% reduction in the total AN count with no increase in abscess count and no increase in dT count relative to baseline.

At Week 12, a numerically higher proportion of individuals achieved HiSCR in the spesolimab group compared to those in the placebo group; the risk difference (95% CI) was 13.8 (−12.9, 33.9); see Table 14 below.

TABLE 14
Proportion of patients with achievement
of HiSCR at Week 12 - SAF (EC-H-NRI)

| | | | | Difference | |
|---|---|---|---|---|---|
| NRI | N | n | % | vs. Placebo | 95% CI |
| Placebo | 17 | 3 | 17.6 | | −12.9, 33.9 |
| Spesolimab | 35 | 11 | 31.4 | 13.8 | |

Note:
The denominator for percentages and proportions is the number of patients in the analysis set.
95% CI for each treatment is calculated by Wilson method and 95% CI for treatment difference is calculated by Chan and Zhang method.
Primary estimand EC-H: Data up to use of rescue therapy are included for analysis, and data post the use are censored

The consistency of the treatment effect was investigated in the same predefined subgroups as for the primary endpoint. In general, the results for the secondary endpoint proportion of patients with an achievement of HISCR at Week 12 were comparable across the subgroups. The patients presenting dT at baseline showed better response in HiSCR in the spesolimab group than in the placebo group. However, the treatment effects estimates were generally comparable to the estimates of the primary analysis. Overall, taking the small size of many of the subgroups into account, no evidence was found for a different effect of spesolimab compared to placebo on the treatment of HS across all subgroups.

Secondary Endpoints: Other than HISCR

The primary analysis method for the secondary endpoints was the estimand EC-H. With the EC estimand any values observed after use of rescue medication for the purpose of disease worsening were to be censored, however, no patients used rescue medication.

The continuous further endpoints were evaluated in the same manner as described for primary endpoint. The binary further endpoints were evaluated in the same manner as described for the secondary binary endpoints. For time to event endpoints, such as for time to first achieving HiSCR, Kaplan Meier estimates of the survival/failure probabilities, as well as the median time-to-event, were provided. 95% confidence intervals were provided. Questionnaires were descriptively summarized by visit.

The results of additional secondary endpoints other than HISCR are presented in Table 15.

A Percent Change from Baseline in dT Count at Week 12.

At Week 12, the spesolimab group showed better clinical improvement than the placebo group; comparing to the placebo group, the difference in LS Mean (95% CI) was-96.6 (−154.5, −38.8); see Table 15, below. The reduction in dT count in the spesolimab group seemed to reach its near maximum as early as Week 2 to Week 4 and seemed sustained until Week 12.

An Absolute Change from Baseline in IHS4 Value at Week 12:

At Week 12, the spesolimab group showed a better clinical improvement than the placebo group; comparing to the placebo group, the difference in LS Mean (95% CI) was −13.9 (−25.6, −2.3); see Table 15, below. Of note, the mean (SD) of IHS4 value at baseline was 40.0 (34.2) in the placebo group and 28.2 (22.3) in the spesolimab group.

An Absolute Change from Baseline in HASI Score at Week 12:

At Week 12, the spesolimab group showed better clinical improvement than the placebo group; comparing to the placebo group, the difference in LS Mean (95% CI) was −19.8 (−36.9, −2.7); see Table 15, below. Of note, the mean (SD) of HASI score at baseline was 82.1 (79.1) in the placebo group and 59.8 (49.5) in the spesolimab group.

### An Achievement of PGA Score of 0 or 1 at Week 12:

At Week 12, the achievement of PGA score of 0 or 1 was observed in the spesolimab group but not in the placebo group; comparing to the placebo group, the risk difference (95% CI) was 5.7 (−13.2, 18.6); see Table 15, below.

### An Achievement of NRS30 in Patient's Global Assessment of HS Pain at Week 12:

At Week 12, in a higher proportion of individuals, the achievement of NRS30 in Patient's Global Assessment of HS Pain was observed in the spesolimab group than in the placebo group; comparing to the placebo group, the risk difference (95% CI) was 17.0 (−6.7, 33.8); see Table 15, below.

### An Occurrence of Complete Elimination of dT at Week 12:

At Week 12, in a higher proportion of individuals, the occurrence of complete elimination of dT was observed in the spesolimab group than in the placebo group; comparing to the placebo group, the risk difference (95% CI) was 18.3 (−7.9, 37.5); see Table 15, below.

An Occurrence of at Least One Flare (Defined as at Least 25% Increase in AN Count with a Minimum Increase of 2 Relative to Baseline) Up to Week 12:

At Week 12, for the occurrence of at least one flare, comparing the spesolimab group to the placebo group, the risk difference (95% CI) was −9.1 (−33.1, 8.9); see Table 15, below.

An Absolute Change from Baseline in DLQI Score at Week 12:

At Week 12, the clinical improvement in both treatment groups was comparable; comparing to the placebo group, the difference in LS Mean (95% Cl) was −0.1 (−4.4, 4.3); see Table 15, below.

An Absolute Change from Baseline in HIS-QoL Total Score at Week 12:

At Week 12, the clinical improvement in both treatment groups was comparable; comparing to the placebo group, the difference in LS Mean (95% CI) was 1.5 (−6.9, 9.8); see Table 15, below.

TABLE 15
Additional secondary endpoints other than HiSCR
MMRM estimates at Week 12 for percent change from baseline (%) in dT count - SAF (EC-H-MMRM)

| | % Change from Baseline | | | Comparison to Placebo | | |
|---|---|---|---|---|---|---|
| MMRM | N | LS Mean | SE | LS Mean | SE | 95% CI |
| Placebo | 13 | −56.6 | 23.0 | | | |
| Spesolimab | 24 | −40.1 | 16.8 | −96.6 | 28.6 | −154.5, −38.8 |

MMRM estimates at Week 12 for absolute change from baseline in IHS4 value - SAF (EC-H-MMRM)

| | Absolute Change from Baseline | | | Difference v Placebo | | |
|---|---|---|---|---|---|---|
| MMRM | N | LS Mean | SE | LS Mean | SE | 95% CI |
| Placebo | 14 | 4.9 | 4.7 | | | |
| Spesolimab | 30 | −9.0 | 3.2 | −13.9 | 5.8 | −25.6, −2.3 |

MMRM estimates at Week 12 for absolute change from baseline in HASI score - SAF (EC-H-MMRM)

| | N | LS Mean | SE | LS Mean | SE | 95% CI |
|---|---|---|---|---|---|---|
| Placebo | 14 | −3.8 | 6.9 | | | |
| Spesolimab | 30 | −23.6 | 4.7 | −19.8 | 8.5 | −36.9, −2.7 |

Proportion of patients with occurrence of complete elimination of dT at Week 12 - SAF (EC-H-NRI)

| | N | n | % | Difference vs Placebo | 95% CI |
|---|---|---|---|---|---|
| Placebo | 15 | 1 | 6.7 | | |
| Spesolimab | 28 | 7 | 25.0 | 18.3 | −7.9, 37.5 |

Proportion of patients with occurrence of at least one flare at Week 12 - SAF (EC-H-NRI)

| | N | n | % | Difference vs Placebo | 95% CI |
|---|---|---|---|---|---|
| Placebo | 17 | 3 | 17.6 | | |
| Spesolimab | 35 | 3 | 8.6 | −9.1 | −33.1, 8.9 |

Proportion of patients with achievement of at least 30% reduction from baseline in NRS at Week 12 - SAF (EC-H-NRI)

| | N | n | % | Difference vs Placebo | 95% CI |
|---|---|---|---|---|---|
| Placebo | 17 | 1 | 5.9 | | |
| Spesolimab | 35 | 8 | 22.9 | 17.0 | −6.7, 33.8 |

Proportion of patients with achievement of PGA score of 0 or 1 at Week 12 - SAF (EC-H-NRI)

| | N | n | % | Difference vs Placebo | 95% CI |
|---|---|---|---|---|---|
| Placebo | 17 | 0 | 0 | | |
| Spesolimab | 35 | 2 | 5.7 | 5.7 | −13.2, 18.6 |

MMRM estimates at Week 12 for absolute change from baseline in DLQI Score - SAF (EC-H-MMRM)

| | Absolute Change from Baseline | | | Difference v Placebo | | |
|---|---|---|---|---|---|---|
| MMRM | N | LS Mean | SE | LS Mean | SE | 95% CI |
| Placebo | 14 | −2.8 | 1.8 | | | |
| Spesolimab | 30 | −2.8 | 1.2 | −0.1 | 2.2 | −4.4, 4.3 |

MMRM estimates at Week 12 for absolute change from baseline in HiS-QoL Total Score - SAF (EC-H-MMRM)

| | Absolute Change from Baseline | | | Difference v Placebo | | |
|---|---|---|---|---|---|---|
| MMRM | N | LS Mean | SE | LS Mean | SE | 95% CI |
| Placebo | 14 | 4.4 | 3.3 | | | |
| Spesolimab | 25 | 5.8 | 2.4 | 1.5 | 4.1 | −6.9, 9.8 |

Note:
LS means, differences and confidence intervals were estimated by (REML)-based MMRM including the fixed, categorical effects of treatment at each visit, prior use of TNF inhibitor and the continuous effect of baseline at each visit as well as random effects of subject.
Primary estimand EC-H: Data up to use of rescue therapy are included for analysis, and data post the use are censored.
Only patients with baseline draining fistulas >=1 are included.
Unstructured covariance matrix was used.

### Further Endpoints Related to Lesion Counts Including Abscess, Inflammatory Nodule, Non-Inflammatory Nodule, and Draining Fistula Count

After Week 2, a larger proportion of individuals in the spesolimab group than in the placebo group had at least 50 percentage change from baseline in AN count. The values for both treatment groups diverged from Week 4 onwards. In the spesolimab group, the response rates increased afterwards to above 50%, with the largest response of 57.1% (20 patients) seen at Week 6. This response level was generally sustained up to Week 12. At Week 12, 48.6% of patients (17 patients) in the spesolimab group and 35.3% of patients (6 patients) in the placebo group achieved at least 50 percentage change from baseline in AN count.

The proportion of individuals with at least 75 percentage change from baseline in AN count over time, based on the EN-H-NRI approach (data not shown). The curves for the 2 treatment groups separated from Week 2 onwards. In the spesolimab group, the response rates increased afterwards to 40%, with the largest response of 40.0% (14 patients) seen at Week 8, with a constantly higher proportion of responders in the spesolimab group than in the placebo group up to Week 10. Afterwards, the response rates in the spesolimab group decreased, with the similar response seen in both treatment groups at Week 12. At Week 12, 25.7% of patients (9 patients) in the spesolimab group and 29.4% of patients (5 patients) in the placebo group had achieved at least 75 percentage change from baseline in AN count.

The proportion of individuals with at least 90 percentage change from baseline in AN count over time, based on the EN-H-NRI approach was measured. Generally, compared with the placebo group, a slightly higher proportion of patients in the spesolimab group had at least 90 percentage change from baseline in AN count over time.

The proportion of patients with at least 100 percentage change from baseline in AN count over time, based on the EN-H-NRI approach was measured. Generally, compared with the placebo group, a slightly higher proportion of individuals in the spesolimab group had at least 100 percentage change from baseline in AN count over time. The response in both treatment groups was similar to the occurrence of at least 90 percentage change from baseline in AN count over time.

The proportion of individuals with occurrence of complete elimination of dTs up to Week 12 was measured. After Week 2, a larger proportion of individuals in the spesolimab group than in the placebo group had complete elimination in dTs. The values for both treatment groups diverged from Week 2 onwards. In the spesolimab group, the response rates increased up to 32.1% (9 patients) seen at Weeks 4 and 6. This response level was slightly decreased to 25.0% (7 patients) at Week 8 and afterwards sustained up to Week 12.

The percent change from baseline in ANdT count up to Week 12 was measured (data not shown). The curves for the 2 treatment groups separated after Week 1. In the spesolimab group, the LS Mean (95% CI) for percent change from baseline in total ANdT count reached up to −44.0 (−59.5, −28.5) at Week 6, with a larger response in the spesolimab group than in the placebo group after Week 1 up to Week 8. Afterwards, the response in the spesolimab group decreased, with the similar response seen in both treatment groups at Week 10. At Week 12, comparing the spesolimab group to the placebo group, the difference in LS Mean (95% CI) for the percent change from baseline in ANdT count was −8.8 (−32.3, 14.7).

The estimated probability of achieving HiSCR was larger in the spesolimab group than in the placebo group based on observed cases was measured (data not shown). The separation of the estimated probability between spesolimab and placebo started 3 weeks after randomization and was maintained throughout the trial. Individuals in the spesolimab group achieved the response, starting at Week 1, gradually increasing the probability up to 71.4% by Week 11. In the placebo group, at Week 1 the probability to achieve a response was 5.9% and at Week 12 the probability was 47.1%.

The absolute change from baseline in IHS4 value up to Week 12 was measured. In the spesolimab group, the onset of improving in IHS4 value was observed from Week 1. In the spesolimab group, the LS Mean (95% CI) for absolute change from baseline in IHS4 value reached afterwards to −11.1 (−17.7, −4.5) seen at Week 6, and the benefits of IHS4 value was sustained until Week 12.

The proportion of patients with a IHS4 category over time was measured. Under the OC estimand, in the spesolimab group, the largest proportion of patients was 77.1% (27/35) of patients with severe at baseline. At Week 12, the largest proportion of patients was 50.0% (15/30) with severe still while 40% (12/30) with moderate, with the distribution of patients slightly shifting over time to lower scores. In the placebo group, the largest proportion of patients was 88.2% (15/17) of patients with severe at baseline and 78.6% (11/14) of patients with severe at Week 12, without the distribution of patients shifting during the course of the trial. The IHS4 category in the spesolimab group showed tendency of improvement.

The absolute change from baseline in HASI score up to Week 12 was measured. Compared to the placebo group, the HASI score in the spesolimab group started to improve from Week 2, with a near maximum effect reached by Week 6. The benefit in HASI score for the spesolimab group was sustained until Week 12.

The estimated probability of achieving PGA score of 0 or 1 was low and comparable in both treatment groups based on observed cases. The separation of the estimated probability between spesolimab and placebo started from 3 weeks after randomization, however, the difference was not increased afterwards. Individuals in the spesolimab group achieved the response starting at Week 3 with a probability of 5.7% (2 patients) by Week 8 and at Week 12 the probability was 8.6% (3 patients). In the placebo group, from 9 weeks after randomization, the probability to achieve a response was 5.9% (1 patient) by Week 12.

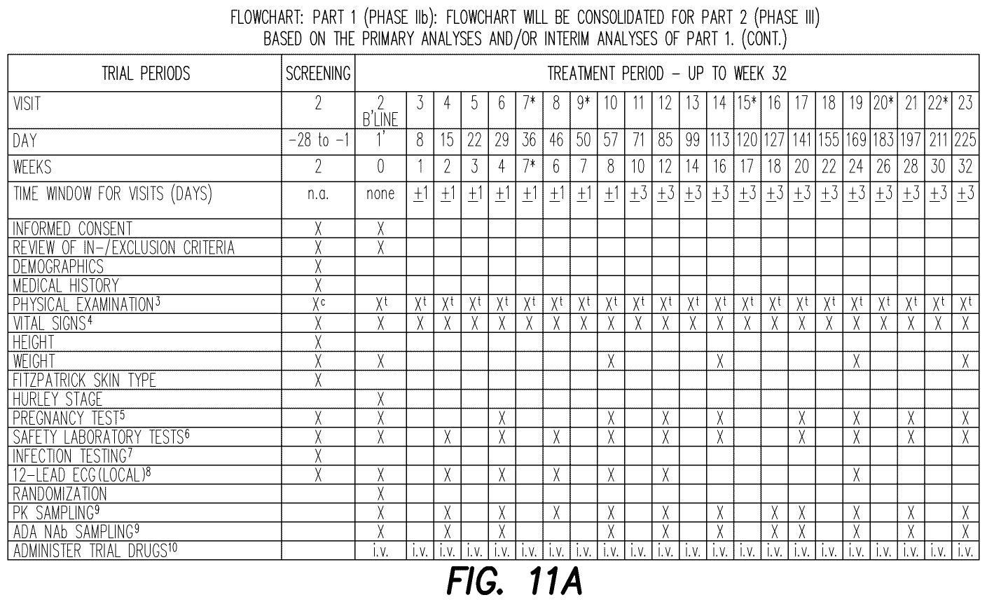

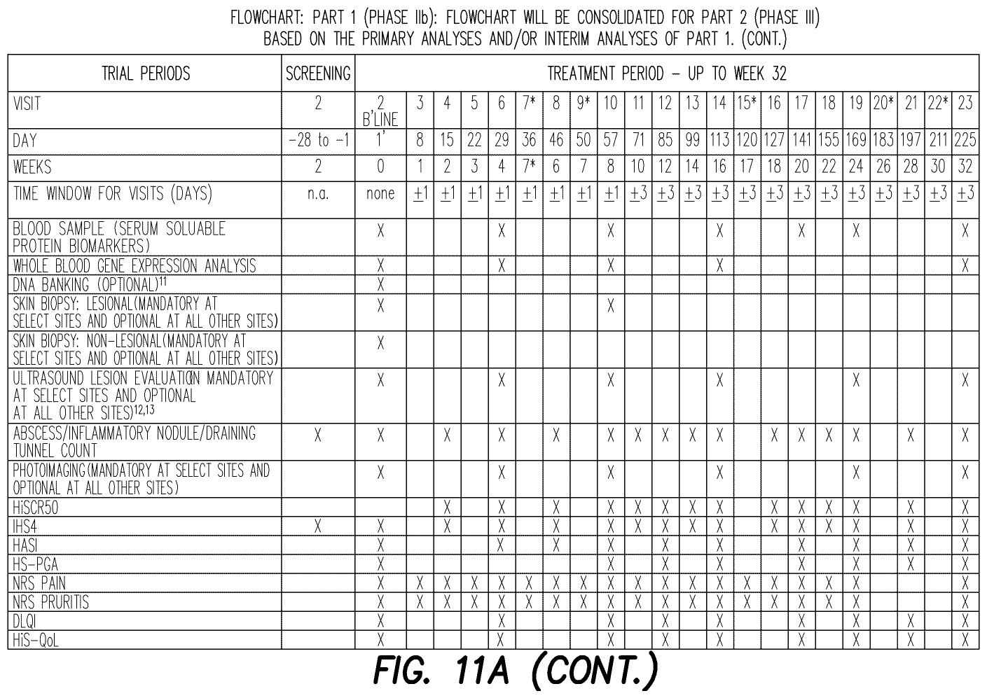

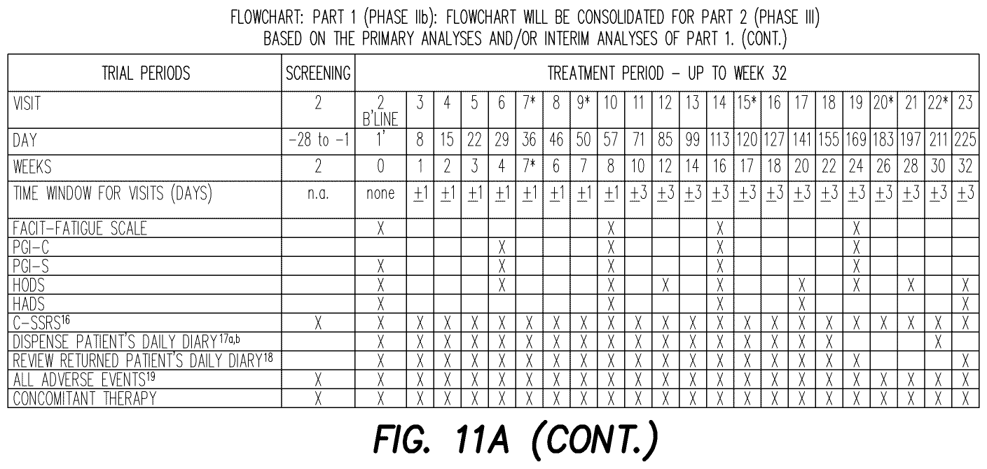

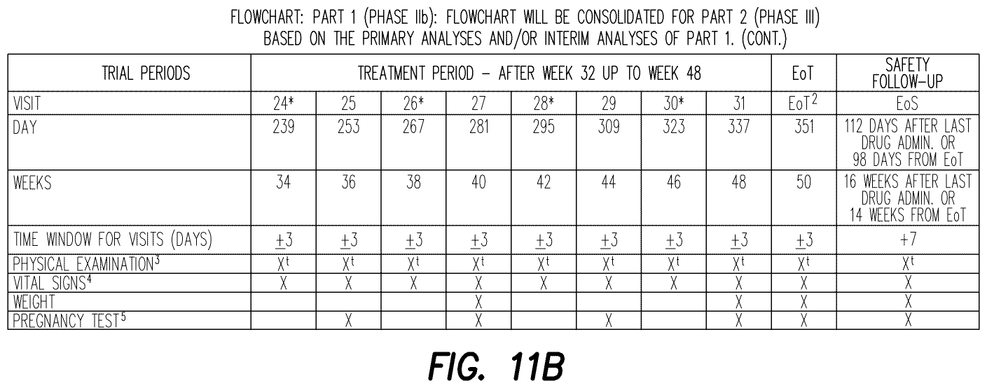

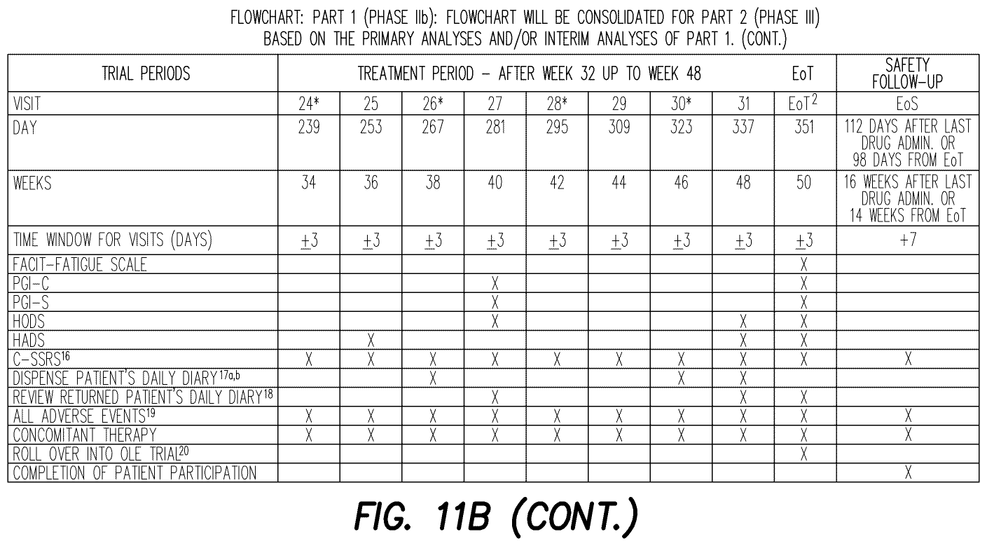

In both treatment groups, the proportion of patients with PGA score of 0 or 1 over time was low. The proportion of patients with achievement of PGA score of 0 or 1 over time is presented in FIG. 11.1.3.3: 2. Under the EC-H-NRI estimand, the curves for spesolimab and placebo did not separate.

A slightly higher proportion of patients showed achieving of a≥2-point reduction in PGA score at Week 12 in the spesolimab group compared with the placebo group. The proportion of patients with achievement of a≥2-point reduction in PGA score over time (only for patients with baseline PGA score of ≥2) is presented in FIG. 11.1.3.3: 3. Under the EC-H-NRI estimand, the curves for spesolimab and placebo showed the largest separation at Week 6 in favor of spedolimab, however, the difference was decreased afterwards.

No patients used rescue therapy in both treatment groups.

The proportion of patients with achievement of NRS 30 over time was measured (data not shown). The separation of the curve between spesolimab and placebo started from Week 1 in favor of spesolimab and sustained until Week 12 although the difference was temporally decreased from Week 2 to 4. The risk difference between spesolimab and placebo of 22.9% was the largest at Week 1.

The absolute change from baseline in DLQI Score up to Week 12 was measured. The mean and median DLQI scores at baseline were comparable between the treatment groups. In both treatment groups, the mean absolute change from baseline in DLQI score decreased from Week 1 and reached a maximum decrease of −2.8 at Week 12.

Overall, the proportion of patients with an achievement of DLQI total score 0 or 1 was low and comparable between the treatment groups.

The proportion of patients with achievement of DLQI improvement up to Week 12 was measured (data not shown). Overall, the proportion of patients with an achievement of DLQI improvement was comparable through the trial. In both treatment groups, the proportion of patients with achievement of DLQI improvement increased from Week 1. In the spesolimab group, the proportion of patients with achievement of DLQI improvement reached a maximum of 44.1% (15 patients) at Week 8 and sustained until Week 12. In the placebo group, the proportion of patients with achievement of DLQI improvement gradually increased further after Week 1 until Week 12 and reached a maximum of 46.7% (7 patients) at Week 12.

The absolute change from baseline in HIS-QOL Total score up to Week 12 is comparable in both treatment groups. In both treatment groups, an absolute change from baseline in HIS-QOL Total Score increased from Week 1 and reached a maximum (spesolimab 6.8, placebo 6.1) at Week 8.

The FACIT-Fatigue score improved at Week 12 by 2.7 points in the spesolimab group and by 3.7 points in the placebo group. In the spesolimab group, the improvement in the FACIT-Fatigue score seemed to reach its near maximum as early as Week 1 and seemed sustained until Week 12. In the placebo group, the FACIT-Fatigue score worsened by 1.9 point at Week 1 and afterwards improved until Week 12, where an improvement of 3.7 points was seen. Details for the adjusted mean absolute change from baseline in the FACIT-Fatigue score over time, based on the EC-H-MMRM approach was used for the analysis (data not shown).

The proportion of patients with a PGI-C score over time was measured. Under the OC estimand, in the spesolimab group, the largest proportion of patients was 68.8% (22/32) of patients with PGI-C score 3 (No change) at Week 1 and 50.0% (15/30) with PGI-C score 2 (A little better) at Week 12, with the distribution of patients shifting over time to lower scores. In the placebo group, the largest proportion of patients was 70.6% (12/17) of patients with PGI-C score 3 (No change) at Week 1 and 50.0% (7/14) of patients with PGI-C score 3 (No change) at Week 12, without the distribution of patients shifting during the course of the trial. The PGI-C score in the spesolimab group showed tendency of improvement.

At baseline, in the spesolimab group, the largest proportion of patients was 48.6% (17/35) of patients with PGI-S score 3 (Severe), while in the placebo group, the largest proportion of patients was 47.1% (8/17) of patients with PGI-S score 2 (Moderate). Under the OC estimand, in the spesolimab group, the largest proportion of patients was 46.9% (15/32) of patients with PGI-S score 3 (Severe) at Week 1 and 37.9% (11/29) with PGI-S score 1 (Mild) at Week 12, with the distribution of patients shifting over time to lower scores. In the placebo group, the largest proportion of patients was 56.3% (9/16) of patients with PGI-S score 2 (Moderate) at Week 1 and 50.0% (7/14) of patients with PGI-S score 2 (Moderate) at Week 12, without the distribution of patients shifting during the course of the trial. The PGI-S score in the spesolimab group showed tendency of improvement.

### Safety

The proportions and incidence rates of patients with any AE were comparable between the placebo and the spesolimab treatment group. Around four-fifths of patients reported at least one AE (placebo: 14 patients, 87.5%; spesolimab: 28 patients, 77.8%). The incidence rate of drug-related AEs was higher in the spesolimab treatment group (15 patients, 41.7%) than in the placebo treatment group (3 patients, 18.8%), largely due to injection site reactions. There was no incidence of AEs leading to treatment discontinuation, other significant AEs, and SAEs in the spesolimab group. In the placebo group, 1 patient (6.3%) was reported with AEs leading to treatment discontinuation, other significant AEs, and SAEs, for each. No AEs leading to death, no severe AEs, and no protocol-defined AEs of special interest (systemic hypersensitivity including infusion reaction and anaphylactic reaction, severe infections [according to RCTC grading], opportunistic and Mycobacterium tuberculosis infections, or hepatic injury) were reported (Table 16). The proportions and incidence rates of patients with any AE was 65.4% in loading stage and 80.8% in overall period.

TABLE 16
Overall summary of AEs - safety analysis set

| | Placebo | | Spesolimab | | Overall Total | |
|---|---|---|---|---|---|---|
| Category of AE | N (%) | | N (%) | | N (%) | |
| Number of patients | 16¹ | (100.0) | 36¹ | (100.0) | 52 | (100.0) |

| Total overall time at risk | 4.1 | | 9.8 | | 14.0 | |
|---|---|---|---|---|---|---|
| [patient-years] | | | | | | |
| Patients with any AE | 14 | (87.5) | 28 | (77.8) | 42 | (80.8) |

| Patients with severe AEs | 0 | | 0 | | 0 | |
|---|---|---|---|---|---|---|
| (RCTC grade 3 or 4) | | | | | | |
| Patients with investigator | 3 | (18.8) | 15 | (41.7) | 18 | (34.6) |
| defined drug-related AEs | | | | | | |

| Patients with AEs leading to | 1 | (6.3) | 0 | 1 | (1.9) |
|---|---|---|---|---|---|
| discontinuation of trial drug | | | | | |
| Patients with investigator | 0 | | 0 | 0 | |
| defined AEs of special interest² | | | | | |
| Patients with other significant | 1 | (6.3) | 0 | 1 | (1.9) |
| AEs (according to project def.) | | | | | |
| Patients with SAEs | 1 | (6.3) | 0 | 1 | (1.9) |

¹For safety reporting, patient randomized to receive placebo took spesolimab during the study is considered in spesolimab arm instead of assigned group. Patient randomized to receive spesolimab took placebo during the study is considered still in spesolimab arm.
²Systemic hypersensitivity including infusion reaction and anaphylactic reaction, severe infections [according to RCTC grading], opportunistic and mycobacterium tuberculosis infections, or hepatic injury

In the overall period, the most frequently reported events on the system organ class (SOC) level was infections and infestations, followed by general disorders and administration site conditions, skin and subcutaneous tissue disorders, and nervous system disorders. The proportions of patients and incidence rates were generally balanced between the 2 treatment groups for most SOCs except for general disorders and administration site conditions and gastrointestinal disorders. The frequencies and incidence rates of general disorders and administration site conditions were lower in the placebo group (2 patients [12.5%]) than in the spesolimab group (12 patients [33.3%]) and also in gastrointestinal disorders; in the placebo group (1 patient [6.3%]) than in the spesolimab group (6 patients [16.7%]). All cases were categorized as mild intensity. On the preferred term (PT) level, the most frequently reported AEs overall total in the overall period was headache (overall: 7 patients [13.5%]; spesolimab: 4 patients [11.1%], placebo: 3 patients [18.8%]), followed by nasopharyngitis (overall: 6 patients [11.5%]; spesolimab: 3 patients [8.3%], placebo: 3 patients [18.8%]). All other PTs were reported for at most 4 patients overall. On the PT level, investigator-defined drug-related AEs which were reported more frequently in the spesolimab group as compared to the placebo group were nausea (spesolimab: 2 patients [5.6%], placebo: 0 patient [0%]), acne (spesolimab: 2 patients [5.6%], placebo: 0 patient [0%], injection site pain (spesolimab: 3 patients [8.3%], placebo: 1 patient [6.3%]), injection site erythema (spesolimab: 3 patients [8.3%], placebo: 0 patient [0%]), and fatigue (spesolimab: 2 patients [5.6%], placebo: 0 patient [0%]). The incidence of other individual PTs was generally comparable between the treatment groups. Trial statistical analysis plan-defined AE groupings by medical concepts, defined as user-defined AE concepts, were based on standardized MedDRA queries (SMQs) or BI-customized systematic MedDRA queries (BIcMQs) if no SMQs were available. The AEs categorized in the SMQs angioedema, depression (excl suicide and self-injury), and suicide/self-injury were reported for 1 patient each, while hypersensitivity was reported for 4 patients. No meaningful numerical differences were identified between treatment groups considering the small size of the treatment groups and the 2:1 randomization. No AEs categorized in the SMQs anaphylactic reactions, opportunistic infections, severe infections, serious infections, malignant tumours, NMSC, and DRESS, or AEs categorized in the BIcMQs tuberculosis infections were reported.

### Anti-Drug Antibodies and Neutralizing Antibodies

Anti-drug antibody (ADA) response was determined for every patient.

Immunogenicity of spesolimab was assessed using a multi-tiered approach. Briefly, all samples were first analyzed in the ADA screening assay, and only those found to be putative positive in the ADA screening assay were assessed in the ADA confirmatory assay. Only samples that confirmed positive were then titrated (to obtain a titer value). Neutralizing antibody (NAb) response was also determined for every confirmed ADA positive patient.

In total, 52 patients were randomized to receive spesolimab or placebo (2:1 ratio) in this trial. Thirty-five patients received spesolimab at Week 0, and 17 patients received placebo at Week 0. Three patients in the spesolimab treatment group and 1 patient in the placebo treatment group prematurely discontinued trial medication.

For the ADA assessment, baseline is defined as the trial visit day when the ADA sample was collected prior to the initial spesolimab treatment. Overall, out of 34 spesolimab-treated ADA evaluable patients, 6 patients (17.6%) were ADA positive after treatment, and 27 patients (79.4%) were ADA negative through the trial duration. The 6 ADA-positive patients after treatment were also ADA positive at the last sample collected.

The majority (66.7%) of the ADA-positive patients were also NAb positive. In the ADA positive HS patients, ADA was detected at a median onset time of 6.00 weeks and reached maximum titer at a median time of 12.0 weeks. Maximum titer occurred at the last sample collected. In NAb-positive patients, NAb was detected at a median onset time of 7.90 weeks. Given the low number of NAb-positive patients in this trial, definitive conclusions on immunogenicity and its impact on PK was not explored. Due to the short duration of this trial, the ADA/NAb incidence may be underestimated.

In treatment-induced ADA-positive HS patients, ADA was detected at a median onset time of 6.00 weeks and reached maximum titer at a median time of 12.0 weeks. Maximum titer occurred at the last sample collected. In NAb-positive patients, NAb was detected at a median onset time of 7.90 weeks.

### Discussion

The prespecified primary analysis of this small exploratory study resulted in little difference between treatment groups for the percent change from baseline at Week 12 in total count of abscesses and inflammatory nodules. However, a positive signal on secondary outcomes emerging as clinically important in HS was observed at Week 12: in percent change from baseline in dT count, in absolute change from baseline in IHS4 (dT, abscess, and inflammatory weighed counts), and in absolute change from baseline in HASI (severity and extent of HS). A positive trend was also observed in patients achieving HiSCR and in patients achieving at least a 30% reduction from baseline in NRS Patient's Global Assessment of HS pain.

In the percent change from baseline in dT count at Week 12, the difference in LS Mean (95% CI) was −96.6 (−154.5, −38.8). In the absolute change from baseline in IHS4 value at Week 12, the difference in LS Mean (95% CI) was −13.9 (−25.6, −2.3). In the absolute change from baseline in HASI score, the difference in LS Mean (95% CI) was −19.8 (−36.9, −2.7). Although the endpoints containing inflammatory nodules showed less efficacy, the HS-related outcomes including dT count, IHS4 value, and HASI score indicated an improvement in favor of spesolimab.

The achievement of NRS30 in Patient's Global Assessment of HS Pain was observed in a higher proportion of patients in the spesolimab group than in the placebo group; the risk difference (95% CI) was 17.0 (−6.7, 33.8). The achievement of PGA score of 0 or 1 at Week 12 was observed in the spesolimab group but not in the placebo group. For the absolute change from baseline in DLQI Score at Week 12 and the absolute change from baseline in HIS-QOL Total Score at Week 12, the numerical change at Week 12 in both treatment groups was comparable.

In the percentage change from baseline in both AN count and ANdT count over time, treatment with spesolimab showed the tendency of improvement up to Week 6, however the treatment effect was not maintained up to Week 12. The outcomes of HS-related endpoints including dT count, HiSCR, IHS4, and HASI indicated an improvement in favor of spesolimab, while the PGA-related endpoints did not show efficacy of spesolimab during the course of trial. The results of subjective assessments including DLQI, HIS-QOL, and FACIT Fatigue did not show efficacy of spesolimab, while the outcomes related to NRS30, PGI-C, and PGI-S showed tendency of improvement in the spesolimab group compared with the placebo group

Overall, in trial 1368-0052, spesolimab was well tolerated, generally in line with what seen in previous spesolimab-trials in other indications. The results obtained in this proof of clinical concept study, in particular on a high unmet medical need (i.e., dT count reduction) in line with the expected mechanism of action of spesolimab in HS, support further development of spesolimab in HS.

Analysis of an Open-Label Study of Spesolimab Following a Randomized, Placebo-Controlled, Phase IIa Trial in Patients with Moderate-to-Severe HS Through Week 50:

OBJECTIVE: To describe 1-year interim safety and efficacy results with open-label spesolimab treatment.

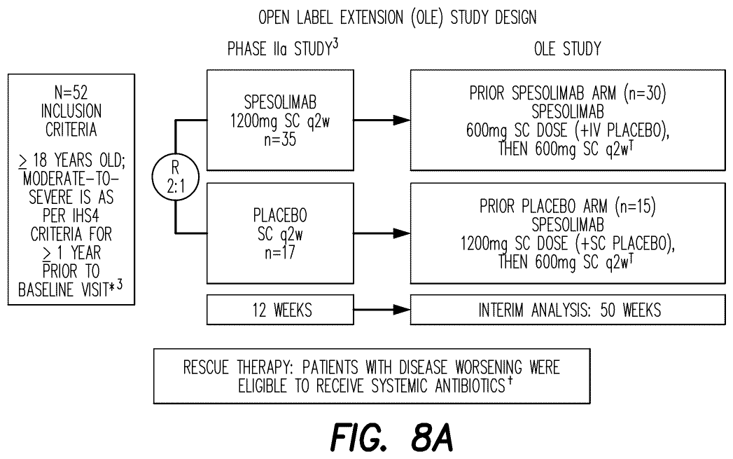

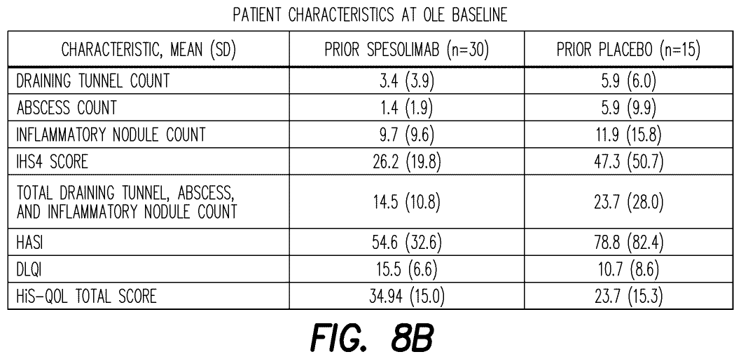

DESIGN: Phase IIa and OLE Study Design: Week 24 (Week 12 of the OLE), patients could have their dose of spesolimab increased to 1200 mg SC q2w if required (based on responder status at start of OLE and HS-PGA severity change from start of OLE). HS disease worsening was defined as a 150% increase in abscess and inflammatory nodule count from baseline. Baseline refers to last measurement before start of spesolimab, i.e., baseline of Phase IIa trial in prior spesolimab arm or baseline of OLE study in prior placebo arm (FIG. 8A). Patients eligible for inclusion also had HS lesions in ≥2 distinct body areas; a total abscess and inflammatory nodule (AN) count≥5; a total draining tunnel count≤20; were biologic-naïve or had failed on previous TNF-α inhibitor treatment for HS and had an inadequate response to oral antibiotics for HS in the past year (FIG. 8B).

RESULTS: Spesolimab was well-tolerated through 50 weeks with a safety profile in line with the Phase IIa study and other spesolimab trials. Safety analyses included patients with 21 year of spesolimab treatment (n=20) and those who prematurely discontinued treatment (n=25). Mean (SD) duration of exposure: 39.1 (24.1) weeks in prior spesolimab arm, 42.9 (28.4) weeks in prior placebo arm.

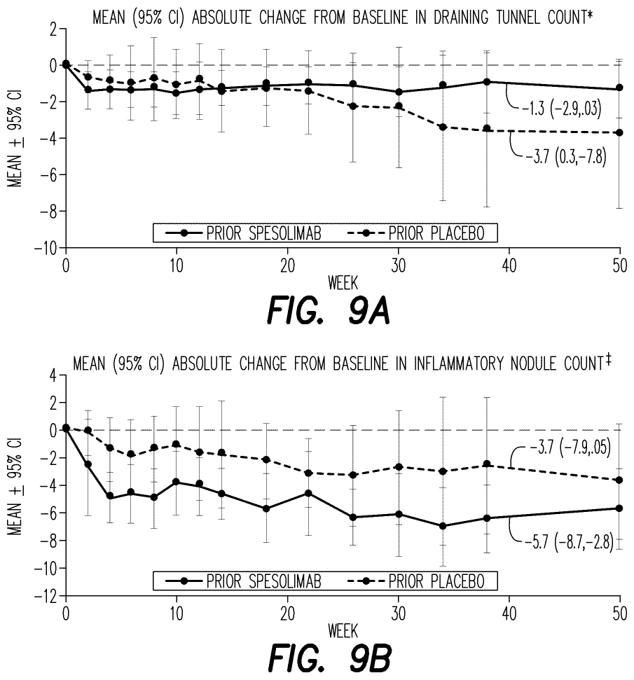

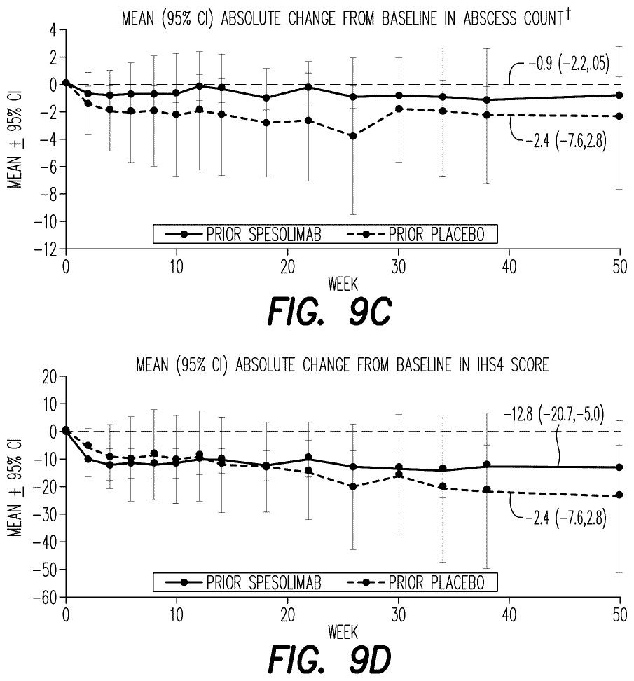

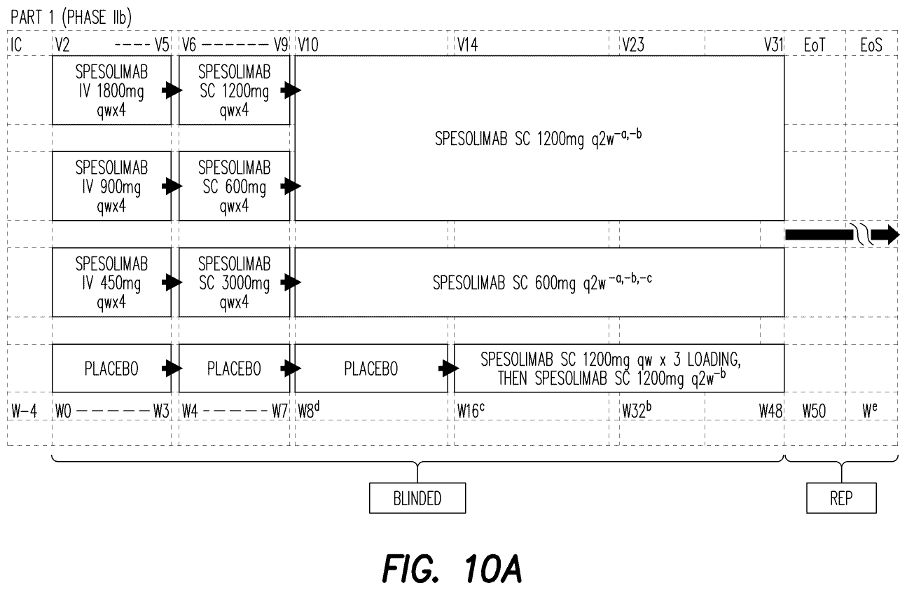

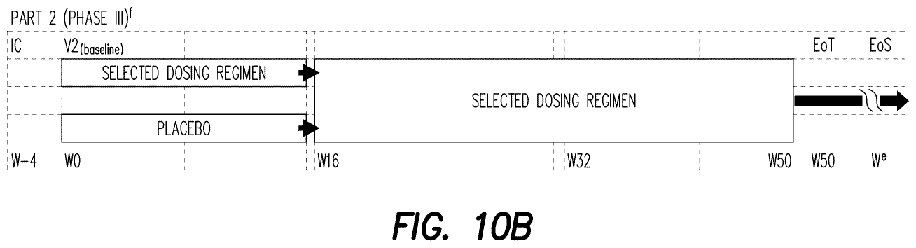

There was a sustained reduction in all lesion counts. Sustained improvements in HS severity and patient-reported outcomes were observed. Clinical benefits with spesolimab were also observed in patients switching from placebo (before the OLE) to spesolimab. FIG. 9A-9D shows the absolute change from baseline to Week 50 for lesion types and IHS4 score: Patient numbers at baseline were prior spesolimab n=30 and prior placebo n=15. For change from baseline, only patients with ≥1 relevant lesion type were included. Patient numbers at Week 50 were prior spesolimab n=15 and prior placebo n=7. FIG. 9A. demonstrates the mean (95% CI) absolute change from baseline in draining tunnel count. Prior spesolimab group=−1.3 (−2.9, 0.3); Prior placebo group=−3.7 (0.3, −7.8) (Only patients with ≥1 draining tunnel at baseline are included). FIG. 9B. demonstrates the mean (95% CI) absolute change from baseline in inflammatory nodule count. Prior spesolimab group=−3.7 (−7.9, 0.5); Prior placebo group=−5.7 (−8.7, −2.8) (Only patients with ≥1 inflammatory nodule at baseline are included). FIG. 9C. demonstrates the mean (95% CI) absolute change from baseline in abscess count. Prior spesolimab group=−0.9 (−2.2, 0.5); Prior placebo group=−2.4 (−7.6, 2.8) (Only patients with ≥1 abscess at baseline are included). FIG. 9D. demonstrates the Mean (95% CI) absolute change from baseline in IHS4 score. Prior spesolimab group=−12.8 (−20.7, −5.0); Prior placebo group=−23.4 (−50.8, 4.0).

Improvements in secondary and further efficacy endpoints were sustained through Week 50 are shown in Table 17 below.

TABLE 17
Absolute change from baseline to Week
50 in secondary and further endpoints

| | | |
|---|---|---|
| | Prior spesolimab | Prior placebo |
| Endpoint | (n = 15) | (n = 7) |

Absolute change from baseline at Week 50, mean (95% CI)

| | | | | |
|---|---|---|---|---|
| Total abscess, inflammatory | −7.9 | (−11.6, −4.3) | −9.9 | (−22.0, 2.3) |
| nodule, and draining tunnel | | | | |
| count | | | | |
| HASI | −36.8 | (−48.8, −24.8) | −22.1 | (−49.5, 5.2) |
| DLQI | −5.4 | (−10.0, −0.7) | −4.4 | (−8.6, −0.3) |
| HiS-QOL | −13.1 | (−24.0, −2.2) | −7.0 | (−16.3, 2.3) |

Patients achieving endpoints at Week 50, n/N (%)

| | | | | |
|---|---|---|---|---|
| HiSCR^{†} | 10/15 | (66.7) | 5/6 | (83.3) |
| IHS4-55 response | 10/15 | (66.7) | 6/7 | (85.7) |
| Complete elimination of | 7/13 | (53.8) | 4/6 | (66.7) |
| draining tunnels^{†} | | | | |
| Complete elimination of | 7/9 | (77.8) | 3/3 | (100.0) |
| abscesses^{‡} | | | | |

n numbers are for Week 50; at baseline patient numbers were prior spesolimab n = 30 and prior placebo n = 15.
^{†}Only patients with ≥1 draining tunnel at baseline are included.
^{‡}Only patients with ≥1 abscess at baseline are included.
^{§}Only patients with a value of ≥1 on the NRS of Patient's Global Assessment of HS pain were included. CI, confidence interval; DLQI, Dermatology Life Quality Index; HASI, Hidradenitis Suppurativa Area and Severity Index; HiSCR, Hidradenitis Suppurativa Clinical Response; HiS-QOL, Hidradenitis Suppurativa Quality of Life; HS, hidradenitis suppurativa; IHS4, International Hidradenitis Suppurativa Severity Score System; NRS, numerical rating scale; OLE, open-label extension.

### Example 2: Phase IIb-III Clinical Trial: Treating Patients with Moderate-Severe Hidradenitis Suppurativa

The following example is a follow-on of the study described in Example 1.

Objectives: Study 1368-0098 consists of 2 parts: dose finding Phase IIb (Part 1), designed to find the optimal dose in patients with moderate to severe HS for Phase III development; confirmatory Phase III (Part 2), designed to assess the efficacy and safety of spesolimab in patients with moderate to severe HS. The dose finding phase (Part 1) will characterise the dose-response curve and exposure-response relationship and will support identifying the dose of spesolimab providing the most adequate benefit/risk to patients with moderate to severe HS by assessing dosing regimens.

The primary objective of dose finding phase are as follows:
(1) to demonstrate a non-flat dose response curve, evaluate the quantitative treatment effect size, and evaluate the dose-response relationship based on the primary endpoint of percent change from baseline in dT count at Week 8
(2) to determine an optimal dose candidate for Part 2 by incorporating information of available safety and of efficacy based on the primary endpoint of percent change from baseline in dT count at Week 8

### The Secondary Objectives of Dose Finding Phase are as Follows:

(1) to demonstrate non-flat dose response curve based on selected secondary and further efficacy endpoints at Week 8.
(2) to evaluate the quantitative treatment effect size of percent change from baseline in dT count at Week 16 and absolute change from baseline in International Hidradenitis Suppurativa Severity Score System (IHS4) value at Week 16.
(3) to evaluate the sustainability of spesolimab efficacy up to Week 24 and up to Week 50.
(4) to assess safety of spesolimab.

### The Objectives of the Confirmatory Phase are as Follows:

The confirmatory phase is designed to provide substantial evidence of the efficacy, safety, and tolerability of spesolimab in comparison to placebo in trial participants with moderate to severe HS.

The primary objective of the confirmatory phase (Part 2) is to demonstrate superiority of one dosing regimen (loading and maintenance dose for each) of spesolimab (identified on the basis of Phase IIb data analysis) against placebo in the mean percent change from baseline in dT count at Week 16.

The dose (loading dose and maintenance dose) for Part 2 (Phase III) is based on primary analysis when the last trial participant completes Visit 10 (Week 8).

### The Secondary Objectives are as Follows:

(1) to demonstrate superiority of spesolimab against placebo for the key secondary endpoints
(2) to assess safety of spesolimab.

### Overall Design:

The international, Phase IIb/III multi-center, double-blind, placebo-controlled, randomised trial assesses the efficacy and safety of spesolimab versus placebo in patients with moderate to severe HS. The primary endpoint, percent change from baseline in dT count at Week 8, and the secondary endpoint, percent change from baseline in dT count at Week 16, were chosen due to the positive correlation observed between drug exposure and response (reduction in dT count) at the dose regimens tested in trials 1368-0052 and 1368-0067 (Example 1). Draining tunnels are lesions with high impact for the trial participants, and drainage represents one of the highest unmet needs. The pathophysiology of dT suggests a potential key role for the blockade of IL36 receptor activation by spesolimab. Based on the efficacy results in trial 1368-0052, clinical outcome as early as Week 8 are sufficient to evaluate the dose-exposure-response relationship for efficacy.

The absolute change from baseline in IHS4, HASI, and HiSCR values at Week 8, have been chosen to reinforce dT count at Week 8 (the primary endpoint for analysis).

Data from prior studies of spesolimab in HS are indicative of a need for dose ranging work and for further investigations over the efficacy profile of this molecule in HS. The primary analysis is conducted after all trial participants complete Week 8 visit. In addition, a Week 16-interim analysis is conducted after all trial participants complete Week 16 visit. These two pre-specified analyses are determinative of the selection of the dosing regimen for Part 2 as well as aiding any necessary adjustment to the primary and key secondary endpoints, sample size, and trial participant population. It is understood that based on modelling results, the loading/maintenance dosing regimen in Part 2 may differ from the dosing regimens tested up to Week 8 in Part 1, and therefore adjusted accordingly. For example, dosing amount and frequency can be adjusted based on observed responsiveness and the defined primary and secondary endpoint measures (e.g, the potential benefit of the highest dosing regimen will be determined with up to Week 8 data, limiting unnecessary over exposure until Week 16 if no benefit is shown; and lower dose group trial participants may be up-titrated to a higher dose based on clinical response, as per protocol).

Approximately 200 trial participants in Part 1 (Phase IIb) and 260 trial participants in Part 2 (Phase III) are randomised. An overview of trial design is presented in FIGS. 9A & 9B.

After signing the informed consent, trial participants are screened for a period for up to 28 days, and if all eligibility criteria are met, trial participants are randomised in a 1:1:1:1 ratio to either active group, including high dose, medium dose, and low dose group, or placebo group (50 trial participants in each group). The randomisation is stratified for TNFi status (TNFi-naive population versus TNFi-exposed population) and dT count at baseline (dT count≤3; 4-10; >10).

At least approximately 60 trial participants from TNFi-exposed population (meaning a corresponding 140 participants being TNFi-naïve) and a maximum of approximately 100 trial participants from TNFi-exposed population (meaning a corresponding 100 participants being TNFi-naïve) are randomised, respectively. These participants who were previously exposed to TNFi might have had a primary or a secondary failure of TNFi treatment. Primary failure of TNF treatment is defined as lack of efficacy after at least 3 months of treatment with an agent blocking TNF-alpha (patients who never responded), and secondary failure is defined as loss of clinical efficacy in a trial participant after initial response to an agent blocking TNF-alpha (patients who initially responded but then relapsed). Additionally, TNFi-exposed population might have stopped TNFi due to AE or stopped for other reason.

Approximately 100 trial participants with baseline dT count≤3, and 100 trial participants with baseline dT count>3 are planned to be randomised, respectively.

### Treatment Phase

Once randomised, trial participants start a treatment period of 50 weeks. Administration of treatment will be up to Week 48. The final assessment of treatment effect will be on Week 50 (EoT visit).

For the first four weeks, according to the assigned dose group, trial participants will be administered an initial loading i.v. dose of spesolimab (either 1800 mg [high dose group], 900 mg [medium dose group], or 450 mg [low dose group] every week at Visit 2 [Week 0], Visit 3 [Week 1], Visit 4 [Week 2], and Visit 5 [Week 3]) or matching placebo.

From Visit 6 (Week 4) to Visit 9 (Week 7) (both inclusive), trial participants will be administered a weekly maintenance s.c. dose of spesolimab (either 1200 mg [high dose group], 600 mg [medium dose group], or 300 mg [low dose group] according to the assigned dose group), or matching placebo. For trial participants initially randomised to spesolimab group, from Visit 10 (Week 8) to Visit 31 (Week 48) (both inclusive), the participants will be administered a maintenance s.c. dose of spesolimab: either 1200 mg (high and medium dose group) or 600 mg (low dose group) s.c. every 2 weeks according to the assigned dose group).

From Visit 14 (Week 16), there is an option of increasing the dose to the maximum subcutaneous dose of 1200 mg every two weeks (applied to low dose treatment group participants only, via IRT in a blinded manner) if they have inadequate clinical response defined as 25% increase in the ANdT count compared to baseline and the investigator agrees with the dose increase.

For trial participants initially randomised to placebo group, from Visit 10 (Week 8) to Visit 13 (Week 14) (both inclusive), the participants will be administered a matching placebo every 2 weeks; from Visit 14 (Week 16) to Visit 31 (Week 48) (both inclusive), the participants will be switched to a s.c. dose of spesolimab (starting with 3 loading s.c. doses of spesolimab 1200 mg every week and then maintenance s.c. dose of spesolimab 1200 mg every 2 weeks).

From Visit 23 (Week 32) on, trial participants will continue to receive the assigned dosing regimen or adjusted according to a primary analysis at Week 8 whereupon the dosing regimen may be adapted from Visit 23 (Week 32).

Trial participants completing EoT visit (Week 50) of the study may roll over to an OLE study if they agree and meet the eligibility criteria of the OLE trial. These trial participants are not required to complete a safety follow-up period, and their EoT visit will be considered also their end of study (EoS) visit, which is the first visit in the OLE trial.

Trial participants, who permanently discontinue trial drug earlier than Visit 31 (Week 48), or do not qualify to enter the OLE trial for any other reason, will be invited to do the EoT visit instead of the next planned visit and will then enter a safety follow-up period (16 weeks after the last drug administration).

### Inclusion Criteria:

### Part 1 (Phase IIb)

Of full age of consent (according to local legislation, at least ≥18 years) at screening.

Signed and dated written informed consent in accordance with ICH-GCP and local legislation prior to admission to the trial.

Moderate to severe HS, based on IHS4 criteria, at least 6 months prior to and including Baseline visit, as determined by the investigator through participant interview and/or review of the medical history (if IHS4 scoring is not available for the period before screening, equivalent scoring based on scoring systems as HS-PGA or Hurley are acceptable on the basis of documented investigator assessment)

HS lesions in at least 2 distinct anatomic areas (right/left axilla, right/left inguino-crural fold, right/left inframammary fold, intermammary zone, right/left buttock, perineal, other)

Biologic naive or TNFi-exposed for HS.

For biologic naïve, inadequate response to an adequate course of appropriate oral antibiotics for treatment of HS in the last 1 year prior to the Baseline visit, as per investigator discretion.

Total AN count of greater than or equal to 5 at Baseline visit.

Total dT count of at least 1 at Baseline visit.

Women of childbearing potential (WOCBP) must be ready and able to use highly effective methods of birth control per ICH M3 (R2) that result in a low failure rate of less than 1% per year when used consistently and correctly, for the duration of the trial and 16 weeks after last administration.

### Part 2 (Phase III)

Except for potential criteria, which will be confirmed after primary analysis of Phase IIb results, the same inclusion criteria as Part 1 (Phase IIb) will need to be met.

### Exclusion Criteria:

### Part 1 (Phase IIb)

a. Participants who must or wish to continue the intake of restricted medications or any drug considered likely to interfere with the safe conduct of the trial.
b. Prior exposure to any immunosuppressive biologic other than TNFi for HS.
c. Prior exposure to IL-36R inhibitors including spesolimab.
d. Treated with any investigational device or investigational drug of chemical or biologic nature within a minimum of 30 days or 5 half-lives of the drug, whichever is longer, prior to Visit 2 (Baseline visit)
e. Women who are pregnant, nursing, or who plan to become pregnant while in the trial. Women who stop nursing before the study drug administration do not need to be excluded from participating.
f. Participants with history of allergy/hypersensitivity to the systemically administered trial medication agent or its excipients.
g. Participants with a transplanted organ (with exception of a corneal transplant>12 weeks prior to screening) or who has ever received stem cell therapy (e.g., Remestemcel-L)
h. Participants with any documented active or suspected malignancy or history of malignancy within 5 years prior to the screening visit, except appropriately treated basal cell carcinoma of the skin, squamous cell carcinoma of the skin, or in situ carcinoma of uterine cervix.
i. Participants with active or latent tuberculosis (TB) should be excluded.
j. Participants with active systemic infection within 2 weeks of Visit 2 (Baseline visit). These participants can be re-screened after completed treatment of the acute infection, as per investigator discretion.
k. Participants with relevant chronic infections as determined by the investigator, including human immunodeficiency virus (HIV) or viral hepatitis. The corresponding laboratory tests will be performed during screening. In case of a positive hepatitis C antibody test, a positive reflex testing for Hepatitis C RNA PCR is considered positive. A patient can be re-screened if the patient was treated and is cured from the acute infection.
l. Major surgery (major according to the investigator's assessment) performed within 12 weeks prior to first study drug administration (Visit 2-Baseline visit) or planned during the study (e.g., hip replacement, aneurysm removal, stomach ligation)
m. Participants with severe, progressive, or uncontrolled hepatic disease, defined as >3-fold Upper Limit of Normal (ULN) elevation in AST, ALT, or alkaline phosphatase, or >2-fold ULN elevation in total bilirubin at screening visit and/or Visit 2 (Baseline visit). Trial participants with Gilbert's syndrome can be included unless total bilirubin elevation was >5-fold ULN at screening visit and unless proportions of bilirubin fractions are inconsistent with diagnosis of Gilbert's syndrome.
n. Participants at screening with a severe, progressive, or uncontrolled condition as renal, hepatic, haematological, endocrine, pulmonary, cardiac, neurologic, cerebral, psychiatric disease (including history or suspicion of chronic alcohol or drug abuse) or other than HS, or signs and symptoms thereof, in the opinion of the investigator would compromise the safety of the participant or the quality of the data, and would make the study participant unreliable to adhere to the protocol, to comply with all study visits/procedures, or to complete the trial.
o. Planned use of laser or other hair removal procedures over HS-affected areas during the trial period.
p. Participants with any suicidal ideation of type 4 or 5 on the C-SSRS in the past 12 months prior to screening visit (i.e., active suicidal thoughts with method and intent but without specific plan, or active suicidal thoughts with method, intent and plan)
q. Participants with any suicidal behavior in the past 2 years prior to screening visit (i.e., actual attempt, interrupted attempt, aborted attempt, or preparatory acts or behavior)
r. Previous enrolment in this trial (exception: trial participants re-screened)
s. Presence of acute demyelinating polyneuropathy.
t. Confirmed or suspected acute SARS COV 2 infection. Participants who recovered from a SARS COV 2 infection may be eligible: 14 days or more from first symptoms or from first PCR test confirmation to randomisation, whichever is shorter AND at least 14 days without SARS COV 2 symptoms. If local criteria are stricter, they will apply.

### Part 2 (Phase III)

Except for potential criteria, which will be confirmed after analysis of Phase IIb results, the same criteria as Part 1 (Phase IIb) will need to be met.

### Relevant Trial Activities: See Flowchart: Part 1 (Phase IIb) (FIGS. 11A & 11B)

As illustrated in FIGS. 11A & 11B, visits are labeled correspondent to the weeks and the ideal time window for the visit. Relevant activities are further described below as indicated as footnotes in the flowchart (FIGS. 11A & 11B).

For Visits 7, 9, 15, 20, 22, 24, 26, 28, and 30, trial participants will be offered the possibility to have home nursing visits if locally allowed. In exceptional circumstances (i.e., pandemic, conflict, other), visits might take place by phone just to collect safety information. (FN1). Trial participants who discontinue trial treatment prematurely undergo the End of Treatment (EoT) visit as soon as possible. All trial participants not entering the open label extension (OLE) trial are expected to complete EoT visit, and EoS visit 16 weeks after their last trial drug intake or 14 weeks from EoT. For trial participants entering the OLE trial at Week 50, the EoT visit will be their EOS visit in trial 1368-0098 (FN 2, also see FN 10 and FN 20).

X^{c} indicates the time when a complete physical examination is given. X^{t} is indicates when a targeted physical examination is given (FN 3). Measurements of vital signs should precede blood sampling to avoid the impact of blood sampling on the vital measurements at all dosing visits. Additional assessments of vital signs should be performed approximately at 10 minutes post-dose for s.c. administrations and approximately 5 minutes and 1 hour post dose for i.v. administrations (FN 4).

Women of childbearing potential are given a serum pregnancy test performed at screening. Urine pregnancy tests will be performed at all other visits indicated in the Flowchart (FIGS. 11A & 11B) (FN 5).

It is preferred, but trial participants do not have to be fasted for the blood sampling for the safety laboratory. At visits with study drug administration, this should be done prior to the study drug administration (FN6).

Infection testing is done at screening and at EoT visit (FN7).

ECG measurements precede blood sampling and drug administration. The ECG measurements are conducted at timepoints indicated in the Flowchart (FIGS. 11A & 11B). Additional ECG measurements may be performed if the investigator deems it clinically indicated. (FN 8).

At study visits with study drug administration, pre-dose PK/ADA/NAb samples will be obtained approximately within 2 hours prior to start of i.v. infusion or s.c. injection. At Week 0/Day 1, post-dose PK samples are obtained approximately 5 mins after end of i.v. infusion (FN 9). Trial participants who terminate study drug early should do the EoT visit as soon as possible. These participants will then do EoS visits, 16 weeks after last study drug administration (FN 10).

Desoxyribonucleic Acid (DNA) banking sample is optional. This sampling is only possible if the trial participant agreed by signing a separate informed consent (FN 11). Skin biopsy is mandatory at selected sites and should be sampled under ultrasound (US) guidance. For all other sites it is optional (FN12). US lesion evaluation is mandatory at selected sites with US capabilities. For all other sites it is optional (FN13).

IHS4 is used to classify trial participant's severity at baseline. IHS4 will be derived from lesions count by a trained health care professional (FN 14). Trial participant diaries are given to trial participants with the objective to collect daily information about NRS Pain and Pruritus from baseline, till Visit 19 (Week 24). After Visit 19, NRS pain and NRS pruritus will be collected on a daily basis during the 7 days prior to the next scheduled visit (prior to Visit 23, Visit 27, Visit 31, and EoT visit). Site staff will collect the diaries from trial participants at visits indicated in the Flowchart (FIGS. 11A & 11B) to assess pain and pruritus since last visit (FN 15).

At Screening, the C-SSRS baseline/screening scale will be completed. At all subsequent visits the “since last visit” scale will be completed. (FN 16).

At Visit 2, instructions for paper version of patient's daily diary will be dispensed to trial participants. Site staff should provide adequate training. Two weekly diaries and one additional weekly diary to capture surplus time window of visit will be dispensed to trial participants. The diary will contain two scales, one for pruritus and one for pain. Trial participant's diary will capture daily NRS pruritus and pain score (and analgesic use, and other interventions to manage pain) until Visit 19. From Visit 23, trial participants will record data for pain and pruritus daily on the last 7 days before the next scheduled visit (FN 17a). Once electronic diary is available instructions for electronic diary will be dispensed to trial participants. Trial participant diary captures daily NRS score in two scales, one for pruritus and another for pain until Visit 19. From Visit 23, trial participants will record data for pain and pruritus during the last 7 days before the applicable visits (one daily diary on the last 7 days before the next scheduled visit). Analgesic use and other interventions to manage pain will be collected at the same time points, but in paper format (FN 17b). Trial participant's diaries returned by participants must be reviewed while the trial participant is in the consultation, so that any information can be clarified in an interview with the trial participant, if needed (FN 18).

For local tolerability at the administration site of the IMP, the investigator will perform an assessment during the study drug administration visit and at the time of AE assessment by questioning retrospectively since the last visit. Any observed local tolerability reaction, e.g., “swelling”, “induration”, “heat”, “redness” will be evaluated and reported as adverse events (FN 19).

All trial participants completing Week 50 of the trial may be offered to enter the OLE trial. For these participants, the EoT visit will coincide with EoS visit for Part 1 of trial 1368-0098, and the first visit of the OLE trial. (FN 20)

### Primary Outcome Measures:

Part 1 (Phase IIb): Percent change from baseline in dT count at Week 8.

Part 2 (Phase III): Percent change from baseline in dT count at Week 16.

### Secondary Outcome Measures:

### Part I (Phase IIb:)

a. Percent change from baseline in dT count at Week 16.
b. Absolute change from baseline in IHS4 value at Week 8.
c. Absolute change from baseline in IHS4 value at Week 16.

### Part II (Phase III):

a. Absolute change from baseline in IHS4 value at Week 16.
b. Absolute change from baseline in Hidradenitis Suppurativa Area Severity Index (HASI) score at Week 16.
c. Achievement of Hidradenitis Suppurativa Clinical Response (HiSCR50) at Week 16:
d. HiSCR50 is defined as at least a 50% reduction in the total abscess and inflammatory nodule (AN) count with no increase in abscess count and no increase in dT count relative to baseline.
e. Percent change from baseline in abscess count at Week 16.
f. Achievement of at least a 50% reduction in dT count at Week 16 relative to baseline.
g. Achievement of at least a 50% reduction in abscess and dT (AdT) count at Week 16 relative to baseline
h. Achievement of at least a 50% reduction in ANdT count at Week 16 relative to baseline.
i. Achievement of at least 30% reduction from baseline in Numerical rating Scale (NRS30) in Patient's Global Assessment of HS pain at Week 16.

### Further Endpoints:

### Part I (Phase IIb) Efficacy:

### Lesion Counts Clinical Endpoints

a. Percent change from baseline in dT count at each scheduled assessment.
b. Achievement of at least 50% reduction from baseline in dT count at each scheduled assessment.
c. Occurrence of complete elimination of dT at each scheduled assessment.
d. Time to first occurrence of complete elimination of dT during the treatment period.
e. Percent change from baseline in abscess count at each scheduled assessment.
f. Achievement of at least 50% reduction from baseline in abscess count at each scheduled assessment.
g. Occurrence of complete elimination of abscess at each scheduled assessment.
h. Percent change from baseline in total AdT count at each scheduled assessment.
i. Achievement of at least 50% reduction from baseline in AdT count at each scheduled assessment.
j. Percent change from baseline in inflammatory nodule (N) count at each scheduled assessment.
k. Percent change from baseline in total ANdT count at each scheduled assessment.
l. Achievement of at least 50% reduction from baseline in ANdT count at each scheduled assessment.

### Response-, Score-, and Flare-Related Clinical Endpoints

a. Achievement of HiSCR50 at each scheduled assessment.
b. Absolute change from baseline in IHS4 value at each scheduled assessment.
c. Achievement of IHS4-55 response (defined as at least 55% reduction from baseline) at each scheduled assessment.
d. Absolute change from baseline in HASI score at each scheduled assessment.
e. Achievement of Physician's Global Assessment (PGA) score of 0 or 1 at each scheduled assessment.
f. Occurrence of at least one HS flare at each scheduled assessment: defined as at least 25% increase in AN count with a minimum increase of 2 relative to baseline.
g. Time to first occurrence of HS flare during the treatment period.

### Patient Reported Outcomes (PROs)

a. Achievement of at least 30% reduction from baseline in Numerical rating scale (NRS30) in Patient's Global Assessment of HS pain at each scheduled assessment.
b. Absolute change from baseline in Hidradenitis Suppurativa Quality of Life (HIS-QoL) total score at each scheduled assessment.
c. Absolute change from baseline in FACIT-Fatigue scale score at each scheduled assessment.
d. Absolute change from baseline in Dermatology Life Quality Index (DLQI) score at each scheduled assessment.
e. Absolute change from baseline in Patient Global Impression of Change (PGI-C) score over time.
f. Absolute change from baseline in Patient Global Impression of Severity (PGI-S) score over time.
g. Change from baseline in Hidradenitis Suppurativa odour and drainage scale (HODS) at each scheduled assessment.
h. Change from baseline in NRS Pruritus at each scheduled assessment.
i. Change from baseline in Hospital Anxiety and Depression Scale (HADS) at each scheduled assessment.

### Part 2 (Phase III): Efficacy

a. Achievement of HISCR50 at each scheduled assessment.
b. Percent change from baseline in dT count at each scheduled assessment.
c. Achievement of at least 50% reduction from baseline in dT count at each scheduled assessment.
d. Occurrence of complete elimination of dT at each scheduled assessment.
e. Time to first occurrence of complete elimination of dT during the treatment period.
f. Percent change from baseline in abscess count at each scheduled assessment.
g. Achievement of at least 50% reduction from baseline in abscess count at each scheduled assessment.
h. Occurrence of complete elimination of abscess at each scheduled assessment.
i. Percent change from baseline in total AdT count at each scheduled assessment.
j. Achievement of at least 50% reduction from baseline in AdT count at each scheduled assessment.
k. Percent change from baseline in N count at each scheduled assessment.
l. Absolute change from baseline in IHS4 value at each scheduled assessment.
m. Achievement of IHS4-55 response (defined as at least 55% reduction from baseline) at each scheduled assessment.
n. Absolute change from baseline in HASI score at each scheduled assessment.
o. Achievement of at least 30% reduction from baseline in Numerical rating scale (NRS30) in Patient's Global Assessment of HS pain at each scheduled assessment.
p. Percent change from baseline in total ANdT count at each scheduled assessment.
q. Achievement of at least 50% reduction from baseline in ANdT count at each scheduled assessment.
r. Achievement of PGA score of 0 or 1 at each scheduled assessment.
s. Absolute change from baseline in HIS-Qol total score at each scheduled assessment.
t. Absolute change from baseline in FACIT-Fatigue scale score at each scheduled assessment.
u. Absolute change from baseline in DLQI score at each scheduled assessment.
v. Absolute change from baseline in PGI-C score over time.
w. Absolute change from baseline in PGI-S score over time.
x. Change from baseline in HODS at each scheduled assessment.
y. Change from baseline in NRS Pruritus at each scheduled assessment.
z. Change from baseline in HADS at each scheduled assessment.
aa. Occurrence of at least one HS flare at each scheduled assessment: defined as at least 25% increase in AN count with a minimum increase of 2 relative to baseline.
bb. Time to first occurrence of HS flare during the treatment period.
cc. Work Productivity and Activity Impairment Questionnaire for HS (WPAI-HS)—change from baseline in WPAI-HS at each scheduled assessment.
dd. EQ-5D-5L-change from baseline in EQ-5D at each scheduled assessment.
ee. Number of surgeries
ff. Days of hospitalisation
gg. Days out of work/unemployment
hh. Number of emergency room visits
ii. Steroid free time/remission
jj. Hormonal therapy free time (in women)
kk. Antibiotics free time/remission

### Adverse Events; See Discussion in Example I Above.

### Treatments Administered

TABLE 18
Test product spesolimab solution for infusion

| | |
|---|---|
| Substance: | Spesolimab (Spevigo ®) |
| Pharmaceutical formulation: | Solution for infusion |
| Unit Strengths: | Spesolimab 450 mg/vial (concentration: |
| | 60 mg/mL, fill volume: 7.5 mL) |
| Route of Administration: | intravenous infusion |
| Posology: | Over 90 minutes. It can be adapted if |
| | needed. (The total duration should not |
| | exceed 180 minutes (3 hours). |

TABLE 19
Test product matching placebo solution for infusion

| | |
|---|---|
| Substance: | Placebo matching to spesolimab |
| Pharmaceutical formulation: | Solution for infusion |
| Unit Strengths: | Vials with fill volume: 7.5 mL |
| Route of Administration: | intravenous infusion |
| Posology: | Over 90 minutes. It can be adapted if |
| | needed. (The total duration should not |
| | exceed 180 minutes (3 hours). |

TABLE 20
Test product spesolimab solution for injection

| | |
|---|---|
| Substance: | Spesolimab (Spevigo ®) |
| Pharmaceutical | Solution for injection |
| formulation: | |
| Unit Strengths: | Spesolimab 300 mg/pre-filled syringe |
| | (concentration: 150 mg/mL, fill volume: 2 mL) |
| Route of Administration: | Subcutaneous injection |
| Posology: | See Tables 19-22 below. |

TABLE 21
Test product matching placebo solution for injection

| | |
|---|---|
| Substance: | Placebo matching to spesolimab |
| Pharmaceutical formulation: | Solution for injection |
| Unit Strengths: | Vials with a fill volume of 2 mL |
| Route of Administration: | Subcutaneous injection |
| Posology: | See Tables 19-22 below. |

TABLE 22
Placebo-controlled Dosing Treatment Period Part I (Up to Visit 14 (week 16))
Administration of trial drug

| Visit | | | | | | | | | | | | | |
|---|---|---|---|---|---|---|---|---|---|---|---|---|---|
| 1 | 2 | 3 | 4 | 5 | 6 | 7 | 8 | 9 | 10 | 11 | 12 | 13 | 14 |

Week

| | | | | | | | | | | | | |
|---|---|---|---|---|---|---|---|---|---|---|---|---|
| Wk 0 | Wk 1 | Wk 2 | Wk 3 | Wk 4 | Wk 5 | Wk 6 | Wk 7 | Wk 8 | Wk 10 | Wk 12 | Wk 14 | Wk 16 |

| | | |
|---|---|---|
| | i.v. Loading Dose | s.c. Maintenance Dose |
| Wk −5 | (in mg) | (in mg) |

| | | | | | | | | | | | | | |
|---|---|---|---|---|---|---|---|---|---|---|---|---|---|
| Arm 1 | 1800 | 1800 | 1800 | 1800 | 1200 | 1200 | 1200 | 1200 | 1200 | 1200 | 1200 | 1200 | 1200 |
| Arm 2 | 900 | 900 | 900 | 900 | 600 | 600 | 600 | 600 | 1200 | 1200 | 1200 | 1200 | 1200 |
| Arm 3 | 450 | 450 | 450 | 450 | 300 | 300 | 300 | 300 | 600 | 600 | 600 | 600 | 600 |
| Arm 4 | i.v. P | i.v. P | i.v. P | i.v. P | s.c. P | s.c. P | s.c. P | s.c. P | s.c. P | s.c. P | s.c. P | s.c. P | 1200 |

Wk = Week (study weeks),
i.v. = intravenous,
s.c. = subcutaneous,
P = Placebo

### Part 1 (Phase IIb)

Spesolimab Test Groups up to Visit 14 (Week 16):
a. High dose group (1800 mg i.v. qw×4, 1200 mg s.c. qw×4 [up to Visit 9 (Week 7) included], then 1200 mg s.c. q2w until Visit 14 [Week 16])
b. Medium dose group (900 mg i.v. qw×4, 600 mg s.c. qw×4 [up to Visit 9 (Week 7) included], then 1200 mg s.c. q2w until Visit 14 [Week 16])
c. Low dose group (450 mg i.v. qw×4, 300 mg s.c. qw×4 [up to Visit 10 (Week 8) included], then 600 mg s.c. q2w until Visit 14 [Week 16])

### Placebo Group:

a. Up to Visit 14 (Week 16) but not including this visit, matching.
b. From Visit 14 (Week 16), trial participants in the placebo group will be switched to s.c. 1200 mg q2w until Visit 23 (Week 32), starting with a loading dose of spesolimab 3600 mg s.c. (1200 mg s.c. qw×3)
c. From Visit 23 (Week 32) on, trial participants will continue to receive the previously assigned dosing regimen until the results of primary analysis (Week 8) are available. Upon availability of the Phase IIb primary analysis efficacy and safety results, the dosing regimen might be adapted from Visit 23 (Week 32) through amendment.

Administration of treatment will be up to Week 48. The final assessment of treatment effect will be on Week 50 (EoT visit).

TABLE 23
Overview of treatment course from Visit 15 (week 17) up to Visit 31 (week 48)
Administration of trial drug

| | Visit | | | | | | | | | | | | | | | | |
|---|---|---|---|---|---|---|---|---|---|---|---|---|---|---|---|---|---|
| | 15 | 16 | 17 | 18 | 19 | 20 | 21 | 22 | 23 | 24 | 25 | 26 | 27 | 28 | 29 | 30 | 31 |
| | Week | | | | | | | | | | | | | | | | |
| | Wk | Wk | Wk | Wk | Wk | Wk | Wk | Wk | Wk | Wk | Wk | Wk | Wk | Wk | Wk | Wk | Wk |
| | 17 | 18 | 20 | 22 | 24 | 26 | 28 | 30 | 32 | 34 | 36 | 38 | 40 | 42 | 44 | 46 | 48 |
| | s.c. Maintenance Dose (in mg) | | | | | | | | | | | | | | | | |
| Arm 1 | s.c. P | 1200 | 1200 | 1200 | 1200 | 1200 | 1200 | 1200 | 1200 | 1200 | 1200 | 1200 | 1200 | 1200 | 1200 | 1200 | 1200 |
| Arm 2 | s.c. P | 1200 | 1200 | 1200 | 1200 | 1200 | 1200 | 1200 | 1200 | 1200 | 1200 | 1200 | 1200 | 1200 | 1200 | 1200 | 1200 |
| Arm 3 | s.c. P | 600 | 600 | 600 | 600 | 600 | 600 | 600 | 600 | 600 | 600 | 600 | 600 | 600 | 600 | 600 | 600 |
| Arm 4 | 1200 | 1200 | 1200 | 1200 | 1200 | 1200 | 1200 | 1200 | 1200 | 1200 | 1200 | 1200 | 1200 | 1200 | 1200 | 1200 | 1200 |

Wk = Week (study weeks),
s.c. = subcutaneous,
P = Placebo

Spesolimab Test Group from Visit 14 (Week 16)-Visit 23 (Week 32):
a. From Visit 14 (Week 16), trial participants in high and medium dose groups will receive 1200 mg s.c. q2w until Visit 23 (Week 32).
b. From Visit 14 (Week 16), trial participants in low dose group will receive 600 mg s.c. q2w until Visit 23 (Week 32).
c. Based on inadequate clinical response as per protocol, up-titration from 600 mg q2w to 1200 mg q2w will be possible when applicable (low dose group).

From Visit 23 (Week 32) on, trial participants continue to receive the previously assigned dosing regimen until the primary analysis (Week 8). Based on the primary analysis efficacy and safety results, the dosing regimen may be adapted from Visit 23 (Week 32).

### Placebo:

a. Up to Week 16, matching. From Week 16, trial participants in the placebo group will be switched to a dose regimen selected on the basis of Phase IIb.

Administration of treatment will be up to Week 50. The final assessment of treatment effect will be on Week 52 (EoT visit).

TABLE 24
Dosage and treatment schedule for the intravenous loading phase

| | Visit 2 (baseline) | Visit 3 | Visit 4 | Visit 5 |
|---|---|---|---|---|
| | Week 0 | Week 1 | Week 2 | Week 3 |
| | Number of 450 mg spesolimab vials with a fill volume | | | |
| | of 7.5 mL solution for infusion/matching placebo | | | |
| Arm 1 | 4/0 | 4/0 | 4/0 | 4/0 |
| Arm 2 | 2/2 | 2/2 | 2/2 | 2/2 |
| Arm 3 | 1/3 | 1/3 | 1/3 | 1/3 |
| Arm 4 | 0/4 | 0/4 | 0/4 | 0/4 |

TABLE 25
Dosage and treatment schedule for subcutaneous phase

| | Visits | Visits | Visits | | Visits |
|---|---|---|---|---|---|
| | 6 to 9 | 10 to 13 | 14 and 16 | | 17 to 23 |
| | Weeks | Weeks | Weeks | Visit 15 | Weeks |
| | 4 to 7* | 8 to 14** | 16 and 18* | Week 17* | 20 to 32** |
| | Number of 300 mg spesolimab Pre-Filled | | | | |
| | Syringes (PFS) with a fill volume of 2 mL | | | | |
| | (concentration: 150 mg/mL) for s.c. injection/matching placebo | | | | |
| Arm 1 | 4/0 | 4/0 | 4/0 | 0/4 | 4/0 |
| Arm 2 | 2/2 | 4/0 | 4/0 | 0/4 | 4/0 |
| Arm 3 | 1/3 | 2/2 | 2/2 | 0/4 | 2/2 |
| Arm 4 | 0/4 | 0/4 | 4/0 | 4/0 | 4/0 |

*qw
**q2w

### Results

Outcomes of efficacy derived from HS lesions count include Part 1 and Part 2 proposed primary endpoint; percent change from baseline in dT count. Tunnel/fistula/sinus should be counted as dT only if draining. Other outcomes of efficacy derived from lesions count are the % change from baseline for each type of lesions or their combination, IHS4, and HiSCR50. The HASI is derived from a detailed clinical assessment of the extent and intensity of HS lesions according to specific criteria.

The HS-PGA is a simple, global clinical assessment.

Efficacy is assessed in this study according to patient's reporting outcomes including the assessment of skin pain, pruritus, but also drainage and odour, Dermatology Life Quality Index, HIS-QoL.

The description of specific scores, index, or PROs used as efficacy outcome have been previously described in the definitions and in the description of the Phase IIa trial in Example 1, specifically the following: the International Hidradenitis Suppurativa Severity Score System (IHS4), the Hidradenitis Suppurativa Clinical Response (HiSCR50), the Hidradenitis Suppurativa Area and Severity Index (HASI), the Hidradenitis Suppurativa-Physician Global Assessment (HS-PGA), the Pain Numerical Rating Scale (NRS Pain), the Dermatology Life Quality Index (DLQI), the Hidradenitis Suppurativa Quality of Life (HiS-QoL), the Functional Assessment of Chronic Illness Therapy (FACIT-Fatigue), the Patient Global Impression of Change (PGI-C), and the Patient Global Impression of Severity (PGI-S).

Additional instruments include the Pruritus Numerical Rating Scale (NRS Pruritus), the Hospital Anxiety and Depression Scale (HADS), and the Hidradenitis Suppurativa odour and drainage scale (HODS), Hospital Anxiety and Depression Scale (HADS) described below.

Pruritus Numerical Rating Scale (NRS Pruritus): The HS Pruritus NRS is an endpoint for the assessment of HS-related pruritus severity for clinical trials with patients with HS. The Patient Global Assessment of HS Pruritus assess the worst HS pruritus. Ratings range from 0 (no itch) to 10 (HS worst imaginable itch). It is a unidimensional measure of pruritus intensity and can be administered daily with minimal trial participant burden. Recall period is 24 h and response is given by an 11-point scale ranging from 0 (“no itch”) to 10 (“worst imaginable itch”). Trial participants are asked to rate the intensity of their itch using this scale.

Hidradenitis Suppurativa odour and drainage scale (HODS): The HODS is an 8-item scale developed to assess the HS-related drainage and odour in patients. It covers two domains: drainage (5 items) and odour (3 items). The response options range from 1-5. Higher scores depict worse outcome of the concept being assessed.

Hospital Anxiety and Depression Scale (HADS): The HADS is an instrument for screening anxiety and depression in non-psychiatric populations. The HADS consists of 14 items, 7 each for anxiety and depression symptoms; possible scores range from 0 to 21 for each subscale.

Additional instruments included in part 2 (phase III) only include the Work Productivity and Activity Impairment Questionnaire for HS (WPAI-HS) and EuroQol 5 dimensions 5-level (EQ-5D-5L) described below.

Work Productivity and Activity Impairment Questionnaire for HS (WPAI-HS): The WPAI-HS is 6-item instrument to assess the effect of hidradenitis suppurativa on ability to work and perform normal daily activities.

EuroQol 5 dimensions 5-level (EQ-5D-5L): Assessment of changes in quality of life using the EuroQol-5D-5L. The descriptive system comprises five dimensions: mobility, self-care, usual activities, pain/discomfort, and anxiety/depression. Each dimension has 5 levels: no problems, slight problems, moderate problems, severe problems, and extreme problems. The EQ VAS records the patient's self-rated health on a vertical visual analogue scale, where the endpoints are labelled ‘The best health you can imagine’ and ‘The worst health you can imagine’.

### Statistical Methods:

### Part I (Phase IIb)

The primary analysis of primary endpoint consists of a combination of MCPMod-based testing with respect to a non-flat dose response curve and an assessment of quantitative treatment benefit.

To account for the repeated nature of the data and the covariates in the model, MMRM analysis will be carried out as the basis of MCPMod analysis.

Percent change from baseline in dT count will be used as the response variable in the MMRM. The MMRM will include fixed categorical effects of treatment at each visit, TNFi status at baseline, and categorical baseline dT count at each visit. Visit will be treated as the repeated measure with an unstructured covariance structure used to model the within-patient measurements.

### Part 2 (Phase III)

The primary endpoint is percent change from baseline in dT count at Week 16. Restricted maximum likelihood estimation based on a mixed-effect model for repeated measures analysis will be used to obtain adjusted means for the treatment effects. This model will include categorical fixed effects for treatment at each visit, TNFi status at baseline, and categorical baseline dT count at each visit. The primary treatment comparisons will be the contrast between treatment at Week 16.

Significance tests will be based on adjusted treatment differences using a one-sided α of 0.025.

### Safety:

Safety will be assessed descriptively based on the following observations and examinations as previously described in Example I: Physical examination, Vital signs, Clinical laboratory values (haematology, clinical chemistry, coagulation, infection testing and urinalysis), 12-lead ECG, AEs, Serious AEs (SAEs), Intensity of AEs assessed by CTCAE grading version 5.0, and Suicidality. Physical examinations are performed at the time points specified in the Flowchart (FIGS. 11A & 11B).

Part I and II: Occurrence of treatment emergent adverse events (TEAEs)

### Further Objectives:

Further objectives evaluate further measures of efficacy including effect size up to Week 16, the pharmacokinetics (PK), the immunogenicity (anti-drug antibody [ADA] and neutralizing antibody [NAb]) of spesolimab, and explores biomarkers related to changes in HS and IL36 pathway after treatment (assessment of gene expression levels in skin biopsies and whole blood, evaluation of serum proteomics and imaging markers acquired by ultrasound [US])

### Discussion

The primary objectives of the dose finding phase were to demonstrate a non-flat dose response curve, evaluate the quantitative treatment effect size, and evaluate the dose-response relationship based on the primary endpoint of percent change from baseline in dT count at Week 8. Additionally, the dose finding phase was used to determine an optimal dose for Part 2 by incorporating information of available safety and of efficacy based on the primary endpoint of percent change from baseline in dT count at Week 8. Efficacy of spesolimab is evaluated on the basis of percent change from baseline in dT count at Week 8 and week 16; absolute change from baseline in IHS4 value at Week 8; and absolute change from baseline in IHS4 value at Week 16.

Key secondary endpoints may be used to demonstrate efficacy including: absolute change from baseline in IHS4 value at Week 16; absolute change from baseline in Hidradenitis Suppurativa Area Severity Index (HASI) score at Week 16; achievement of Hidradenitis Suppurativa Clinical Response (HiSCR50) at Week 16; percent change from baseline in abscess count at Week 16; achievement of at least a 50% reduction in dT count at Week 16 relative to baseline; achievement of at least a 50% reduction in abscess and dT (AdT) count at Week 16 relative to baseline; achievement of at least a 50% reduction in ANdT count at Week 16 relative to baseline; and achievement of at least 30% reduction from baseline in Numerical rating Scale (NRS30) in Patient's Global Assessment of HS pain at Week 16.

Secondary objectives were to demonstrate non-flat dose response curve based on selected secondary and further efficacy endpoints at Week; to evaluate the quantitative treatment effect size of percent change from baseline in dT count at Week 16 and absolute change from baseline in International Hidradenitis Suppurativa Severity Score System (IHS4) value at Week 16; to evaluate the sustainability of spesolimab efficacy up to Week 24 and up to Week 50; and to assess safety of spesolimab.

The primary objective of the confirmatory phase was to demonstrate superiority of one dosing regimen (loading and maintenance dose) of spesolimab (identified on the basis of Phase IIb data analysis) against placebo in the mean percent change from baseline in dT count at Week 16.

While certain aspects and embodiments of the invention have been described, these have been presented by way of example only, and are not intended to limit the scope of the invention. Indeed, the novel methods and systems described herein may be embodied in a variety of other forms without departing from the spirit thereof. The accompanying claims and their equivalents are intended to cover such forms or modifications as would fall within the scope and spirit of the invention.

All patents and/or publications including journal articles cited in this disclosure are expressly incorporated herein by reference.

1. A method of treating, preventing or ameliorating hidradenitis suppurativa (HS) in a patient, said method comprising administering or having administered to the patient a therapeutically effective amount of an anti-IL-36R antibody, wherein the anti-IL-36R antibody is administered in a loading dose of at least 1800 mg comprising one or more parenteral doses of 450 mg, 600 mg, 900 mg, 1200 mg, 1800 mg, 2000 mg, 2400 mg, or 3000 mg of said anti-IL-36R antibody.

2. A method of treating moderate to severe HS in a patient, comprising administering or having administered to the patient a therapeutically effective amount of an anti-IL-36R antibody, wherein the anti-IL-36R antibody is administered in a loading dose of at least 1800 mg comprising one or more parenteral doses of 450 mg, 600 mg, 900 mg, 1200 mg, 1800 mg, 2000 mg, 2400 mg, or 3000 mg of said anti-IL-36R antibody.

3. A method of reducing or alleviating signs and symptoms of HS in a patient, said method comprising administering or having administered to the patient a therapeutically effective amount of an anti-IL-36R antibody, wherein the anti-IL-36R antibody is administered in a loading dose of at least 1800 mg comprising one or more parenteral doses of 450 mg, 600 mg, 900 mg, 1200 mg, 1800 mg, 2000 mg, 2400 mg, or 3000 mg of said anti-IL-36R antibody.

4. A method of reducing the severity and duration of HS, said method comprising administering or having administered to the patient a therapeutically effective amount of an anti-IL-36R antibody, wherein the anti-IL-36R antibody is administered in a loading dose of at least 1800 mg comprising one or more parenteral doses of 450 mg, 600 mg, 900 mg, 1200 mg, 1800 mg, 2000 mg, 2400 mg, or 3000 mg of said anti-IL-36R antibody.

5. A method of treating a skin disorder associated with HS, said method comprising administering or having administered to the patient a therapeutically effective amount of an anti-IL-36R antibody, wherein the anti-IL-36R antibody is administered in a loading dose of at least 1800 mg comprising one or more parenteral doses of 450 mg, 600 mg, 900 mg, 1200 mg, 1800 mg, 2000 mg, 2400 mg, or 3000 mg of said anti-IL-36R antibody.

6. The method according to claim 1, wherein the anti-IL-36R antibody comprises: a) a light chain variable region comprising the amino acid sequence of SEQ ID NO: 26 (L-CDR1); the amino acid sequence of SEQ ID NO: 35, 102, 103, 104, 105 106 or 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

7. The method according to claim 1, wherein the anti-IL-36R antibody comprises:
a) a light chain variable region comprising the amino acid sequence of SEQ ID NO: 26 (L-CDR1); the amino acid sequence of SEQ ID NO: 102 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
b) a light chain variable region comprising the amino acid sequence of SEQ ID NO: 26 (L-CDR1); the amino acid sequence of SEQ ID NO: 103 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
c) light chain variable region comprising the amino acid sequence of SEQ ID NO: 26 (L-CDR1); the amino acid sequence of SEQ ID NO: 104 (L-CDR2);
the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
d) a light chain variable region comprising the amino acid sequence of SEQ ID NO: 26 (L-CDR1); the amino acid sequence of SEQ ID NO: 105 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
e) a light chain variable region comprising the amino acid sequence of SEQ ID NO: 26 (L-CDR1); the amino acid sequence of SEQ ID NO: 106 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3);
f) a light chain variable region comprising the amino acid sequence of SEQ ID NO: 26 (L-CDR1); the amino acid sequence of SEQ ID NO: 140 (L-CDR2); the amino acid sequence of SEQ ID NO: 44 (L-CDR3); and b) a heavy chain variable region comprising the amino acid sequence of SEQ ID NO: 53 (H-CDR1); the amino acid sequence of SEQ ID NO: 62, 108, 109, 110 or 111 (H-CDR2); the amino acid sequence of SEQ ID NO: 72 (H-CDR3).

8. The method according to claim 1, wherein the anti-IL-36R antibody comprises:
a) a light chain variable region comprising the amino acid sequence of SEQ ID NO: 77; and a heavy chain variable region comprising the amino acid sequence of SEQ ID NO: 87; or
b) a light chain variable region comprising the amino acid sequence of SEQ ID NO: 77; and a heavy chain variable region comprising the amino acid sequence of SEQ ID NO: 88; or
c) a light chain variable region comprising the amino acid sequence of SEQ ID NO: 77; and a heavy chain variable region comprising the amino acid sequence of SEQ ID NO: 89; or
d) a light chain variable region comprising the amino acid sequence of SEQ ID NO: 80; and a heavy chain variable region comprising the amino acid sequence of SEQ ID NO: 87; or
e) a light chain variable region comprising the amino acid sequence of SEQ ID NO: 80; and a heavy chain variable region comprising the amino acid sequence of SEQ ID NO: 88; or
f) a light chain variable region comprising the amino acid sequence of SEQ ID NO: 80; and a heavy chain variable region comprising the amino acid sequence of SEQ ID NO: 89; or
g) a light chain variable region comprising the amino acid sequence of SEQ ID NO: 85; and a heavy chain variable region comprising the amino acid sequence of SEQ ID NO: 100; or
h) a light chain variable region comprising the amino acid sequence of SEQ ID NO: 85; and a heavy chain variable region comprising the amino acid sequence of SEQ ID NO:101; or
i) a light chain variable region comprising the amino acid sequence of SEQ ID NO: 86; and a heavy chain variable region comprising the amino acid sequence of SEQ ID NO: 100; or
j) a light chain variable region comprising the amino acid sequence of SEQ ID NO: 86; and a heavy chain variable region comprising the amino acid sequence of SEQ ID NO:101.

9. The method according to claim 1, wherein the anti-IL-36R antibody comprises:
a) a light chain comprising the amino acid sequence of SEQ ID NO: 115; and a heavy chain comprising the amino acid sequence of SEQ ID NO: 125; or
b) a light chain comprising the amino acid sequence of SEQ ID NO: 115; and a heavy chain comprising the amino acid sequence of SEQ ID NO: 126; or
c) a light chain comprising the amino acid sequence of SEQ ID NO: 115; and a heavy chain comprising the amino acid sequence of SEQ ID NO: 127; or
d) a light chain comprising the amino acid sequence of SEQ ID NO: 118; and a heavy chain comprising the amino acid sequence of SEQ ID NO: 125; or
e) a light chain comprising the amino acid sequence of SEQ ID NO: 118; and a heavy chain comprising the amino acid sequence of SEQ ID NO: 126; or
f) a light chain comprising the amino acid sequence of SEQ ID NO: 118; and a heavy chain comprising the amino acid sequence of SEQ ID NO: 127; or
g) a light chain comprising the amino acid sequence of SEQ ID NO: 123; and a heavy chain comprising the amino acid sequence of SEQ ID NO: 138; or
h) a light chain comprising the amino acid sequence of SEQ ID NO: 123; and a heavy chain comprising the amino acid sequence of SEQ ID NO: 139; or
i) a light chain comprising the amino acid sequence of SEQ ID NO: 124; and a heavy chain comprising the amino acid sequence of SEQ ID NO: 138.

10. The method of claim 1, wherein the anti-IL36-R antibody, or antigen binding portion thereof, is spesolimab.

11. The method of claim 1, wherein the anti-IL-36R antibody is administered in four intravenous or subcutaneous doses of 450 mg.

12. The method of claim 1, wherein the anti-IL-36R antibody is administered in three, or four intravenous or subcutaneous doses of 600 mg.

13. The method of claim 1, wherein the anti-IL-36R antibody is administered in two, three, or four intravenous or subcutaneous doses of 900 mg.

14. The method of claim 1, wherein the anti-IL-36R antibody is administered in two, three, or four intravenous or subcutaneous doses of 1200 mg.

15. The method of claim 1, wherein the anti-IL-36R antibody is administered in two, three, or four intravenous or subcutaneous doses of 1800 mg.

16. The method of claim 1, wherein the anti-IL-36R antibody is administered in two, three, or four intravenous or subcutaneous doses of 2000 mg.

17. The method of claim 1, wherein the anti-IL-36R antibody is administered in one or two intravenous or subcutaneous doses of 2400 mg.

18. The method of claim 1, wherein the anti-IL-36R antibody is administered in one or two intravenous or subcutaneous doses of 3000 mg.

19. The method of claim 1, wherein 2, 3, or 4 intravenous or subcutaneous loading doses are administered at qw, q2w, q4w intervals.

20. The method of claim 1, further comprising subcutaneously administering a maintenance dose of at least 1200 mg to the subject comprising at least one dose of 300 mg, 450 mg, 600 mg, 900 mg, 1200 mg, 1800 mg, 2400 mg, or 3000 mg of said anti-IL-36R antibody following the last loading dose.

21. The method of claim 20, wherein the maintenance dose of the anti-IL-36R antibody is administered in at least four subcutaneous doses of 300 mg; three-four subcutaneous doses of 450 mg; two-four subcutaneous doses of 600 mg; two-four subcutaneous doses of 900 mg; one-four subcutaneous doses of 1200 mg; one-four subcutaneous doses of 1800 mg; one-two subcutaneous doses of 2400 mg; or one-two subcutaneous doses of 3000 mg.

22. The method of claim 20, wherein said maintenance dose of the anti-IL-36R antibody is delivered subcutaneously qw, q2w, or q4w at weeks 4, 5, 6, and/or 7 following the last intravenous dose.

23. The method of claim 20, further comprising a maintenance therapy comprising subcutaneously administering to the subject 600 mg, 900 mg, 1200 mg, 1800 mg, 2400 mg, or 3000 mg of said anti-IL-36R antibody biweekly following the last dose of the maintenance dosing period and/or beginning after week 8.

24. The method according to claim 1, wherein the administration results in one or more of the following efficacy endpoints:
a) Percent change from baseline in dT count at Weeks 8 and/or 16;
b) Absolute change from baseline in IHS4 value at Week 8 and/or 16;
c) Absolute change from baseline in Hidradenitis Suppurativa Area Severity Index (HASI) score at Week 16;
d) Achievement of Hidradenitis Suppurativa Clinical Response (HiSCR50) at Week 16;
e) Percent change from baseline in abscess count at Week 16;
f) Achievement of at least a 50% reduction in dT count at Week 16 relative to baseline;
g) Achievement of at least a 50% reduction in abscess and dT (AdT) count at Week 16 relative to baseline;
h) Achievement of at least a 50% reduction in ANdT count at Week 16 relative to baseline;
i) Achievement of at least 30% reduction from baseline in Numerical rating Scale (NRS30) in Patient's Global Assessment of HS pain at Week 16;
j) Percent change from baseline in dT count at each scheduled assessment;
k) Achievement of at least 50% reduction from baseline in dT count at each scheduled assessment;
l) Occurrence of complete elimination of dT at each scheduled assessment;
m) Time to first occurrence of complete elimination of dT during the treatment period;
n) Percent change from baseline in abscess count at each scheduled assessment;
o) Achievement of at least 50% reduction from baseline in abscess count at each scheduled assessment;
p) Occurrence of complete elimination of abscess at each scheduled assessment;
q) Percent change from baseline in total AdT count at each scheduled assessment;
r) Achievement of at least 50% reduction from baseline in AdT count at each scheduled assessment;
s) Percent change from baseline in inflammatory nodule (N) count at each scheduled assessment;
t) Achievement of at least 30% reduction from baseline in Numerical rating scale (NRS30) in Patient's Global Assessment of HS pain at each scheduled assessment;
u) Absolute change from baseline in Hidradenitis Suppurativa Quality of Life (HIS-QoL) total score at each scheduled assessment;
v) Absolute change from baseline in FACIT-Fatigue scale score at each scheduled assessment;
w) Absolute change from baseline in Dermatology Life Quality Index (DLQI) score at each scheduled assessment;
x) Absolute change from baseline in Patient Global Impression of Change (PGI-C) score over time;
y) Absolute change from baseline in Patient Global Impression of Severity (PGI-S) score over time;
z) Change from baseline in Hidradenitis Suppurativa odour and drainage scale (HODS) at each scheduled assessment;
aa) Change from baseline in NRS Pruritus at each scheduled assessment;
bb) Change from baseline in Hospital Anxiety and Depression Scale (HADS) at each scheduled assessment;
cc) Percent change from baseline in total ANdT count at each scheduled assessment; or
dd) Achievement of at least 50% reduction from baseline in ANdT count at each scheduled assessment.

25. A method of preventing the recurrence of HS in a patient treated with one or more parenteral, dose(s) of the anti-IL-36R antibody according to claim 1, said method comprising administering to the patient a prophylactically effective amount of the anti-IL-36R antibody in one or more intravenous or subcutaneous doses.

26. A method of achieving a Hidradenitis Suppurativa Clinical Response (HiSCR50) score in a patient treated with one or more parenteral dose(s) of the anti-IL-36R antibody according to claim 1, said method comprising administering to the patient an effective amount of the anti-IL-36R antibody in one or more intravenous or subcutaneous doses.

27. A method of achieving a complete resolution of HS symptoms in a patient treated with one or more parenteral dose(s) of the anti-IL-36R antibody according to claim 1, said method comprising administering to the patient an effective amount of the anti-IL-36R antibody in one or more intravenous or subcutaneous doses wherein the HS symptoms comprise inflammatory lesions, abscesses, draining fistulae/sinus tracts or tunnels (dT), HS associated inflammation (erythema, induration, open ulcers), HS associated infection, and/or HS associated pain.

28. The method according to claim 25, wherein at least 10%, 20%, 30%, 40%, 50%, 60%, 70% or 80% of the patients achieve clinical responsiveness as measured by Hidradenitis Suppurativa Clinical Response score (HiSCR) defined as at least a 50% reduction from baseline in the total AN count, with no increase in the abscess or draining fistula count at Week 8, 12, 16, 20, 24, 36, 48, 52, 60 or 72 of the treatment.

29. The method according to claim 25, wherein at least 10%, 20%, 30%, 40%, 50%, 60%, 70% or 80% of the patients show significant clinical improvement as measured by percent change from baseline in total AN count at Week 8, 12, 16, 20, 24, 36, 48, 52, 60 or 72 of the treatment.

30. The method according to claim 25, wherein at least 10%, 20%, 30%, 40%, 50%, 60%, 70% or 80% of the patients show significant clinical improvement in the occurrence of HS flare (the incidence of flare defined as at least 25% increase in AN count with a minimum increase of 2 relative to baseline total AN count at Week 8, 12, 16, 20, 24, 36, 48, 52, 60 or 72 of the treatment.

31. The method according to claim 25, wherein at least 10%, 20%, 30%, 40%, 50%, 60%, 70% or 80% of the patients show significant clinical improvement as measured by a change in percent change from baseline in total dT count at Week 8, 12, 16, 20, 24, 36, 48, 52, 60 or 72 of the treatment.

32. The method according to claim 25, wherein at least 10%, 20%, 30%, 40%, 50%, 60%, 70% or 80% of the patients show complete elimination of dT at Week 8, 12, 16, 20, 24, 36, 48, 52, 60 or 72 of the treatment.

33. The method according to claim 25, wherein at least 10%, 20%, 30%, 40%, 50%, 60%, 70% or 80% of the patients show significant clinical improvement as measured by an absolute change from baseline in International Hidradenitis Suppurativa Severity Score system (IHS4) at Week 8, 12, 16, 20, 24, 36, 48, 52, 60 or 72 of the treatment.

34. The method according to claim 25, wherein at least 10%, 20%, 30%, 40%, 50%, 60%, 70% or 80% of the patients show significant clinical improvement as measured by an absolute change from baseline in Hidradenitis Suppurativa Area and Severity Index (HASI) at Week 8, 12, 16, 20, 24, 36, 48, 52, 60 or 72 of the treatment.

35. The method according to claim 25, wherein at least 10%, 20%, 30%, 40%, 50%, 60%, 70% or 80% of the patients show 30% reduction from baseline in Numerical rating scale (NRS30) in Patient's Global Assessment of HS Pain at Week 8, 12, 16, 20, 24, 36, 48, 52, 60 or 72 of the treatment.

36. The method according to claim 25, wherein at least 10%, 20%, 30%, 40%, 50%, 60%, 70% or 80% of the patients show a score of 0 or 1 measured by the Physician Global Assessment (PGA) at Week 8, 12, 16, 20, 24, 36, 48, 52, 60 or 72 of the treatment.

37. The method according to claim 25, wherein at least 10%, 20%, 30%, 40%, 50%, 60%, 70% or 80% of the patients show clinical improvement as measured by absolute change from baseline in the Dermatology life quality index (DLQI) Score at Week 8, 12, 16, 20, 24, 36, 48, 52, 60 or 72 of the treatment.

38. The method according to claim 25, wherein at least 10%, 20%, 30%, 40%, 50%, 60%, 70% or 80% of the patients show clinical improvement as measured by absolute change from baseline in the Hidradenitis suppurative quality of life (HIS-QOL) Total Score at Week 8, 12, 16, 20, 24, 36, 48, 52, 60 or 72 of the treatment.

39. A method of treating HS in a patient, comprising administering to the patient a therapeutically effective amount of one or more intravenous dose(s) or subcutaneous dose(s) of the anti-IL-36R antibody according to claim 1, followed by one or more subcutaneous dose(s) of the anti-IL-36R antibody.

40. The method of claim 39, wherein one, two, three, or four intravenous dose(s) of the anti-IL-36R antibody is/are followed by one, two, three, or subcutaneous dose(s) of the anti-IL-36R antibody.

41. The method according to claim 39, wherein each of the one or more intravenous dose(s) or subcutaneous dose(s) comprises 450 mg, 600 mg, 900 mg, 1200 mg, 1800 mg, 2000 mg, 2400 mg, or 3000 mg of the anti-IL-36R antibody and each of the one or more subcutaneous dose(s) comprises 300 mg, 450 mg, 600 mg, 900 mg, 1200 mg, 1800 mg, 2400 mg, or 3000 mg of the anti-IL-36R antibody.

42. A method of treating HS in a patient, comprising:
a. obtaining a biological sample from said patient, wherein the biological sample is obtained from source comprising lesional skin or whole blood;
b. determining the gene express profile of one or more of genes; and
(c) administering to the patient an effective amount of the anti-IL-36R antibody according to claim 1.

43. The method of claim 42, wherein the one or more of genes are IL12B, IL1β, IL6, CXCL1, IL23A, TNF, IL17C, IL24 or IL1B in lesional skin, and IL1β, S100A9, S100A12, S100A8, MMP25, MMP9 or CD177 in whole blood.


SEQUENCE LISTING (140 sequences)

SEQ ID NO: 1 - protein, 108 aa, Mus sp.
QIVLTQSPAIMSASLGERVTMTCTASSSVSSSYLHWYQKKPGSSPKLWVYSTSNLASGVPVRFSGSGSGTSYSLTISSMEAEDAATYYCHQHHRSPVTFGSGTKLEMK

SEQ ID NO: 2 - protein, 107 aa, Mus sp.
DIQMTQSPASQSASLGESVTFTCLASQTIGTWLAWYQQRPGKSPQLLIYAATSLADGVPSRFSGSGSGTQFSFNIRSLQAEDFASYYCQQVYTTPLTFGGGTKLEIK

SEQ ID NO: 3 - protein, 107 aa, Mus sp.
DIQMTQSPASQSASLGESVTFTCLASQTIGTWLGWYQQKPGKSPQLLIYRSTTLADGVPSRFSGSGSGTKFSFKISSLQAADFASYYCQQLYSAPYTFGGGTKLEIR

SEQ ID NO: 4 - protein, 112 aa, Mus sp.
DVLLTQTPLSLPVSLGDQASISCRSSQNIVHSNGNTYLQWYLQKPGQSPKLLIYKVSNRFSGVPDRFSGSGSGTDFTLKISRVEAEDLGVYYCFQGSHVPFTFGAGTKLELK

SEQ ID NO: 5 - protein, 107 aa, synthetic construct
DIQMTQTTSSLSASLGDRVTISCRASQDIYKYLNWYQQKPDGTLKLLIYYTSGLHSGVPSRFSGSGSGTDFSLTISNLEPEDIATYFCQQDSKFPWTFGGDTKLEIK

SEQ ID NO: 6 - protein, 108 aa, Mus sp.
QIVLTQSPAIMSASLGERVTMTCTASSSVSSSYFHWYQQKPGSSPKLWIYRTSNLASGVPGRFSGSGSGTSYSLTISSMEAEDAATYYCHQFHRSPLTFGAGTKLELK

SEQ ID NO: 7 - protein, 107 aa, Mus sp.
DIVMTQSQKFLSTSVGVRVSVTCKASQDVGTNVLWYQQKIGQSPKPLIYSASYRHSGVPDRFTGSGSGTDFTLIISNVQSEDLAEYFCQQYSRYPLTFGPGTKLELK

SEQ ID NO: 8 - protein, 107 aa, Mus sp.
DIVMTQSQKFLSTSVGVRVSVTCKASQDVGTNVLWYQQKIGQSPKALIYSASYRHSGVPDRFTGSGSGTDFTLIITNVQSEDLAEYFCQQYSRYPLTFGPGTKLELK

SEQ ID NO: 9 - protein, 107 aa, Mus sp.
DIVMTQSQKFMSATVGGRVNITCKASQNVGRAVAWYQQKPGQSPKLLTHSASNRYTGVPDRFTGSGSGTDFTLTITNMQSEDLADYFCQQYSSYPLTFGAGTKLDLK

SEQ ID NO: 10 - protein, 107 aa, Mus sp.
DIQMTQSPASQSASLGESVTFSCLASQTIGTWLGWYQQKPGKSPQLLIYRATSLADGVPSRFSGSGSGTNFSFKISSLQAEDLASYYCQQLYSGPYTFGGGTKLEIR

SEQ ID NO: 11 - protein, 119 aa, Mus sp.
QVQLQQSGTELLKPGASVKLSCKASGNTVTSYWMHWVKQRPGQGLEWIGEILPSTGRTNYNENFKGKAMLTVDKSSSTAYMQLSSLASEDSAVYYCTIVYFGNPWFAYWGQGTLVTVSA

SEQ ID NO: 12 - protein, 125 aa, Mus sp.
EVQLQQSGPELVKPGASVKLSCKASGYTFTDNYMNWVRQSHGKSLEWIGRVNPSNGDTKYNQNFKGKATLTVDKSLSTAYMQLNGLTSEDSAVYYCGRTKNFYSSYSYDDAMDYWGQGTSVTVSS

SEQ ID NO: 13 - protein, 124 aa, Mus sp.
EVQLQQSGAEFVRPGASVKFSCTASGFNIKDDYIHWVRQRPEQGLEWVGRIDPANGNTKYAPKFQDKATITADTSSNTAYLQLSSLTSEDTAVYYCAKSFPNNYYSYDDAFAYWGQGTLVTVSA

SEQ ID NO: 14 - protein, 118 aa, Mus sp.
QVQLKESGPVLVAPSQSLSITCTVSGFSLTKFGVHWIRQTPGKGLEWLGVIWAGGPTNYNSALMSRLTISKDISQSQVFLRIDSLQTDDTAMYYCAKQIYYSTLVDYWGQGTSVTVSS

SEQ ID NO: 15 - protein, 120 aa, Mus sp.
QVQLKESGPGLVAPSQSLFITCTVSGFSLSSYEINWVRQVPGKGLEWLGVIWTGITTNYNSALISRLSISKDNSKSLVFLKMNSLQTDDTAIYYCARGTGTGFYYAMDYWGQGTSVTVSS

SEQ ID NO: 16 - protein, 119 aa, Mus sp.
QVQLQQPGADFVRPGASMRLSCKASGYSFTSSWIHWVKQRPGQGLEWIGEINPGNVRTNYNENFRNKATLTVDKSSTTAYMQLRSLTSADSAVYYCTVVFYGEPYFPYWGQGTLVTVSA

SEQ ID NO: 17 - protein, 119 aa, Mus sp.
QVQLKESGPGLVAPSQSLSITCTVSGFSLTNYAVHWVRQFPGKGLEWLGVIWSDGSTDFNAPFKSRLSINKDNSKSQVFFKMNSLQIDDTAIYYCARKGGYSGSWFAYWGQGTLVTVSA

SEQ ID NO: 18 - protein, 119 aa, Mus sp.
QVQLKESGPGLVAPSQSLSITCTVSGFSLTNYAVHWVRQFPGKGLEWLGVIWSDGSTDYNAPFKSRLSINKDNSKSQVFFKMNSLQTDDTAIYYCARKGGYSGSWFAYWGQGTLVTVSA

SEQ ID NO: 19 - protein, 117 aa, Mus sp.
QVQLKESGPVLVAPSQSLSITCTVSGFSLTNYGVHWVRQPPGKGLEWLGVIWPVGSTNYNSALMSRLSIHKDNSKSQVFLRMNSLQTDDTAIYYCAKMDWDDFFDYWGQGTTLTVSS

SEQ ID NO: 20 - protein, 124 aa, Mus sp.
EVQLQQSGAELVRPGASVRLSCTASGFNIKDDYIHWVRQRPKQGLEWLGRIDPANGNTKYDPRFQDKATITADTSSNTAYLHLSSLTSEDTAVYYCAKSFPDNYYSYDDAFAYWGQGTLVTVSA

SEQ ID NO: 21 - protein, 12 aa, Mus sp.
TASSSVSSSYLH

SEQ ID NO: 22 - protein, 11 aa, Mus sp.
LASQTIGTWLA

SEQ ID NO: 23 - protein, 11 aa, Mus sp.
LASQTIGTWLG

SEQ ID NO: 24 - protein, 16 aa, Mus sp.
RSSQNIVHSNGNTYLQ

SEQ ID NO: 25 - protein, 11 aa, Mus sp.
RASQDIYKYLN

SEQ ID NO: 26 - protein, 12 aa, Mus sp.
TASSSVSSSYFH

SEQ ID NO: 27 - protein, 11 aa, Mus sp.
KASQDVGTNVL

SEQ ID NO: 28 - protein, 11 aa, Mus sp.
KASQNVGRAVA

SEQ ID NO: 29 - protein, 11 aa, Mus sp.
LASQTIGTWLG

SEQ ID NO: 30 - protein, 7 aa, Mus sp.
STSNLAS

SEQ ID NO: 31 - protein, 7 aa, Mus sp.
AATSLAD

SEQ ID NO: 32 - protein, 7 aa, Mus sp.
RSTTLAD

SEQ ID NO: 33 - protein, 7 aa, Mus sp.
KVSNRFS

SEQ ID NO: 34 - protein, 7 aa, Mus sp.
YTSGLHS

SEQ ID NO: 35 - protein, 7 aa, Mus sp.
RTSNLAS

SEQ ID NO: 36 - protein, 7 aa, Mus sp.
SASYRHS

SEQ ID NO: 37 - protein, 7 aa, Mus sp.
SASNRYT

SEQ ID NO: 38 - protein, 7 aa, Mus sp.
RATSLAD

SEQ ID NO: 39 - protein, 9 aa, Mus sp.
HQHHRSPVT

SEQ ID NO: 40 - protein, 9 aa, synthetic construct
QQVYTTPLT

SEQ ID NO: 41 - protein, 9 aa, Mus sp.
QQLYSAPYT

SEQ ID NO: 42 - protein, 9 aa, Mus sp.
FQGSHVPFT

SEQ ID NO: 43 - protein, 9 aa, Mus sp.
QQDSKFPWT

SEQ ID NO: 44 - protein, 9 aa, Mus sp.
HQFHRSPLT

SEQ ID NO: 45 - protein, 9 aa, Mus sp.
QQYSRYPLT

SEQ ID NO: 46 - protein, 9 aa, Mus sp.
QQYSSYPLT

SEQ ID NO: 47 - protein, 9 aa, Mus sp.
QQLYSGPYT

SEQ ID NO: 48 - protein, 10 aa, Mus sp.
GNTVTSYWMH

SEQ ID NO: 49 - protein, 10 aa, Mus sp.
GYTFTDNYMN

SEQ ID NO: 50 - protein, 10 aa, Mus sp.
GFNIKDDYIH

SEQ ID NO: 51 - protein, 10 aa, Mus sp.
GFSLTKFGVH

SEQ ID NO: 52 - protein, 10 aa, Mus sp.
GFSLSSYEIN

SEQ ID NO: 53 - protein, 10 aa, Mus sp.
GYSFTSSWIH

SEQ ID NO: 54 - protein, 10 aa, Mus sp.
GFSLTNYAVH

SEQ ID NO: 55 - protein, 10 aa, Mus sp.
GFSLTNYGVH

SEQ ID NO: 56 - protein, 10 aa, Mus sp.
GFNIKDDYIH

SEQ ID NO: 57 - protein, 17 aa, Mus sp.
EILPSTGRTNYNENFKG

SEQ ID NO: 58 - protein, 17 aa, Mus sp.
RVNPSNGDTKYNQNFKG

SEQ ID NO: 59 - protein, 17 aa, Mus sp.
RIDPANGNTKYAPKFQD

SEQ ID NO: 60 - protein, 16 aa, Mus sp.
VIWAGGPTNYNSALMS

SEQ ID NO: 61 - protein, 16 aa, Mus sp.
VIWTGITTNYNSALIS

SEQ ID NO: 62 - protein, 15 aa, Mus sp.
EINPGNVRTNYNENF

SEQ ID NO: 63 - protein, 16 aa, Mus sp.
VIWSDGSTDFNAPFKS

SEQ ID NO: 64 - protein, 16 aa, Mus sp.
VIWSDGSTDYNAPFKS

SEQ ID NO: 65 - protein, 16 aa, Mus sp.
VIWPVGSTNYNSALMS

SEQ ID NO: 66 - protein, 17 aa, Mus sp.
RIDPANGNTKYDPRFQD

SEQ ID NO: 67 - protein, 10 aa, Mus sp.
VYFGNPWFAY

SEQ ID NO: 68 - protein, 16 aa, Mus sp.
TKNFYSSYSYDDAMDY

SEQ ID NO: 69 - protein, 15 aa, Mus sp.
SFPNNYYSYDDAFAY

SEQ ID NO: 70 - protein, 10 aa, Mus sp.
QIYYSTLVDY

SEQ ID NO: 71 - protein, 12 aa, Mus sp.
GTGTGFYYAMDY

SEQ ID NO: 72 - protein, 10 aa, Mus sp.
VFYGEPYFPY

SEQ ID NO: 73 - protein, 11 aa, Mus sp.
KGGYSGSWFAY

SEQ ID NO: 74 - protein, 9 aa, Mus sp.
MDWDDFFDY

SEQ ID NO: 75 - protein, 15 aa, Mus sp.
SFPDNYYSYDDAFAY

SEQ ID NO: 76 - protein, 108 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIK

SEQ ID NO: 77 - protein, 108 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIK

SEQ ID NO: 78 - protein, 108 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIK

SEQ ID NO: 79 - protein, 108 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIK

SEQ ID NO: 80 - protein, 108 aa, synthetic construct [sequence omitted]

SEQ ID NO: 81 - protein, 108 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIK

SEQ ID NO: 82 - protein, 108 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIK

SEQ ID NO: 83 - protein, 108 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIK

SEQ ID NO: 84 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIK

SEQ ID NO: 85 - protein, 107 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIK

SEQ ID NO: 86 - protein, 107 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIK

SEQ ID NO: 87 - protein, 119 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSS

SEQ ID NO: 88 - protein, 119 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSS

SEQ ID NO: 89 - protein, 119 aa, synthetic construct [sequence omitted]

SEQ ID NO: 90 - protein, 119 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSS

SEQ ID NO: 91 - protein, 119 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSS

SEQ ID NO: 92 - protein, 119 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSS

SEQ ID NO: 93 - protein, 119 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSS

SEQ ID NO: 94 - protein, 119 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSS

SEQ ID NO: 95 - protein, 119 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSS

SEQ ID NO: 96 - protein, 119 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSS

SEQ ID NO: 97 - protein, 119 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSS

SEQ ID NO: 98 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSS

SEQ ID NO: 99 - protein, 119 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSS

SEQ ID NO: 100 - protein, 119 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSS

SEQ ID NO: 101 - protein, 119 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSS

SEQ ID NO: 102 - protein, 7 aa, Mus sp.
RTSTLAS

SEQ ID NO: 103 - protein, 7 aa, Mus sp.
RTSILAS

SEQ ID NO: 104 - protein, 7 aa, Mus sp.
RTSRLAS

SEQ ID NO: 105 - protein, 7 aa, Mus sp.
RTSQLAS

SEQ ID NO: 106 - protein, 7 aa, Mus sp.
RTSKLAS

SEQ ID NO: 107 - protein, 10 aa, Mus sp.
GFSLTDYAVH

SEQ ID NO: 108 - protein, 15 aa, Mus sp.
EILPGVVRTNYNENF

SEQ ID NO: 109 - protein, 15 aa, Mus sp.
EINPGAVRTNYNENF

SEQ ID NO: 110 - protein, 15 aa, Mus sp.
EINPGLVRTNYNENF

SEQ ID NO: 111 - protein, 15 aa, Mus sp.
EINPGSVRTNYNENF

SEQ ID NO: 112 - protein, 330 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 107 aa, synthetic construct [sequence omitted]

SEQ ID NO: 114 - protein, 215 aa, synthetic construct
EIVLTQSPGTLSLSPGERATMSCTASSSVSSSYFHWYQQKPGQAPRLLIYRTSTLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 115 - protein, 215 aa, synthetic construct
EIVLTQSPGTLSLSPGERATMSCTASSSVSSSYFHWYQQKPGQAPRLLIYRTSILASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 116 - protein, 215 aa, synthetic construct
EIVLTQSPGTLSLSPGERATMSCTASSSVSSSYFHWYQQKPGQAPRLWIYRTSRLASGVPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 117 - protein, 215 aa, synthetic construct
EIVLTQSPGTLSLSPGERATMTCTASSSVSSSYFHWYQQKPGQAPRLLIYRTSRLASGVPDRFSGSGSGTDFTLTISRLEPEDFAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 118 - protein, 215 aa, synthetic construct
QIVLTQSPGTLSLSPGERATMTCTASSSVSSSYFHWYQQKPGQAPRLWIYRTSRLASGVPDRFSGSGSGTDFTLTISRLEPEDAATYYCHQFHRSPLTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 119 - protein, 215 aa, synthetic construct
QIVLTQSPGTLSLSPGERVTMSCTASSSVSSSYFHWYQQKPGQAPRLLIYRTSQLASGIPDRFSGSGSGTDFTLTISRLEPEDAATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 120 - protein, 215 aa, synthetic construct
QIVLTQSPGTLSLSPGERATMTCTASSSVSSSYFHWYQQKPGQAPRLLIYRTSKLASGVPDRFSGSGSGTDFTLTISRLEPEDFATYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 121 - protein, 215 aa, synthetic construct
EIVLTQSPGTLSLSPGERATMSCTASSSVSSSYFHWYQQKPGQAPRLLIYRTSHLASGIPGRFSGSGSGTDFTLTISRLEPEDAAVYYCHQFHRSPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 122 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAEYFCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 123 - protein, 214 aa, synthetic construct
EIVMTQSPATLSVSPGVRATLSCKASQDVGTNVLWYQQKPGQAPRPLIYSASYRHSGIPDRFSGSGSGTEFTLTISSLQSEDFAVYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 124 - protein, 214 aa, synthetic construct
EIVMTQSPATLSVSPGVRATLSCKASQDVGTNVLWYQQKPGQAPRPLIYSASYRHSGIPARFSGSGSGTEFTLTISSLQSEDFAEYYCQQYSRYPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 125 - protein, 449 aa, synthetic construct
QVQLVQSGAEVKKPGASVKVSCKASGYSFTSSWIHWVRQAPGQGLEWIGEINPGNVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 449 aa, synthetic construct
QVQLVQSGAEVKKPGASVKVSCKASGYSFTSSWIHWVRQRPGQGLEWIGEINPGNVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 449 aa, synthetic construct
QVQLVQSGAEVKKPGASVKVSCKASGYSFTSSWIHWVKQAPGQGLEWMGEINPGNVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 449 aa, synthetic construct
QVQLVQSGAEVKKPGASVKVSCKASGYSFTSSWIHWVRQRPGQGLEWMGEINPGNVRTNYNENFRNRATLTRDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 449 aa, synthetic construct
QVQLVQSGAEVKKPGASVKVSCKASGYSFTSSWIHWVRQRPGQGLEWMGEILPGVVRTNYNENFRNKVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 130 - protein, 449 aa, synthetic construct
QVQLVQSGAEVKKPGASVKVSCKASGYSFTSSWIHWVRQRPGQGLEWIGEINPGAVRTNYNENFRNRVTMTVDTSISTAYMELSRLRSDDTAVYYCTVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 131 - protein, 449 aa, synthetic construct
QVQLVQSGAEVKKPGASVKVSCKASGYSFTSSWIHWVRQAPGQGLEWIGEINPGLVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 132 - protein, 449 aa, synthetic construct
QVQLVQSGAEVKKPGASVKVSCKASGYSFTSSWIHWVRQAPGQGLEWIGEINPGAVRTNYNENFRNKVTMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 133 - protein, 449 aa, synthetic construct
QVQLVQSGAEVKKPGASVKVSCKASGYSFTSSWIHWVRQAPGQGLEWIGEINPGSVRTNYNENFRNKATMTVDTSISTAYMELSRLRSDDTAVYYCAVVFYGEPYFPYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 449 aa, synthetic construct
QVQLQESGPGLVKPSETLSITCTVSGFSLTDYAVHWIRQPPGKGLEWIGVIWSDGSTDYNAPFKSRVTINKDTSKSQVSFKMSSVQAADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 449 aa, synthetic construct
QVQLQESGPGLVKPSETLSITCTVSGFSLTDYAVHWIRQPPGKGLEWIGVIWSDGSTDYNAPFKSRVTISKDTSKNQVSLKMNSLTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSLKMNSVTVADTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 449 aa, synthetic construct
QVQLQESGPGLVKPSETLSITCTVSGFSLTDYAVHWVRQPPGKGLEWIGVIWSDGSTDYNAPFKSRVTISKDTSKNQVSFKLSSVTVDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 449 aa, synthetic construct
QVQLQESGPGLVAPSETLSLTCTVSGFSLTDYAVHWIRQFPGKGLEWIGVIWSDGSTDFNAPFKSRVTISKDTSKNQVSFKLSSVTTDDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 449 aa, synthetic construct
QVQLQESGPGLVKPSETLSITCTVSGFSLTDYAVHWIRQPPGKGLEWIGVIWSDGSTDYNAPFKSRVTISKDNSKSQVSFKMSSVTADDTAVYYCARKGGYSGSWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 140 - protein, 7 aa, Mus sp.
RTSHLAS